(19) United States
(12) Patent Application Publication
(10) Pub. No.: US 2009/0076294 A1
(43) Pub. Date: Mar. 19, 2009

(54) CONTROLLED NUCLEATION OF SOLUTES IN SOLUTIONS HAVING NET CHARGE TO PROMOTE CRYSTAL GROWTH

(75) Inventors: George R. Agnes, Coquitlam (CA); Michael J. Bogan, Dublin, CA (US); Samuel F.W. Bakhoum, Hanover, NH (US)

Correspondence Address: CHERNOFF, VILHAUER, MCCLUNG & STENZEL, 1600 ODS TOWER, 601 SW SECOND AVENUE, PORTLAND, OR, 97204-3157, US

(73) Assignee: Simon Fraser University, Burnaby (CA)

(21) Appl. No.: 10/588,694

(22) PCT Filed: Feb. 9, 2005
(86) PCT No.: PCT/CA2005/000158

§ 371 Date: Nov. 24, 2008

Related U.S. Application Data
(60) Provisional application No. 60/542,294, filed on Feb. 9, 2004.

(51) Int. Cl. C07C 255/07; C07C 49/78; B01D 9/02

(52) U.S. Cl. 558/426; 568/337; 23/295 R

(57) ABSTRACT

This application is concerned with the controlled nucleation of solutes (i.e. dissolved solids) from solution. It has been found that the energy barrier for dissolved solids to nucleate is affected by the surface charge density of the reaction vessel (and hence the mass-to-charge ratio of vessel). The reaction vessel may, for example, comprise a levitated droplet of the solution having an “excess net charge”. That is, ions present in the vessel of a single polarity are in excess of the counterions of opposite polarity. An increase in the surface charge density of the vessel (and hence a reduction in the mass-to-charge ratio of the vessel) causes the barrier for nucleation to decrease. These findings can be exploited using instruments commonly used in wall-less sample preparation to elicit selective control over the induction of nucleation and subsequent crystallization of target solutes of interest in the condensed phase. The ion induced nucleation phenomenon, in reaction vessels having a desirable surface charge density, is likely to be general for all dissolved solids, ranging from inorganic compounds, to low and high molecular weight organic compounds, including proteins and other molecules. For example the present invention can be used to selectively crystallize a target solute or to separate different solutes from one another based on their propensity to nucleate at different reaction conditions. The different solutes could constitute different compounds or different stereochemical forms of same compound. The invention could also be exploited to controllably select or separate polymorphic forms of a compound (which may often have very different biological activity). The crystals derived from the process could be the subject of further analysis, characterization or manipulation, for example as a prepared sample for MALDI-TOF MS.

### REFERENCE TO RELATED APPLICATIONS

This application claims the benefit of U.S. provisional patent application Ser. No. 60/542,294 filed 9 Feb. 2004 which is hereby incorporated by reference.

### FIELD OF THE INVENTION

This application relates to a method of controllably inducing nucleation of a solute in a solution having a net charge to promote the growth of crystals.

### BACKGROUND OF THE INVENTION

Crystallization of solutes from solution is an important industrial separation and purification process. The first step in the process is the creation of the new phase which is known as nucleation. Crystallization is thus preceded by nucleation, which occurs either spontaneously or is induced by particles or vibration. Experimental studies of the thermodynamics and kinetics of nucleation processes are difficult because of the role of surfaces and impurities in aiding the nucleation process.

The formation of crystals of a solute (i.e. a dissolved solid) is typically described in terms of solute's phase diagram in a given liquid medium. The medium may be defined by its chemical properties (such as concentration of and types of solvents, electrolyte, pH, buffers, impurities, and other solute(s) of interest) and physical properties (such as type of container, temperature, pressure, magnetic fields, electric fields, and gravity). Provided the phase diagram is known, or can be established through experiment, crystals of a dissolved solid can in principle, be obtained. For example, in the case of crystallization of inorganic compounds for which phase transitions and phase diagrams can be measured, theories of crystal nucleation and growth have been applied successfully.[1-7]

However, despite the development of solute nucleation and crystallization theory over a period of at least 100 hundred years, it is often difficult to demonstrate in practice that the formation of a macroscopic crystal of dimensions >1 μm requires the formation of nanoscopic nuclei. It is believed that nuclei on the scale of only a few nanometers form and disappear rapidly in the nucleation medium (i.e. in an equilibrium process). A key step is to coax, through adjustment of the chemical and/or physical description of the nucleation medium, those nuclei to grow to a critical size such that crystallization is induced. The degree of supersaturation of the solute is an important factor, and the solute concentration needed to cause nuclei to form and then grow into crystals is referred to as “critical supersaturation”. Once the nuclei has formed, that nuclei must reach a critical size that is dependent on each solute, such that the solute will spontaneously precipitate onto the nuclei causing it to grow into a crystal rather than shrinking in size and ultimately disappearing.

Theories of crystal growth do not necessarily help the experimentalist determine which conditions in the nucleation medium are necessary for nucleation and growth of crystals for compounds that have not previously been crystallized by experiment. Examples of such compounds include a newly isolated natural product or novel synthesized compounds (such as a new pharmaceutical compound or an analogue of it), or known compounds that have not yet yielded to crystallization attempts (such as soluble or integral-membrane proteins). The crystallization of novel pharmaceutical compounds is in fact regarded by those who synthesize them as an empirical process where proficiency is achieved through experience and trial and error.

These practical issues for solute crystallization have led to a large number of crystallization strategies that have resulted in an even larger number of experimental techniques and methods. Numerous investigators have studied organic compound nucleation and crystal growth in container-less (or wall-less) sample vessels levitated in a medium (typically air at atmospheric pressure). Wall-less sample preparation has been described in detail previously be the inventors [8]. The rationale for the use of container-less vessels in which to effect nucleation and crystal growth was to provide a more homogeneous environment in which a crystal could be grown because there was no liquid:solid interface at the wall of the nucleation vessel or medium (.ie. such as a plastic or glass vial).

One of the first, and most celebrated, experiments based on single particles was the Millikan oil drop experiment, performed in 1909.[9,10] By partially suspending single charged oil droplets in a DC field, Millikan was able to determine the charge of an electron, an experiment for which he was awarded the Nobel prize. Later modification of the experimental apparatus by Wolfgang Paul to include AC fields to trap ions under vacuum led to the development of modem quadrupolar field-based mass spectrometers, and another Nobel prize.[1] The Paul trap technology has since been extended to the trapping of more massive particles by suitable adjustment of the frequencies and amplitudes of the potentials applied to the electrodes of these devices. This resulted in the development of the electrodynamic balance for single aerosol particle characterization. This latter technology has been widely applied to the study of the physical characteristics of single levitated droplets/particles, such as evaporation, charge, and condensation processes.[12-14] Efforts to probe the chemistry occurring in these levitated particles has lead to elegant laser-based probing methods revealing real-time chemical reaction information relevant to atmospheric processes.[15]

An application of the container-less vessel (i.e. a levitated droplet) for nucleation of organic compounds which has received considerable attention is for protein crystallization. Again, the absence of an interface at the solution-container wall was believed to be of significance with respect to avoiding the growth of impure crystals or crystal with defects, and particularly so when the nucleation experiment was performed at low temperature. The types of levitation of containerless nucleation media (otherwise known as levitated droplets) are acoustic, magnetic, electrostatic, optical, and combinations thereof. Most of these types of levitation have been studied with respect to their utility in forming crystals of organic compounds.

A tremendous quantity of research has been performed on gas-phase ion-molecule reactions. In some cases solute nucleation and crystal growth in the presence of a charged entity has been performed. This body of work has clearly shown that ions can act as nuclei for the clustering and growth of aggregates. The structure of these clusters could be of relevance with respect to the nanoscopic nuclei that ultimately grow into crystals, and moreover, gas-phase ion-molecule reactions are widely acknowledged as experiments that provide snapshots of ion solvation in the (liquid) condensed phase. Though in these studies the aggregates are themselves isolated in the gas phase, the aggregate itself could or could not be in a solid or liquid form depending on the temperature of the aggregate.

The scope of ion-molecule reactions is enormous, and those studies span atomic ions and molecular ions as the charge center onto which molecules and atoms cluster around.[1,6] This work has now extended into the gas phase acid-base properties of proteins.[1,7] Synthetic diamond crystal growth is described as the formation of a 13-Carbon center using chemical vapor deposition technology to form C⁻ in the gas phase that then causes nucleation of carbon as diamond, rather than the more thermodynamically stable graphite form.[18, 19]

Prior art studies have shown that it is often difficult to achieve stable nuclei in the gas phase (i.e. without decomposition). In practice it is difficult to achieve a high enough density in the gas phase to form nuclei that can actually give rise to a crystal. While some ion-molecule clustering and nucleation has been observed in the gas phase, analogous experiments which involve ions clustering phenomena in the condensed phase of a medium that has net charge have not been performed. A condensed phase medium that has net charge may mimic the net charge on a gas phase ion. However, condensed phase media in which the mass-to-charge ratio of that medium was adjusted has not been demonstrated or reported in the literature as an experimental variable that influences nucleation in the condensed phase. This is significant because in essentially all condensed phase media in which nucleation has been studied, regardless of the abundance of charged species present in such media, those media had an overall net electrical charge of zero (i.e. the nucleation media were neutral).

The possibility that the net charge (and thus the mass-to-charge ratio) of the nucleation medium could influence condensed phase nucleation or crystal growth does not appear to have been previously disclosed or demonstrated in the prior art. Tang et al. have described an investigation of solute nucleation in levitated solution droplets[20] This manuscript dealt with the determination of critical supersaturation for the nucleation of NaCl and NH₄SO₄ systems, which is a process believed to be important in tropospheric aerosols. However, there is no mention of variation of the net charge of the nucleation medium, nor the mass-to-charge ratio of the nucleation medium, which was a levitated droplet. As an another example of the literature from the atmospheric community regarding the transition from liquid phase to solid phase in aerosols, the Leisner group has published several reports on the nucleation of atmospherically relevant compounds in levitated droplets.[21,22] Still others use the electrodynamic balance to study reactions at the particle-air interface (ie. heterogeneous reactions),[23-26] and phase transfer and freezing processes.[27, 21, 28, 29] The motivation for conducting many of these studies was the hypothesis that reactions at the droplet/particle-air interface are of primary relevance with respect to understanding the science of the troposphere and stratosphere.

Other reports of chemistry in levitated droplets range from simple acid-base reaction in a picoliter vessel,[30] to reactions that were photochemically initiated.[31-35] Optical levitation has also been used to study similar reactions. Cederfelt and co-workers described the charge limit for a droplet in which NaCl could be crystallized in a levitated droplet.[36] Basically, they were interested in determining the maximum net charge that could be contained in a droplet which dried to a solid residue of NaCl without the droplet undergoing a Coulomb explosion. Coulomb explosion is a process by which a droplet with net charge fragments because the repulsive force of its net charge exceeds the attractive force of the droplet, which is a function of the droplet's surface tension and radius.[37-39] There was no mention in the Cederfelt and co-worker manuscript of a change in the nucleation rate or abundance of nucleation sites as a function of net charge (likely because they were nucleating NaCl which is arguably the easiest compound to form crystals of).

In related studies that are of direct relevance to Coulomb explosion, the charge loss from single levitated droplets has been described in a number of manuscripts in recent years.[38-45] The process of a droplet with net charge releasing some of that charge (i.e. Coulomb explosion) has received considerable attention because of the introduction of Electrospray Mass Spectrometry for the characterization of biomacromolecules by John Fenn.[46-48] Interestingly, a charged cluster, which is a collection of molecules together with one or more ions that is viewed as being an entity intermediate between the gas and condensed phases, can be produced in abundance in an Electrospray.[49-70] The propensity to produce charged clusters in an Electrospray has been recently exploited by Fernandez de la Mora in his experiments that involved studies of neutral molecule clustering around small charged clusters.[71] Again, this is reminiscent of the classic gas-phase ion molecule clustering studies discussed above. Protein clusters produced by an Electrospray have also been observed in the gas phase [73-76] and also after their deposition onto a surface.[77]

Many studies have used acoustic levitation to suspend droplets in air in which protein crystallization was studied.[78-80] One of the investigators, Staffan Nilsson, has claimed that his technique is of general utility because his methodology allows for experiments to be designed to learn of the optimal conditions in which nucleation is initiated while, importantly, each one of his experiments consumes only picoliters of sample solution.[81-83] A company in San Diego has also described a similar approach, with respect to the consumption of only picoliters of sample solution per experiment.[84] This company did not utilize levitation, but they were using similar droplet generators (ie. ink-jet style droplet-on-demand generators) and that was the foundation for their automation of the experimentation work needed to define optimal conditions for protein crystallization.[84]

Other groups have used a combination of acoustic and electrostatic forces to levitate a droplet, and in some reports, deliberately caused the levitated droplet to rotate slowly in an effort to simulate a space environment (i.e. the condition of microgravity) while proteins were allowed to crystallize.[85-91] Chung and co-workers have described at length how the net elementary charge in their levitated droplets was restricted to the surface of the levitated droplet.[86-87] To them, this was an important feature of their nucleation medium because they felt that a “homogeneous interior” volume of the droplet, and specifically that its net neutral charge was an important factor in allowing proteins to nucleate, and then grow into crystals. These authors described the volume of liquid surrounding the “homogeneous interior” as containing the net charge in the droplet. These authors gave the impression that the electric field on the surface of the levitated droplet (due to the net excess charge carried by the droplet) would (or could?) interfere with protein crystallization. However, it appears that the mass-to-charge ratio of the droplets levitated using a combination of acoustic and electric forces in the studies by Chung and co-workers were, well above the threshold necessary to observe effects on the nucleation of a solute as described below.

Another set of groups have been using optical trapping and its forces to promote crystal growth, and they can also transfer those crystals to a growth solution (ie. seeding).[92] Other groups are simply using the intense electric fields of a focused laser beam to induce nucleation.[93-96] There is one report of the use of laser ablation for crystal growth, and that report was applied to diamond growth.[97]

Of possible relevance to the crystallization of proteins are the types of interactions between proteins and various substrates. Theories of protein crystal growth are now emerging in which a protein, charged because its functional groups are protonated or deprotonated (ie. —NH₂OR —COOH respectively) at the condition of the nucleation medium being studied, require the co-precipitation of counter-ions in the growing crystal to maintain near-zero, or zero, electrical neutrality in the crystal.[98] The electrical considerations of protein crystal growth have also begun to be explored in both experiment and theory.[99-102] Electrostatic forces in crystal alignment have been characterized for liquid crystals.[103] and micro-ion disposition.[104] These findings could be of relevance to protein crystal growth.

A well-known phenomenon that deserves mention here is that the interaction of intact cells with substrates has been studied in detail. Most studies have concluded that electrostatic interactions, at least for cell-substrate interactions, are important.[105-110] The sorption of organic compounds onto inorganic compounds has also be studied extensively.[111, 112] and the study of the range of chemical reactions catalyzed on such surfaces remains an active area of research.[113-123] A related discipline is biomineralization, which involves the use of organic compounds such as proteins to promote or catalyze the formation of solids of inorganic compounds.

Recent observations in the inventors' laboratory suggest that the net excess charge in a nucleation medium (i.e. a reaction vessel such as a levitated droplet) is an experimentally accessible variable that does in fact affect the magnitude of the barrier for nucleation in the condensed phase. These finding are described in detail below.

### SUMMARY OF THE INVENTION

In accordance with the invention, a method of controllably inducing nucleation of a first solute dissolved in a solution is described. The method includes the steps of providing a primary vessel for containing said solution; applying an induction potential to said primary vessel such that said solution acquires a net charge; and causing ion-induced nucleation of at least some of said first solute in a condensed phase.

The step of causing ion-induced nucleation may comprise maintaining the surface charge density of said primary vessel above a threshold value and/or maintaining the mass-to-charge ratio of said primary vessel below a threshold value. Ions in the vessel in excess of any counterions induce heterogeneous nucleation of the solute.

In one embodiment of the invention the primary vessel may be wall-less. For example, the primary vessel may be a droplet. The surface charge density may be maintained above the threshold amount in an outer portion of the droplet at an air/droplet interface. After an induction potential has been applied to the droplet, it may be levitated. Many different means of levitation may be employed, such as an electrodynamic balance. The solution may comprise a surface tension modifier to inhibit Coulomb explosion of the droplet.

In other embodiments the vessel may be a droplet in combination with a surface. The primary vessel may comprise a portion of a conduit holding the solution. For example, the conduit may be a capillary.

The ion-induced nucleation may cause formation of one or more nuclei. Volatile solvents in the solution are allowed to evaporate to yield a residue comprising the one or more nuclei. Evaporation of the volatile solvent(s) may have the effect of increasing the concentration of the first solute in the vessel. At least some of said nuclei may be used to promote crystallization of the first solute. The method may further comprise the step of delivering the nuclei to a target location. The target location may be a substrate adapted to receive the nuclei. For example, a portion of the solution comprising the nuclei may be deposited on the substrate. At least some of the nuclei may be delivered from the primary vessel to a secondary vessel for seeding crystal growth in the secondary vessel.

The first solute is preferably a solid dissolved in the solution. For example, the first solute may be an inorganic compound or an organic compound. In one embodiment the first solute may be a biomolecule, such as a protein. Inorganic compounds could include metals, melts and alloys.

In one embodiment of the invention a second solute may be dissolved in the solution in addition to the first solute. The method may comprise selectively precipitating the first and second solutes in order to separate and/or purify the solutes. The method steps may be automated for this and similar purposes. For example, the first and second solutes may be stereoisomers or enantiomers. The invention may also be employed to separate one polymorphic form of a compound from another. In one embodiment the second solute may be a MALDI matrix. The method may result in co-crystallization of the first and second solutes. The method also encompasses precipitates and co-precipitates produced by the method steps.

In one particular embodiment of the invention, the method comprises controllably inducing precipitation of selected solutes dissolved in a solution comprising providing a primary vessel for containing said solution; applying an induction potential to said primary vessel such that said solution acquires a net charge; and selectively causing ion-induced precipitation of at least one of said solutes in a condensed phase. The invention also encompasses a method of controllably inducing crystallization of at least one solute dissolved in a solution, said method comprising providing a primary vessel comprising said solution; controllably imparting a net charge on said solution in a condensed phase to selectively cause ion-induced nucleation of said at least one solute; and depositing crystals derived from said nucleation on a substrate.

### BRIEF DESCRIPTION OF THE DRAWINGS

In drawings which describe embodiments of the invention but which should not be construed as restricting the spirit or scope of the invention in any way,

FIG. 1 is a schematic depiction of apparatus for droplet generation, levitation and deposition in the Applicant's wall-less sample preparation (WaSP) method.

FIG. 2 are sample photographs taken using a CCD camera in conjunction with an optical microscope showing solids of α-cyano-4-hydroxycinnamic acid (CHCA), including visually distinguishable aggregates (A) and nuclei (B) formed by precipitation of a dissolved solid. Solids in these figures were formed and retained in glycerol, a liquid of low volatility and high viscosity.

FIG. 3 are photographs of deposited glycerol droplets that were generated and levitated under the same conditions with the exception of the induction potential. The induction potential was 100, 150 and 200 V for photographs A, B and C respectively. The actual number of crystals of CHCA in each of the residues, and particularly in FIG. 3C, are not all in focus in these single pictures as the depth of view using the microscope optics was smaller than the depth of the glycerol residue in which the CHCA crystals were dispersed. The average size in micrometers of the residues for the conditions used were, A: 57.5±8.8, B: 49.3±4.6, and C, 57.5±6.3.

FIG. 4 is a graph showing the number of nucleation crystals of CHCA in levitated glycerol residues as a function of the DC potential applied to the induction electrode. The residues of the levitated droplets were viewed using an optical microscope and the number of nucleation sites counted manually. The legend indicates the three symbols used to plot the number of crystals within each of the size ranges (diameter of crystal) counted in each droplet.

FIG. 5 is a photograph of a glycerol droplet residue in which there was a single crystal of CHCA. The length of the crystal was 21 μm.

FIG. 6 are photographs of CHCA solids (aggregates and crystals) created without (A, B, and C) and with (D) seeding using nuclei formed in a reaction vessel with net charge (i.e. a levitated droplet).

FIG. 7 are schematic view similar to FIG. 1 showing the Applicant's wall-less sample preparation (WaSP) method employing an electrodynamic balance to prepare and deliver μm-sized charged droplets to a target for subsequent analysis.

FIG. 8 is an enlarged illustration of a droplet generator for creating standard glycerol droplets carrying net excess charge.

FIG. 9 are graphs showing: (A) The average net excess charge carried by a single droplet as a function of the amplitude of the DC potential applied to the induction electrode. All droplets were dispensed with ±10 V applied to the droplet dispenser piezoceramic and each data point is the average charge per droplet of 100 droplets dispensed at 100 Hz. (B) The total charge delivered to the target plate as a function of the number of droplets deposited onto the plate. The single droplets were dispensed at 0.5 Hz with ±30 V applied to the droplet dispenser piezoceramic. The amplitude of the DC potential applied to the induction electrode is noted next to each set of data points.

FIG. 10 are digital images of pairs of glycerol droplets levitated in the EDB while the DC potential applied to the induction electrode was maintained at 100 V while ACₜᵣₐₚ was set to (A) 1600 V_{0-P} and (3) 1000 V_{0-P}. Droplet 1 was created with a higher m/z relative to droplet 2 by applying an IP_{f} of 50 V and 100 V, respectively. Also included is a depiction of the experimental apparatus to aid in the orientation and conditions under which the images were collected.

FIG. 11 are illustrations of steps defining two modes of operation of a WaSP-based charged particle filtering device for single particle delivery to a target.

FIG. 12 are photographs of eight MALDI sample spots created by depositing 4 aliquots of 1 μl CHCA/renin mixture onto the MALDI plate (A-D) grounded and (E-H) at +500 V DC. The white box denotes the area of each sample spot that was magnified to 10 times (bottom rows of images).

FIG. 13 is a graph showing the total renin ion count (the sum of all ion intensity from 1760-1767 Da) measured from the sample spots prepared with a grounded and +500 V DC MALDI plate during crystallization.

FIG. 14 are graphs showing the effect of a DC potential applied to the MALDI plate during matrix crystallization on the ion intensity. Eight 1 μL aliquots of (A) a cytochrome C/sinapinic acid mixture or (B) a renin/α-cyano-4-hydroxycinnamic acid mixture were deposited onto a MALDI plate that was grounded (open circles) or had +5000 V DC applied to it (filled squares). Each data point represents the average of 800 laser shots collected as sets of 10 shots on 10 random positions for each of the eight sample spots.

FIG. 15 is MALDI-TOF-MS spectra of ACTH from sets of 10 droplets, each created at 1 Hz using an induction potential of 100 or 200 V, and levitated in an EDB for 5 minutes. Each spectrum is the average of 8 laser shots acquired using a laser power setting of 4.47×10⁻⁴ J/shot. The spectrum identified by “sweet spot” denotes a spectrum corresponding to well-formed CHCA crystals in the residues of droplets that had been created at 100 V.

FIG. 16 is MALDI-TOF-MS spectra of ACTH from sets of 10 droplets, each created at 1 Hz using an induction potential of 90 or 170 V, and levitated in an EDB for 5 minutes. Each spectrum is the average of laser shot numbers 1-32, acquired using a laser power setting of 4.47×10⁻⁴ J/shot.

FIG. 17 is MALDI-TOF-MS spectra of ACTH from sets of 10 droplets, each created at 1 Hz using an induction potential of 90, 120, or 170 V, and levitated in an EDB for 5 minutes. The spectra are (A) the average of laser shot numbers 1-32, and (B) the average of laser shot numbers 33-64. All spectra were acquired using a laser power setting of 4.47×10⁻⁴ J/shot.

FIG. 18 is MALDI-TOF-MS spectra of ACTH from sets of 10 droplets, each created at 1 Hz using an induction potential of 90, 100, 160, or 170 V, and levitated in an EDB for 5 minutes. The spectra are (A) the average of laser shot numbers 1-32, and (B) the average of laser shot numbers 33-64, and (C) the average of laser shots 65-98. The spectra were acquired using a laser power setting of (A) 3.31×10⁻⁴ J/shot, (B) 4.47×10⁻⁴ J/shot, and (C) 3.31×10⁻⁴ J/shot.

FIG. 19 is a graph and inset photographs showing ACTH ion signal intensity detected from 32 laser shots directed at two discrete sample spots prepared from 10 IP_{f,170V} and 10 IP_{f,90V} droplets, respectively. To collect each mass spectrum the laser was directed at the sample spot and held stationary while it was pulsed because all ten droplets that formed each sample spot fit within the laser spot diameter. The data constituting each mass spectrum were subjected to a boxcar average of five points for smoothing purposes and no background subtraction or X-Y offset was performed. Inset: Images of single glycerol droplet residues created from 10 IP_{f,90V} (a-d) or IP_{f,170V} droplets (e-h), levitated in the EDB for 2 min and deposited onto a glass slide. The solids (ACTH cocrystallized with CHCA) inside the deposited residues were formed while the droplets were levitated.

FIG. 20 are histograms showing filtering of levitated droplets as a function of their m/z by ejecting them one at a time from an EDB. The mean deposition potential (DP) measured for droplets created at different induction potentials (IPf) and levitated simultaneously for a population of (a) 5 droplets (10 replicates) and (b) 10 droplets (5 replicates, population consisted of 2 droplets at each IPf). Presentation of this data as a histogram of the number of droplets deposited at 15 V intervals created a data set that could be construed as a mass spectrum of the levitated charged droplets (left axis).

FIG. 21 are a series of graphs and photographs showing measurement of differential chemical processing that occurred within droplets whose net charge was 140 or 290 fC. (a) Histogram of the number of droplets deposited at 15 V DP intervals for a population of 40 droplets, 20 IP_{f,50V} and 20 IP_{f,200V}. Inset: Image of droplet population levitated in EDB prior to ejection. (b) Light microscopy of the codeposited droplets before (I=20 IP_{f,200V} droplets, 2=20 IP_{f,50V}) and the same spots respectively after vacuum drying (3, 4). The bottom right of the images show a sample spot created from 0.25 μL delivered by pipette (5). Sample spot regions are outlined by dotted white circles that are intended to be guides for the eye. (c) MALDI-MS characterization of the solids formed in the residue labeled as 3. The measured peptide monoisotopic masses are labeled, and the quantity of the following peptides per spot were i=bradykinin (220 amol), ii=angiotension Π (220 amol), and iii=angiotension I (22 amol). No peptide ion signals were detected from sample spot 4.

FIG. 22 are graphs showing the mean monoisotopic [ACTH+H⁺] peak height from 50 laser shots fired at sample spots created from (a) 12 IP_{f,90V} droplets (3 nL) (open squares) or 12 IP_{f,170V} droplets (filled squares) and (b) 1.00 μL deposited by pipette on a stainless steel MALDI plate that was grounded (open circles) or had +500 V DC applied to it (filled circles). Ion abundances were measured at increments of the neutral density filter that determines the laser irradiation. At the 20 and 100% settings for laser irradiation energy, the energy per pulse was measured to be 30 and 121 μJ, respectively.

FIG. 23 are representative images of residues of levitated droplets that had (A)-135 and (B)-325 fC of net charge. These droplets were dispensed from a starting solution containing 285 mM NaCl in 97:3 water:glycerol. NaCl precipitate curvature in FIG. 23B, indicated by the dotted line, were similar and indicate that crystal nucleation and initial growth took place in the droplet-air interface of the levitated droplet. (C) Residues of droplets dispensed from a starting solution having 69 mM NaCl in 97:3 water:glycerol that had either −135 or −350 fC of net charge as a function of their levitation time.

FIG. 24 is a graph and photographs showing (A) Percentage of levitated droplet residues that contained NaCl precipitates having curved morphology as a function of the dc induction potential during the droplet dispensing event. The top x-axis indicates the levitated droplet m/z [(mean±standard deviation) n=5 groups of 10 droplets each]. The insets show precipitates having (B) cubic and (C,D,E) regions of curvature. The precipitates in the images identified as B, C, D, E were observed in the residues of levitated droplets that had been formed at dc induction potentials (B) 140 V, (C) 160 V, (D) 180 V, and (E) 200 V, respectively.

FIG. 25 are schematic representations of ion-induced nucleation in the diffuse layer at a droplet-air interface of a droplet having net charge. The electrical potential at the droplet-air interface due to the net charge is depicted in a darker shade, and zero electric potential in the droplet core in a lighter shade. Counterions with net positive charge are depicted as well as anions and ion_{NEC}. (a) A representation of counterions in the vicinity of each ion_{NEC}. (b) Ion-induced nucleation at one of the sites of ion_{NEC}. The arrows indicate re-distribution of ions_{NEC} for the specific case of a cation combining with a site of net negative charge. (c) Ion-induced growth of the embryo in the droplet-air interface.

FIG. 26 are images acquired by optical or fluorescence microscopy of (A, B) droplets and (C, D) main residues following Coulomb explosion. (A) One droplet dispensed at each (i) 100V, (ii) 150V, and (iii) 200V dc applied to the induction electrode and using the starting solution consisting of 320 nm fluospheres in 97:3 water:glycerol. These droplets were briefly levitated and allowed to fall to the target and land at a location along the y-axis proportional to its m/z. (B) Same as in (A), but the target plate was translated in the direction of the x-axis between droplet dispensing events. (C) Droplets dispensed from a starting solution consisting of either 7.5 or 37.5 mM NaCl in water with a dc induction potential of 200V, and those droplets were individually levitated until they underwent Coulomb explosion, and the resultant main residues landed on the target at the locations indicated by the black arrows. The nature and number of the NaCl precipitates formed in these droplets (that had no glycerol present) are apparent in insets identified as i-iv. Residues were not completely dry upon deposition. (D) Droplets dispensed from a starting solution consisting of either 21 or 320 nM fluospheres in water with a dc induction potential of 200V, and those droplets were individually levitated until they underwent Coulomb explosion, and the resultant main residues landed on the target at the locations indicated by the white arrows. The main residues observable in insets (C, D) were generated from droplets dispensed from different starting solutions, but because the dispensing events used the same induction potential (200 V dc), the original droplets therefore had initial volumes and net charge that were the same within experimental error, the target plate was translated in the x-direction between each droplet dispensing event, and the dotted straight lines are intended as guides for the eye to indicate the mean location of deposition of the main residues as a function of solutes concentration in the starting solution, and the direction of the y-axis vector indicates the relative location of deposition of main residues of high to low m/z.

FIG. 27 is a series of graphs showing: a, Percent occurrence of ion-induced nucleation as a function of induction potential in droplets with various sizes. Empty squares, filled circles, filled squares, and empty circles denote droplets created with 1, 2, 3, and 4% glycerol in the starting solutions. b, c, The volume charge density (b) and the surface charge density (c) at the onset of ion-induced nucleation as a function of the droplet's radius. d, Percent occurrence of ion-induced nucleation as a function of induction potential for droplets with different glycerol-water composition. Filled circles, empty circles, and filled squares, denote 31, 10, and 3% water by volume, respectively. e, the surface charge density at the onset of ion-induced nucleation as a function of the droplet's viscosity. f, The change of mass-to-charge ratios and sodium chloride concentrations as a function of time for two populations of droplets having −135 fC and −350 fC of net charge, respectively. Grey line, black line, and dashed line denote the mass-to-charge ratios for the population of droplets with −135 fC and −350 fC and the concentration of sodium chloride, respectively. g, The average number of crystals per droplet as a function of time. Grey and black bars indicate data collected from population of droplets with −135 fC and −350 fC, respectively.

FIG. 28 are representative images of a-c, Representative images of á-cyano-4-hydroxycinnamic acid (CHCA) solids and their abundance that had formed during the period of time droplets were levitated. The net charge on the droplets was a, −140 fC, b, −235 fC and c, −325 fC. d, Counts of CHCA crystals as a function of the induction potential applied. Black bars, Grey bars, and shaded bars denote crystals of the size >3.5 μm, between 1 μm and 3.5 μm, or <1 μm, respectively. e, f, Representative images of 2,4,6 trihydroxyacetophenone crystals formed in droplets that had −140 fC (e) or −325 fC (f). The solvent was allowed to completely evaporate for better visualization. The observable morphology of the crystals did not change as a result of this experimental manipulation.

FIG. 29 are graphs showing a, Percent droplets were precipitation of L-serine (squares) and D-serine (triangles) occurred as a function of % relative humidity. Droplets were levitated at 100 V IP_{f} (empty squares and empty triangles) or 200 V (filled squares and filled triangles). b, Weight of precipitated material from 1 mL mixtures solution of D- and L-serine with different % composition. c, Percent droplets were precipitation of D- and L-serine solution mixtures occurred as a function of % L-serine in the solution. d, The AC potential (relative) required to center glycerol droplets with known mass-to-charge ratios as a function of induction potential. E, Volume-to-charge ratios of droplets with −325 fc (+200 V induction potential) as a function of the relative AC potential required to center them in the null point of the EDB.

### DETAILED DESCRIPTION OF THE INVENTION

Throughout the following description specific details are set forth in order to provide a more thorough understanding of the invention. However, the invention may be practiced without these particulars. In other instances, well known elements have not been shown or described in detail to avoid unnecessarily obscuring the present invention. Accordingly, the specification and drawings are to be regarded in an illustrative, rather than a restrictive, sense.

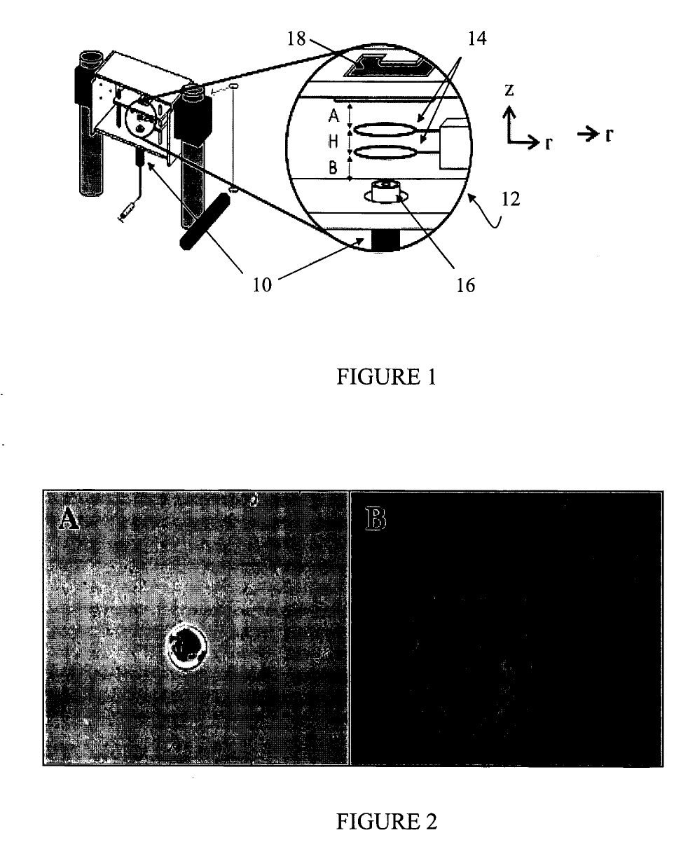

The inventors have previously described various methods for levitation and manipulation of charged droplets. Apparatus used by the inventors for droplet levitation is described in detail in the literature and in Applicant's prior international application No. PCT/CA01/01496 filed 23 Oct. 2001 and entitled “Method and Apparatus for Producing a Discrete Particle” (WO 02/035553 A3), the disclosure of which in hereby incorporated by reference.[8, 124]. Related subject matter is also described in Applicant's prior international application No. PCT/CA04/000242 filed 24 Feb. 2004 and entitled “Formation of Closely Packed Microspots and Irradiation of Same” (WO 2004/075208 A3), the disclosure of which is hereby incorporated by reference. As shown in FIG. 1, the levitation apparatus comprises a droplet generator 10, an electrodynamic balance (EDB) 12, including spaced-apart ring electrodes 14, an induction electrode 16 and a target sample plate 18. Sample plate 18 may, for example, be a MALDI plate located at a position remote from electrodynamic balance 12.

Particulars of the droplet generation, levitation and deposition processes are described in detail in the experimental section below. This approach is sometimes referred to as “wall-less sample preparation” (WaSP). Briefly, a small amount of a starting solution of known composition is initially loaded in droplet generator 10. The starting solution may contain one or more solutes of interest (typically present as dissolved solids). The starting solution also typically includes one or more volatile and non-volatile solvents. A quantity of solution is ejected from the nozzle of droplet generator 10 in the vicinity of induction electrode 16 to form an initial droplet. As the initial droplet emerges from droplet generator 10, a DC potential applied to induction electrode 16 induces a net charge on the droplet. The initial droplet may then by deposited on to a substrate directly or injected into electrodynamic balance 12 and levitated there for a period of time.

Typically volatile solvents present in the initial droplet evaporates quickly to yield a residue of the initial droplet. The residue is comprised of solvents and solutes of lower volatility. Coloumb explosion of the initial droplet or droplet residue, a process that ordinarily causes a droplet with net charge to fragment, can be avoided by including in the starting solution a compound having high surface tension, such as glycerol.

Droplet residues having net charge may be suspended in the EDB 12 for a desired length of time (which can vary from a few milliseconds to several hours), for example to allow initiation or completion of a desired chemical reaction, and may then be controllably delivered to a target location, such as substrate remote from the EDB. As explained above, the substrate on which the droplet residue is deposited may be a MALDI plate 18 in one embodiment of the invention. As described in Applicant's prior PCT application (WO 2004/075208 A3), the Applicant's method enables droplet residues, or portions thereof, to be deposited on the target substrate as microspots in a spacially precise manner. For example, different microspots may be deposited on the substrate in very close proximity to one another.

The deposited material may then be further analyzed or characterized by various different means, as described further below. For example, the microspots may be irradiated and the resulting ions detected by mass spectrometry, such as time of flight mass spectrometry. The deposited material may also be characterized using an optical microscope or the like.

The present invention had its genesis in the unexpected observation that solutes (i.e. dissolved solids) present in the starting solution had a greater propensity to nucleate and form crystals when the reaction vessel (e.g. a levitated droplet) was subjected to an induction potential of larger magnitude such that the reaction vessel had a net excess charge. In particular, it was initially determined that the energy barrier for dissolved solids to nucleate is affected by the mass-to-charge ratio of the reaction vessel. A reduction in the mass-to-charge ratio of the vessel causes the barrier for nucleation to decrease. More recently, the inventors have determined that the most relevant experimental factor appears to be the surface charge density of the vessel. That is, there appears to be a linear relationship between the surface charge density of the vessel (e.g. a droplet) and the onset of nucleation. As described in detail below, in droplets having a high surface charge density (i.e. a low mass-to-charge ratio), a large number of small nuclei are observed. In contrast, in droplets having a lower surface charge density (and a relatively higher mass-to-charge ratio), very few or no nuclei were observed in residues of the droplets. When solids did form in those levitated droplets, they tended to be aggregates rather than nuclei. These observations suggest that reaction vessels that have an appropriate surface charge density (and mass-to-charge ratio) promote the formation of nuclei, and possibly catalyze the nucleation.

The sample pictures shown in FIG. 2 show solids of α-cyano-4-hydroxycinnamic (CHCA) in deposited droplet residues taken with a CCD camera mounted on the trinocular head of an optical microscope. This figure illustrates how CHCA nuclei are visually distinguishable from aggregates. In FIG. 2 nuclei of CHCA appear as transparent white solids with sharp edges, aggregates of CHCA appear as opaque globular solids and crystals of CHCA appear as clear solids. As will be apparent to a person skilled in the art, crystals of CHCA are grown on nuclei of CHCA, but not on aggregates of CHCA.

The inventors have previously measured the distribution of an organic dye cation (Rhodamine 6G) within NaCl that results when a droplet with net charge is allowed to dry while levitated.[125] From that work, a measure of the thickness of the surface layer that contains the net charge on a droplet with net charge was obtained. That data indicates that droplets with net charge can be described as imperfect conducting spheres. The thickness of the surface layer was determined to be several micrometers in thickness which is much larger than the expected thickness of an electric double layer. This suggests that the surface volume is quite different in its chemical and physical description than the interior of the droplet with net charge. As explained above, the ion induced nucleation phenomenon which varies with the surface charge density (and the mass-to-charge ratio of the reaction vessel) may be as a result of the electric field at the interface between the surface and bulk-like interior volumes within these droplets, plus the fact that electrical neutrality is not maintained in these droplets with net charge. The presence of an electric field (i.e. increased net charge, so reduced mass-to-charge ratio) appears to influence the magnitude of the thermodynamic barrier leading to nucleation of a solute. The inventors believe that the electric field causes the alignment of molecular dipoles of the solutes in the droplet, and that effects a reduction in their internal energy. As explained below, the inventors have demonstrated lowered solubility of some solutes as a function of the net charge of the reaction vessel.

As will be appreciated by a person skilled in the art, many reactions occur as a result of a charge imbalance within or between reaction vessels that have zero net charge. By contrast, as explained above, the present invention relates to reaction vessels having “net charge” or “net excess charge” which are suitable for promoting ion-induced nucleation. The term “ion” as used herein refers to atoms or molecules that carry charge. The terms “net charge” and “net excess charge” as used herein refers to the presence of ions in a vessel of a single polarity that are in excess of the counterions of opposite polarity present within the same vessel. As used in this patent application the reaction “vessel” may simply consist of a droplet of a solution having a net charge, or may alternatively consist of a droplet together with a supporting surface. For example, a “vessel” may consist of a droplet deposited on a surface or held within a container, such as a capillary or portion thereof. Most references herein to the terms “reaction vessel” and “nucleation vessel” refer to a droplet wherein nucleation is induced, but the invention is not restricted to that embodiment.

The inventors' findings can be exploited as described herein in order to elicit selective control over the induction of nucleation and subsequent crystallization of target solutes of interest in the condensed phase. The inventors anticipate that this ion induced nucleation phenomenon, in reaction vessels having a desirable surface charge density, is likely to be general for all dissolved solids, ranging from inorganic compounds, to low and high molecular weight organic compounds, including proteins and other molecules. For example the present invention can be used to selectively crystallize a target solute or to separate different solutes from one another based on their propensity to nucleate at different reaction conditions. The different solutes could constitute different compounds or different stereochemical forms of same compound. The invention could also be exploited to controllably select or separate polymorphic forms of a compound (which may often have very different biological activity). The crystals derived from the process could be the subject of further analysis, characterization or manipulation, for example as a prepared sample material for MALDI-TOF MS. Examples describing the controlled induction of nucleation and crystallization of various compounds are described in detail below.

### EXAMPLES

The following examples will further illustrate the invention in greater detail although it will be appreciated that the invention is not limited to the specific examples.

The following description of experimental details and experimental results is presented in multiple parts. Example 1.0 describes an observation regarding the nucleation of an organic compound (CHCA) in a levitated droplet. The reproduction of that observation led to the realization that the net charge of the reaction vessel (e.g the mass-to-charge ratio of the vessel) influences the nucleation of a solute. In this example the reaction vessel is a levitated droplet. Example 2.0, which summarizes experiments performed independently of Example 1.0, describes measurement of droplet mass and net charge, and the filtering of droplets with net charge (i.e. the reaction vessels in the work described in section Example 1.0) as a function of their mass-to-charge ratio. Example 2.0 also describes the effect of allowing a droplet dispensed from a micropipette to be deposited on to a biased plate. Example 3.0 describes MALDI matrix and analyte compound co-precipitates. Example 4.0 describes promotion of CHCA and peptide cocrystallization within levitated droplets having net charge. Example 5.0 describes the measurement of chemical parameters, such as promotion of NaCl precipitation, in droplets with net charge which were not allowed to undergo Colulomb explosion. Example 6.0 describes ion-induced precipitation of NaCl, CHCA, THAP and samples of D and L serine in levitated droplets possessing net charge.

### Example 1.0

### 1.1 Nucleation Studies of Dissolved Solids in Levitated Droplets with Net Charge

### 1.1.1 Experimental Apparatus and General Procedure for Levitation of Droplets with Net Charge

Apparatus used by the inventors for droplet levitation is described in detail in the literature and in Applicant's prior international application No. PCT/CA01/01496 filed 23 Oct. 2001 and entitled “Method and Apparatus for Producing a Discrete Particle” (WO 02/035553 A3), the disclosure of which in hereby incorporated by reference.[8, 124] As described generally above and as shown in FIG. 2, the levitation apparatus comprises a droplet generator 10, an electrodynamic balance (EDB) 12 including spaced-apart ring electrodes 14, an induction electrode 16 and a target sample plate 18. Sample plate 18 may be a MALDI plate located at a position remote from electrodynamic balance 12.

Droplet generator 10 used in the following examples is a commercially available ink-jet style, droplet-on-demand generator (Microfab, Plano, Tex., USA, e.g models MJ-AB-01-60 and MJ-AB-01-40) which requires as little as 2 μL of starting solution to function. As explained below, the starting solution may include both volatile and non-volatile solvents and solutes, including the solutes targeted for analysis. Each droplet is generated by applying a time-dependent waveform to an annular shaped piezoelectric crystal bonded to the outside of the glass capillary of the droplet generator 10. The size of the piezoelectric crystal change and the time dependence of that crystal size change effected by the amplitude and temporal characteristics of the AC waveform respectively, create a pressure wave inside the glass capillary of the droplet generator 10. In turn, that pressure wave forces a volume of liquid out of the nozzle of the droplet generator. While that volume of liquid is emerging from the nozzle, it takes on the form of a jet, and the DC potential applied to the induction electrode 16 induces a net charge onto that jet of liquid such that when the momentum imparted into the jet causes that jet to separate from the nozzle and the jet collapses into a droplet, that droplet has a net charge. The droplet generator is positioned such that each droplet flies into the center of an electrodynamic balance (EDB), where it can be trapped and levitated, provided the electric field and the droplet's mass-to-charge are appropriate.

As explained above, the starting solution loaded into the droplet generator, is typically prepared by mixing several volumes of different stock solutions together. Stock solutions are used because one or more of the target solutes may require dissolution in a particular solvent. By way of non-limiting example, a starting solution with a total volume of 400 μl was prepared by the addition of: i) 60 μL of a solution containing 20% glycerol to distilled deionized water by volume; ii) 40 μL of acetone; iii) 40 μL of a solution saturated in α-cyano-4-hydroxycinnamic acid (CHCA) in 50:50 v:v acetonitrile:0.1% TFA in distilled deionized water; iv) 180 μL of acetonitrile; and v) 80 μL of distilled deionized water. In another example, a starting solution with a total volume of 400 μl was prepared by the addition of: i) 60 μL of a solution containing 20% glycerol to distilled deionized water by volume; ii) 40 μL of acetone; iii) 40 μL of a solution saturated in α-cyano-4-hydroxycinnamic acid (CHCA) in 50:50 v:v acetonitrile:0.1% TFA in distilled deionized water; iv) 180 μL of acetonitrile; and v) 80 μL of distilled deionized water. In yet another example, a starting solution that contained a different volume of the saturated solution of CHCA was prepared from: i) 60 μL of a solution containing 20% glycerol to distilled deionized water by volume; ii) 40 μL of acetone; iii) [40+(x)] μL of a solution saturated in α-cyano-4-hydroxycinnamic acid (CHCA) in 50:50 v:v acetonitrile:0.1% TFA in distilled deionized water; iv) [180−(0.5×)] μL of acetonitrile; and v) [80−(0.5×)] μL of distilled deionized water. As will be apparent to a person skilled in the art, the identity and concentration of solutes and solvents in the starting solution may vary depending upon the desired analysis.

Induction electrode 16 applies a net charge to a droplet of the starting solution as it emerges from droplet generator 10. By way of example, induction electrode 16 may be made of copper shaped in a disk with a 4 mm diameter hole cut in its center. As shown in FIG. 2, the nozzle of droplet generator 10 is centered over the hole in induction electrode 16 such that the vertical separation between the nozzle and the induction electrode is ˜2 mm. As explained above, the induction electrode 16 placed near the orifice of the droplet generator 10, and the DC potential applied to that electrode, is used in conjunction with the time-dependent waveform applied to the droplet generator 10 to make a droplet that has mass and net charge. As explained below, the induction potential applied by electrode 16 can be varied depending upon the experimental parameters and desired results.

In use, immediately upon formation of the droplet, solvents in that droplet begin to evaporate. For example, one or more of the solvents in the droplet are typically of low viscosity and high vapor pressure. These solvents rapidly evaporate, usually within seconds after formation of the droplet. This solvent evaporation is occurring while the droplet flies to the EDB 12 and continues while it is levitated in the EDB. The starting solutions used in these examples typically incorporate glycerol at a few percent by volume. Glycerol is a solvent of high viscosity and low vapor pressure to avoid Coulomb explosion and enable droplet lifetimes on the order of hours (although in these examples the droplets were often levitated for only a few minutes). The physical and chemical description of the levitated droplet after the rapid evaporation of its volatile solvents is a function of the starting solution composition, the conditions used for droplet generation, whether or not Coulomb explosion occurred, and the environmental conditions such as temperature and humidity in the chamber in which levitation is performed. At this stage in the process the levitated droplet is sometimes referred to as a droplet “residue” in the sense that its composition, though known, is at this stage quite different than the composition of the starting solution prior to evaporation of solvents.

Following a period of levitation, which can range from a few milliseconds to hours, the droplet residue is typically deposited onto a substrate remote from the EDB by adjustment of the electric field in the EDB 12, such as the MALDI plate 18 (FIG. 1). At this point in an experiment, the inventors are able to characterize the composition of the deposited droplet residue for solids using instrumental techniques. For example the deposited residue may be characterized using MALDI-TOF mass spectrometry or visualization using an optical microscope. This entire procedure which has been described in serial (i.e. creation, levitation, and deposition of a droplet) can be made parallel with respect to a sequential and rapid injection of multiple droplets into the EDB 12, levitating all of them for the duration of a pre-determined levitation period and then causing their sequential deposition. Furthermore, multiple droplet residues levitated in the EDB can be co-deposited onto a single location on a target, or deposited individually, wherein each droplet could be caused to deposit at a different location on the target to form an array of deposited droplet residues. As will be appreciated by a person skilled in the art, this process may be automated.

### 1.1.2 Results Concerning the Nucleation of CHCA as a Function of the Mass-to-Charge of the Levitated Droplet

### Experiment 1

In a first experiment, the number and type of solids in the residues of levitated droplets that had been produced using a positive induction potential (i.e. the droplets had net negative charge) has been observed to vary with the magnitude of the DC potential applied to the induction electrode. The results of this experiment in which the only variable was the DC potential applied to the induction electrode 16 are presented in Table 1 below. (Because this was one of the first reproducible observations of dissolved solids forming in levitated droplet residues as a function of the levitated droplet's mass-to-charge ratio, the inventors did not segregate precipitated solids on the basis of whether they were aggregates or crystals).

TABLE 1

| | Induction Potential (Volts) | | |
|---|---|---|---|
| Droplet Number | 100 | 150 | 200 |
| 1 | 0 | 2 | 1 |
| 2 | 0 | 7 | >9 |
| 3 | 1 | 2 | 7 |
| 4 | 0 | 3 | 0 |
| 5 | 0 | 0 | 0 |
| 6 | 0 | 0 | ~45 |
| 7 | 3 | 0 | 0 |
| 8 | 0 | 3 | 5 |
| 9 | 1 | 5 | 2 |
| 10 | 1 | 2 | 2 |
| 11 | 5 | 5 | 8 |
| 12 | 0 | 2 | 1 |
| 13 | 2 | 3 | 6 |
| 14 | 4 | 2 | 4 |
| 15 | 2 | 5 | 10 |
| 16 | 0 | 4 | 0 |
| 17 | 0 | 0 | 3 |
| Average | 1 | 4 | 6 |

In this particular experiment, a single waveform was used to generate the droplets and thus the nominal mass of each of the droplets was unchanged within experimental error. Thus the change in the induction potential caused a change in the droplet mass-to-charge ratio.

The composition of the starting solution (total volume=200 μL) was as follows: i) 30 μL of a solution containing 20% glycerol to distilled deionized water by volume, ii) 50 μL of a solution saturated in α-cyano-4-hydroxycinnamic acid (CHCA) in 50:50 v:v acetonitrile:0.1% TFA in distilled deionized water, and iii) 120 μL of distilled deionized water

The data in Table 1 indicate that there was an increase in the number of solids present in the droplet residues as a function of the DC potential applied to the induction electrode 16 at the time of formation of the droplets. A higher DC induction potential resulted in a more CHCA solids in the residues of the levitated droplets.

### 1.1.3 Results Concerning the Nucleation of CHCA as a Function of the Mass-to-Charge of the Levitated Droplet

### Experiment 2

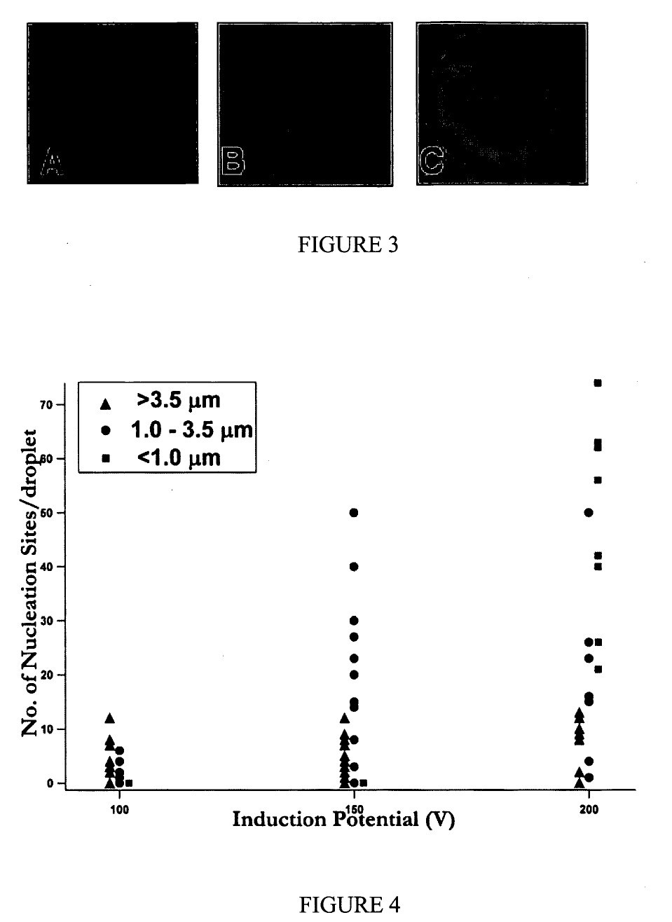

Another nucleation experiment was performed as shown in FIG. 3. In FIG. 3, three pictures of sample residues of levitated glycerol droplets are shown. Each of those residues was created from a single starting solution (total volume=400 μL) that consisted of i) 60 μL of a solution containing 20% glycerol to distilled deionized water by volume, ii) 40 μL of acetone, iii) 100 μL of a solution saturated in -cyano-4-hydroxycinnamic acid (CHCA) in 50:50 v:v acetonitrile: 0.1% TFA in distilled deionized water, and iv) 150 μL of acetonitrile, and v) 50 μL of distilled deionized water. During this experiment, each droplet was generated from the starting solution using the same waveform applied to the droplet generator 10. This ensured that, within experimental error, the size of all droplets at the instant they were formed were similar. The variable in this experiment was the amplitude of the DC potential applied to the induction electrode 16. (As a result of levitating droplets whose mass-to-charge ratio was different, the amplitude of the AC waveform applied to the ring electrode 14 and the amplitude of the DC potential applied to the target (i.e. the deposition plate 18) were different.

The levitated droplet residues were each deposited after a levitation period of 3-5 minutes in this experiment. The photographs clearly show that the nucleation barrier was affected (i.e. reduced) when the induction potential was raised. The black appearance of globular shaped aggregates are apparent in FIG. 3B (150 V), whereas sharp edged nuclei that are apparent in FIG. 3C (200 V) are whiter and clearer relative to the solids in FIG. 3B.

The number of crystals, and the size of those crystals, from 18 different droplets at each of the induction potentials 100, 150, and 200 V were further characterised using an optical microscope as shown in FIG. 4. Again, the number of small crystals in the levitated droplets increased with an increase in the induction potential. The inventors believe that the cause of the increased nucleation was the increased net charge per droplet. In the generation of droplets of similar size, an increase in the induction potential lowers the droplet mass-to-charge ratio. The higher net charge in a levitated droplet is believed to cause a reduction of the magnitude of the barrier for nucleation of a dissolved solid in that droplet.

Within the crystal growing community, it is known that the full description of the state of a system should include an electrostatic term, but because crystallisation has almost always performed within a net neutral solution, the electrostatic term is very often simply neglected as an experimental variable. The inventors have recognized that net charge is in fact a controllable variable in the nucleation of a solute in the condensed phase.

### 1.1.4 Droplets with Net Positive Charge: Morphological Details of Crystals

### Experiment 3

A nucleation experiment was conducted used a starting solution that was composed of i) 60 μL of a solution containing 20% glycerol to distilled deionized water by volume, ii) 40 μL of acetone, iii) 100 μL of a solution saturated in α-cyano-4-hydroxycinnamic acid (CHCA) in 50:50 v:v acetonitrile: 0.1% TFA in distilled deionized water, iv) 150 μL of acetonitrile, and v) 50 μL of distilled deionized water.

This experiment had two main purposes. The first purpose was to study the effect of crystallization in droplets that contain a net positive charge instead of negative. The second purpose was to gain a better understanding of the morphological details of the crystal surface that were apparent using an optical microscope. From previous results, it was learned that droplets that had been collected on a glass slide were solid and dark in appearance (i.e. visually resembled coffee beans) which made it relatively difficult to discern their surface characteristics using an optical microscope. Hence, it was decided to collect the droplets on a stainless steel MALDI plate that was chrome plated. The surface of this plate had been machined flat and polished. Crystals within the residues of the droplets deposited from the EDB 12 appeared more shiny and defined under the microscope. However, the small crystals that were observable when the droplets had been deposited onto a glass cover slip, were not observable on the stainless steel plate, possibly because there was not sufficient light available to illuminate the solids sufficiently to allow viewing of the smaller crystals. The crystals observed in the deposited droplet residues, and segregated according to their size, are presented in Table 2 below.

TABLE 2

| | Induction Potential (Volts) | | Induction Potential (Volts) | |
|---|---|---|---|---|
| | & Size of Solids | | & Size of Solids | |
| | (μm) −100 V | | (μm) −180 V | |
| Replicate | >3.5 | 1.0-3.5 | >3.5 | 1.0-3.5 |
| 1 | 4 | 0 | 7 | 0 |
| 2 | 0 | 7 | 8 | 10 |
| 3 | 2 | 2 | 1 | 6 |
| 4 | 3 | 0 | 2 | 9 |
| 5 | 1 | 0 | 1 | 5 |
| 6 | 2 | 0 | 8 | 32 |
| 7 | 1 | 0 | 3 | 4 |
| 8 | 3 | 0 | 8 | 36 |
| 9 | 3 | 1 | | |
| 10 | 4 | 0 | | |
| 11 | 4 | 3 | | |
| 12 | 2 | 0 | | |
| 13 | 1 | 3 | | |
| 14 | 1 | 2 | | |
| 15 | 1 | 0 | | |
| 16 | 2 | 0 | | |
| 17 | 3 | 4 | | |
| 18 | 3 | 0 | | |
| 19 | 2 | 0 | | |
| 20 | 4 | 1 | | |
| 21 | 2 | 0 | | |
| 22 | 3 | 1 | | |
| 23 | 3 | 0 | | |
| 24 | 3 | 0 | | |
| 25 | 1 | 3 | | |
| 26 | 3 | 1 | | |
| 27 | 1 | 3 | | |
| 28 | 3 | 3 | | |
| 29 | 1 | 0 | | |
| 30 | 6 | 2 | | |
| 31 | 2 | 3 | | |
| 32 | 1 | 1 | | |
| 33 | 0 | 4 | | |
| 34 | 1 | 0 | | |
| 35 | 2 | 0 | | |
| 36 | 1 | 0 | | |
| 37 | 2 | 0 | | |
| 38 | 1 | 0 | | |
| 39 | 2 | 0 | | |
| 40 | 2 | 0 | | |
| 41 | 1 | 0 | | |
| 42 | 2 | 0 | | |
| 43 | 2 | 0 | | |
| 44 | 2 | 4 | | |
| Average | 2 | 1 | 5 | 13 |

The numbers of precipitates in each glycerol droplet is noted in Table 2. There were 44 replicates performed using an induction potential of-100 V, and 8 replicates performed using an induction potential of −180 V. The crystals sizes observed were categorized as either >3.5 μm in diameter or 1.0-3.5 μm in diameter.

The data presented in Table 2 provides clear evidence that higher relative DC potentials applied to the induction electrode 16 cause the formation of a larger number of crystals relative to when a lower DC potential is used. This experiment was informative to the inventors because this was the first time that the number of crystals of CHCA formed were classified according to their size, as shown in FIG. 4. The large solids, whose size was in the range >3.5 μm in diameter) had an undefined variable shape that appeared to be aggregates, and their surfaces did not shine. The solids whose size was in the range 1.0-3.5 μm in diameter were, in contrast, non-globular in appearance and their surfaces were smoother than the larger solids; these medium size solids appeared more transparent and were likely crystals. The smallest solids observed, <1.0 μm in diameter, are believed to be crystals of CHCA.

### 1.1.5 Growth of Large CHCA Crystal

### Experiment 4

The objective of this experiment was to grow a large crystal of CHCA. As described above, the inventors had previously shown that the mass-to-charge ratio of the reaction vessel can be used to preferentially form crystals of CHCA, rather than aggregates. In this example, we studied the potential to grow a single large crystal in a reaction vessel that was prepared with a mass-to-charge ratio that was in the range in which CHCA nuclei were previously observed to readily form. The levitation period of the nucleation vessel was extended so that the kinetics of crystal growth was not a limiting factor in this experiment.

The starting solution used in this experiment was comprised of: i) 60 μL of a solution containing 20% glycerol to distilled deionized water by volume, ii) 40 μL of acetone, iii) 40 μL of a solution saturated in α-cyano-4-hydroxycinnamic acid (CHCA) in 50:50 v:v acetonitrile:0.1% TFA in distilled deionized water, iv) 180 μL of acetonitrile, and v) 80 μL of distilled deionized water.

Six droplets were created with the induction potential set at +80V. +80 V is a relatively low induction potential, and mass-to-charge ratio of these reaction vessels were relatively high and hence the inventors did not expect to observe CHCA nuclei in this trial. Three of these droplets were levitated for 3 minutes before being deposited, and the remaining three were levitated for a total of 12 minutes before they were deposited. No solids (i.e. no nuclei, aggregates or crystals) were observed in the residues of any of these droplets.

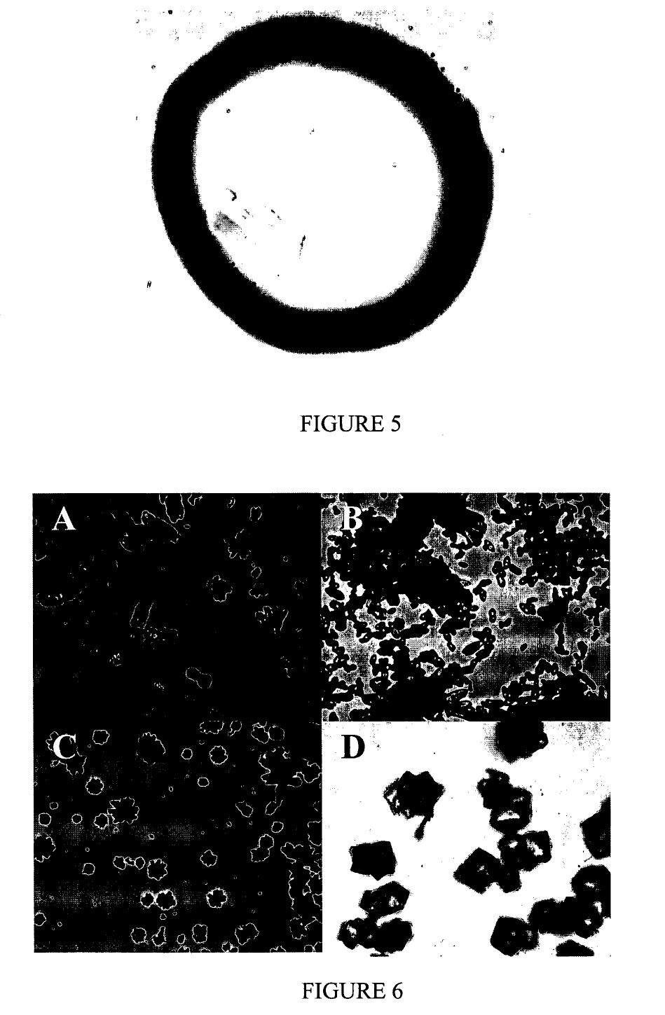

Next, 8 droplets were created at +180 V. +180 V is a relatively high induction potential, so the mass-to-charge ratio of these reaction vessels was relatively low and hence the inventors did expect to observe CHCA nuclei in this trial. Four of these droplets were levitated for 3 minutes, and the remaining 4 droplets were levitated for a total of 12 minutes before they were deposited. In one of the droplet residues that had been levitated for 12 minutes, there was a crystal that had a length of 21 μm (FIG. 5). In the remaining three droplets that had been levitated for 12 minutes, no solids were observed.

### 1.1.6 Formation of Nuclei Versus Aggregates at Different Induction Potentials

### Experiment 5

A further experiment was conducted to test the hypothesis that nuclei versus aggregation of CHCA could be unambiguously controlled by setting the mass-to-charge ratio of the reaction vessel (droplet). Thus, the hypothesis was that the barrier for nucleation of crystals versus aggregates can be reduced in reaction vessels that have a low mass-to-charge ratio.

The starting solution was prepared by the addition of: i) 60 μL of a solution containing 20% glycerol to distilled deionized water by volume, ii) 40 μL of acetone, iii) 40 μL of a solution saturated in α-cyano-4-hydroxycinnamic acid (CHCA) in 50:50 v:v acetonitrile:0.1% TFA in distilled deionized water, iv) 180 μL of acetonitrile, v) 80 μL of distilled deionized water.

The solids observed in the levitated droplet residues were classified as aggregates or nuclei. The nuclei were further differentiated by size and three size ranges were used, as indicated in Table 3 below. Large (>3.5 μm in diameter), medium (1.0-3.5 μm in diameter), and small (<1.0 μm in diameter)

TABLE 3

| | Induction Potential (Volts) and Solid Type or Crystal | | | | | |
|---|---|---|---|---|---|---|
| | Size (□m) | | | | | |
| | 100 V | | | 190 V | | |
| Replicate | agg | 1.0-3.5 | <1.0 | agg | 1.0-3.5 | <1.0 |
| 1 | 1 | 0 | 0 | 0 | 2 | 1 |
| 2 | 1 | 0 | 0 | 0 | 1 | 2 |
| 3 | 2 | 0 | 0 | 0 | 0 | 0 |
| 4 | 0 | 0 | 0 | 0 | 0 | 0 |
| 5 | 0 | 0 | 0 | 0 | 1 | 0 |
| 6 | 1 | 0 | 0 | 0 | 0 | 0 |
| 7 | 0 | 0 | 0 | | | |
| 8 | 0 | 0 | 0 | | | |
| 9 | 0 | 0 | 0 | | | |
| 10 | 0 | 0 | 0 | | | |
| 11 | 0 | 0 | 0 | | | |
| 12 | 0 | 0 | 0 | | | |
| Average | 0.4 | 0 | 0 | 0 | 0.7 | 0.5 |

Table 3 shows nuclei (either 1.0-3.5 μm in diameter or <1.0 μm in diameter) versus aggregates (agg) in levitated droplets as a function of the induction potential, either 100 or 190 V, used to induce net charge during the formation of each droplet. The number of replicates performed with an induction potential of 100 V and 190 V was 12 and 6 respectively.

When solids were formed in these levitated droplets, only aggregates were observed in the droplets that were formed using an induction potential of 100 V (i.e. a relatively high mass-to-charge ratio for the reaction vessel), but in contrast, only nuclei were observed in the droplets that were formed using an induction potential of 190 V (i.e. a relatively low mass-to-charge ratio for the reaction vessel).

### 1.1.7 Use of Nuclei to Seed Formation of Crystals

### Experiment 6

A further experiment was performed to test the hypothesis that nuclei formed in a primary reaction vessel at low relative mass-to-charge could be delivered to a secondary nucleation vessel containing another solution to seed the formation of crystals.

The solution used for droplet generation and subsequent levitation of droplets with net charge was comprised of: i) 60 μL of a solution containing 20% glycerol to distilled deionized water by volume, ii) 40 μL of acetone, iii) 40 μL of a solution saturated in a-cyano-4-hydroxycinnamic acid (CHCA) in 50:50 v:v acetonitrile:0.1% TFA in distilled deionized water, iv) 180 μL of acetonitrile, and v) 80 μL of distilled deionized water. The solution pipetted directly onto the glass slide consisted of a saturated matrix solution of CHCA in 1/1:v/v: ACN/0.1% TFA in distilled deionised H₂O). The volume of this solution pipetted in each case was 50 μL.

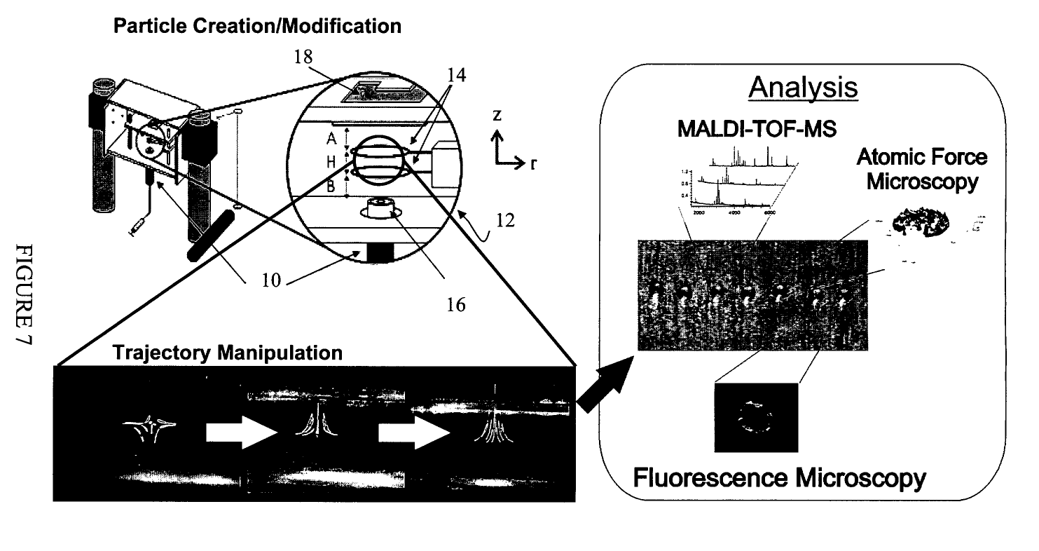

To provide a visual reference for CHCA, the solids formed from a solution simply deposited onto a glass cover slip are shown in FIG. 6A-6C. CHCA aggregates versus the nuclei are differentiated in these pictures because the former appear globular and dark whereas the latter appear sharp edged and lighter in color. FIG. 6A shows some of the largest CHCA crystals formed when a 10 μL aliquot of the starting solution was pipetted onto a glass cover slip and allowed to air dry. FIG. 6B is a picture that is representative of the crystals typically observed when an aliquot of the starting solution is pipetted onto a glass cover slip and allowed to dry. Likewise, the picture identified as 6C was taken after a similar aliquot of a starting solution was delivered by pipette onto a glass cover slip that was tilted to cause the solution to spread as a thinner film over a larger surface area of the glass cover slip.

FIG. 6D was taken after yet another aliquot of the starting solution was pipetted onto a glass cover slip, but this time, that aliquot of solution was immediately seeded by the deposition of 3 droplets that had been levitated in an EDB. CHCA nuclei were likely present in one or more of those three droplets because they had been created using a relatively high induction potential (200 V). Large CHCA crystals are apparent in FIG. 6D as a result of this seeding by levitated droplets containing CHCA nuclei.

This experiment can be classified as a two-step crystal design experiment, wherein the “primary reaction vessel” could be optimized for the formation of small nuclei of a dissolved solid (i.e. tuning of the chemical and physical description of a levitated droplet), and the “secondary nucleation vessel” that is seeded with nuclei could be optimized for crystal growth.

### Example 2.0

### 2.1 Measurement of Droplet Mass and Net Charge

The capability to process compounds in levitated droplets with net charge, such as promoting or catalyzing the nucleation of dissolved solids contained within them was demonstrated in Example 1.0. In this Example the inventors describe how droplets with net charge levitated in an EDB 12 can be filtered according to the mass-to-charge ratio of the droplets. This section also delineates measurements of the actual mass and net charge in individual droplets.

As described above, and shown in FIG. 1, wall-less sample preparation (WaSP) methodology is built around an electrodynamic balance (EDB), a device that uses AC and DC potentials applied to electrodes to trap charged droplets/particles at atmospheric pressure. Here, the ability of WaSP to preferentially eject specific particles was investigated with a focus on the effect of the mass-to-charge ratio (m/z) of the charged particles. Charged glycerol-based droplet residues on the order of 10¹¹ to 10¹² amu carrying 6×10⁵ to 6×10⁶ net excess charges (polarity: + or −), or 2×10⁶ to 2×10⁷ amu/e, were studied using the current electrode setup. Charged droplets of varied sizes were created and their net excess charge and masses measured using an electrometer and a radiochemical method based on liquid scintillation, respectively. Using these standardized charged glycerol droplets, a series of experiments were performed on pairs of droplets of differing m/z trapped in an EDB that showed droplets were controllably ejected from the EDB in order of highest to lowest m/z. This capability will be useful in studies investigating the chemistry of droplets/particles based on their m/z.

### 2.1.1 Experimental Apparatus and General Procedure for Measurement of Droplet Mass and Net Charge

All charged droplets studied in this Example were prepared using a piezoelectric droplet-on-demand dispenser 10 as described in Section 1.1.1 above fitted with a 60 μm diameter orifice. Methanol or aqueous solutions containing 0.8 to 8.0% glycerol were loaded into the ˜2 μL internal reservoir of the droplet dispenser using a 10 μL automatic pipette. Droplet formation and charging characteristics are described in detail below. The charge carried by single droplets was measured by delivering the droplets to a stainless steel plate connected to an electrometer (6517a, Keithley Instruments). A Faraday cage was required to reduce the background level so that the small charges (femtoCoulomb) carried by each of the droplets could be measured.

The EDB 12 used for wall-less sample preparation (WaSP) has been described in detail earlier in Section 1.1.1. In this Example the EDB 12 consisted of two copper wire (0.9 mm diameter) rings 14 (2 cm diameter) mounted parallel at a separation distance of 6 mm.[8] The amplitude of the 60 Hz AC potential applied to the ring electrodes (ACₜᵣₐₚ), in phase, ranged from 500 to 2,700 V_{0-P}. The vertical positions of droplets in the EDB 12 were manipulated by the DC potentials applied to the induction electrode 16 and the MALDI plate 18. Droplets levitated in the EDB 12 were illuminated via forward scattering by a 4 mW green HeNe laser (Uniphase model 1676, Manteca, Calif.). Images of levitated droplets were collected by focusing a digital camera through a microscope objective to the center of the ring electrodes. Note that to minimize the disturbance of the trajectories of levitated droplets, the droplet generator 10, the ring electrodes 14 of the EDB 12, and the target plate 18 were enclosed in a plexiglass chamber to eliminate excessive air flow (FIGS. 1 and 7).

Single droplets were dispensed directly into liquid scintillation vials using time dependent waveforms applied to the piezoceramic droplet dispenser 10. 2 mL of scintillation cocktail (Amersham Biosciences) was added to the scintillation vial that was then vortexed for 30 seconds. Scintillation is the name given to the detection of fluorescence emission from a compound that was itself excited into an electronic state above the ground state by the absorption of a thermalized electron and the thermalized electron was the result of quenching a hot electron generated by a nuclear decay event. The detection of the fluorescence emission originating in the scintillation vial enables quantitative measurement of the total number of radionuclides in the scintillation vial.

All activity measurements from single droplets and stock solutions were performed using a liquid scintillation counter (LKB Wallac 1217 RackBeta, Fisher Scientific, Montreal, Quebec). Each measurement had an integration time of 10 minutes. Working with ³²P requires many safety precautions including the use of plexiglass shielding, a Geiger-Mueller counter, and swipe tests in the working area to monitor for possible spills.

In order to demonstrate the charged droplet filtering capabilities of WaSP, the inventors first established a reproducible method for creating standard charged droplets of known m/z. The basic method is set forth in Example 1 above.

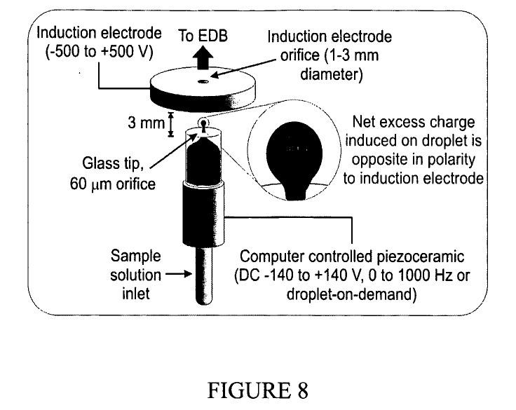

As described above, droplets composed of a low volatility solute such as glycerol have long been the vehicle of choice for EDB applications. To create the glycerol droplets, a solution of 3% glycerol in methanol was loaded into the droplet dispenser's internal reservoir (FIGS. 2 and 7). After applying a potential to the piezoceramic (10-75 V), a droplet was dispensed because the cylindrical piezoceramic reduced its dimensions, constricted the glass sample reservoir and ejected a droplet from the 60 μm orifice at its tip. As explained above, in order for the dispensed droplets to be levitated in an EDB 12 they must carry net excess charge. In accordance with the inventors' levitation procedure described above, an electrode (induction) was positioned 3 mm from the orifice of the droplet dispenser (FIG. 8) and by varying the DC potential applied to it (IP_{f}, the induction potential during droplet formation), the net excess charge carried by the droplets was controlled. The induction electrode had a 2 mm orifice positioned directly in line with the droplet dispenser's orifice so that the droplet was delivered through it towards the EDB 12.

### 2.1.2 Measurement of Net Excess Charge

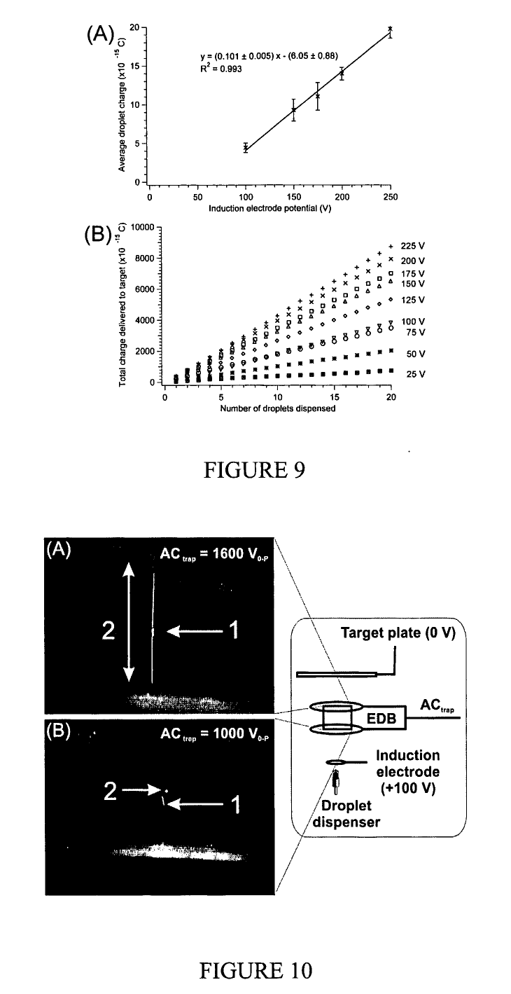

To measure the net excess charge induced into the single dispensed droplets by the induction electrode 16, each droplet was captured on a stainless steel plate. Upon impact of the droplet, the charge delivered to the plate was measured using an electrometer with femtocoulomb (fC) sensitivity. The charge on single droplets prepared with ±10 V_{0-P} (the minimum required to create a droplet) applied to the piezoceramic in the droplet dispenser delivered too little charge to be measured one at a time so 100 droplets were dispensed at 100 Hz. The total charge delivered was measured and divided by 100 to calculate the average charge carried by single droplets. This experiment was repeated 20 times (2000 droplets) for four different IP_{f}. FIG. 9A shows that the average droplet charge increased linearly as IP_{f} was increased from 100 to 250 V. The charge carried by the droplets was negative because a positive IP_{f} was used.

To investigate the reproducibility of single droplet charging, the amplitude applied to the piezoceramic in the droplet dispenser was increased (30 V_{0-P}) to create larger droplets. This enabled the measurement of the charge carried by single droplets because the droplets were bigger (see mass measurements below) and thus carried more charge. At a fixed IP_{f}, twenty droplets dispensed at 0.5 Hz and the total charge delivered to the plate was measured after each single droplet impacted with the plate. By repeating this experiment for IP_{f} set at 9 different values (25, 50, 75, 100, 125, 150, 175, 200, 225 V), droplet charging was deemed reproducible by the linearity of the plot of the total charge delivered as a function of the number of droplets dispensed (FIG. 9B, note scale difference relative to FIG. 9A). Overall, the data in FIG. 9, and similar experiments performed by the inventors show that the charge carried by the droplets was controllable by varying IP_{f} and the values varied between 5 to 1000 fC per droplet. This corresponds to 3.1×10⁴ to 6.3×10⁶ net excess elementary charges per droplet.

### 2.1.3 Measurement of Mass of Droplets

To calculate the m/z of the droplets created, it is necessary to know the mass of the droplets. A radiochemical method was used to measure the volume of droplets as a function of amplitude applied to the piezoceramic in the droplet dispenser, allowing us to calculate the mass of the droplets. A 100 μL solution of 3.7 MBq ³²P labeled orthophosphate in a mixture containing 89.2% water, 10% methanol, and 0.8% glycerol was prepared. By depositing single droplets into liquid scintillation vials and comparing the number of Becquerels measured from them relative to the number measured from a 1.000±0.005 μL aliquot of the ³²P in glycerol stock solution, we could determine the volume of the single droplet delivered.

The average activity measured from the single droplets was 0.026±0.001% that measured from the 1.000 μL sample so the average initial droplet volume was 2.6×10⁴±1×10⁻⁵ μL or 260±10 μL. The solution loaded into the droplet dispenser to create the droplets contained 0.8% glycerol by volume so the initial droplets dispensed contained 2 μL of glycerol. When the droplets are levitated in the EDB 12, volatile solvents rapidly evaporate to leave behind a droplet residue composed of essentially 100% glycerol. (Corrections to account for the water in the droplet that does vary with the relative humidity of the air in the levitation chamber were made subsequently to these calculations). Therefore, the final droplet volume was ˜2 μL. By assuming that the droplet was 100% glycerol and using the density of glycerol (1.259 g/ml), the mass of the levitated droplets on average was calculated to be 2.5×10⁻⁹ g. Converting this to atomic mass units (u), the droplets were 1.5×10¹⁵ u. Recalling that the glycerol droplets carried charges ranging from 5 to 1000 fC, the m/z of the levitated droplets ranged from 9.6×10⁸ to 2.0×10¹¹ u/e.

Droplets of many different compositions can be prepared using WaSP so it is useful to look at the effect of composition on their m/z. If the percent glycerol in the starting solution was changed to 8%, the final levitated droplet residue would have a volume of ˜21 μL, thereby increasing the mass (and m/z) by 20 times. The inventors have successfully levitated droplets created from solutions containing a percent glycerol by volume of 0.8 to 10%, creating final levitated droplets of 1×10¹⁵ to 20×10¹⁵ u. The inventors have also shown that the charge carried by the droplets can also be varied from 10⁴ to 10⁶ e. By combining the control of the mass and charge of the glycerol droplets, a wide variety of possible experimental scenarios has been established. Note that these values are many orders of magnitude greater than the typical m/z of ions measured in a quadrupole ion trap mass spectrometer. This characteristic will prove valuable in creating unique applications for this technology in the future.

### 2.1.4 Filtering of Droplets Based on m/z Ratios

Using the standardized charged glycerol droplets described above, experiments designed to elucidate whether WaSP could be used to filter the droplets based on their m/z were performed. The general experimental approach was to trap two droplets simultaneously, one created with high m/z and one low m/z relative to each other. By measuring the DC potential applied to the target plate used to eject each droplet, the filtering capabilities of WaSP could be evaluated. An important factor in this experimentation was differentiating between the high and low m/z droplets once trapped. To do this, both high m/z (IPf=50 V) and low m/z (IPf=100 V) droplets were levitated alone (ACₜᵣₐₚ=1600 V_{0-P}) and their respective trajectories were compared. Once trapped, the low m/z droplet immediately adopted a trajectory elongated along the z-axis whereas the high m/z droplet was more focused towards the null point (the center of the EDB). When levitated simultaneously, the high and low m/z droplets adopted trajectories similar to when they were levitated alone so this property was used to identify them (FIG. 10A). The images in FIG. 10 were collected using a digital camera focused through a microscope. What was actually photographed was the forward scattering of the green light from the HeNe laser used to illuminate the droplets. When imaged, the droplets did not look like single points because they oscillated at 60 Hz, the frequency of ACₜᵣₐₚ applied to the ring electrodes (observed at the top and bottom of the images in FIG. 10). The shutter speed on the digital camera was not fast enough to capture the droplets in a single spot so the average integrated trajectory of many oscillations of the droplet was captured. This same principle applies to the images in FIG. 10 demonstrating the trajectory manipulations of the population of levitated droplets.

To levitate both high and low m/z droplets simultaneously, ACₜᵣₐₚ was set to 1600 V_{0-P}, a high m/z droplet was levitated, and then a low m/z droplet was injected in to the EDB. Attempts to reverse the order of injection resulted in failure to capture the high m/z droplet. When ACₜᵣₐₚ was decreased to 1000 V_{0-P}, the trajectory of the low m/z droplet became focused towards the null point, whereas the trajectory of the high m/z droplet moved closer to the induction electrode (FIG. 10B). Next, the DC potential applied to the induction electrode was changed to 100 V. The target plate potential was slowly increased until a single droplet was ejected from the EDB and deposited on the target plate (this value was designated the deposition potential, DP). In all iterations of the experiment the high m/z droplet was deposited first. The lower m/z droplet was deposited when the potential applied to the target plate was further increased. This procedure was repeated for droplets created at IP_{f}=50 V and IP_{f}=150 V, although in this variation of the experiment a larger ACₜᵣₐₚ (2150 V_{0-P}) was required to trap the two droplets.

Table 4 below summarizes the DP measured for each droplet in a series of droplet pairs whose relative m/z varied. The DC potential applied to the MALDI plate required to eject each droplet of a pair of droplets created with varied induction electrode potentials is shown.

TABLE 4

| Induction potential applied | | | | | |
|---|---|---|---|---|---|
| during droplet formation | | | Potential applied to | | |
| (IP_{f}), volts | | | target plate (DP), volts | | |
| | 1^{st} | 2^{nd} | 1^{st} | 2^{nd} | ΔDP |
| Identical m/z | 50^{a} | 50^{a} | 58.6 ± 0.5 | 58.9 ± 0.3 | 0.3 ± 0.6 |
| pairs | 100^{a} | 100^{a} | 66.8 ± 1.9 | 69.1 ± 3.5 | 2.3 ± 4.0 |
| | 150^{b} | 150^{b} | 91.7 ± 3.0 | 95.8 ± 4.7 | 4.1 ± 4.9 |
| | 50^{b} | 50^{b} | 75.2 ± 0.9 | 76.3 ± 1.7 | 1.1 ± 2.3 |
| Different m/z | 50^{a} | 100^{a} | 61.0 ± 1.0 | 72.0 ± 1.7 | 11 ± 2.0 |
| pairs | 50^{b} | 150^{b} | 75.4 ± 1.7 | 99.6 ± 0.7 | 24.2 ± 2.2 |

ACₜᵣₐₚ = 1600 V_{0-P}., (b) ACₜᵣₐₚ = 2150 V_{0-P}.

Each value in Table 4 is the average of the values measured for five separate pairs of droplets and the errors indicate the magnitude of one standard deviation. Pairs of droplets created at identical IP_{f} (identical m/z) were ejected from the EDB at the same DP, within experimental error. However, the second droplet deposited always required a slightly higher average DP. With droplets created at different IP_{f} (different m/z), the droplet with the highest m/z was always deposited first. A higher ACₜᵣₐₚ was required to trap the lower m/z droplets so the DP of a droplet that was created with IP_{f}=50 V and trapped with the droplet created with IP_{f}=100 V was lower than when it was trapped with a droplet created with IP_{f}=150 V. This was a direct example of the effect of ACₜᵣₐₚ on DP we have observed in the past. Overall, the preferential ejection of the highest m/z droplet upon increase of the target plate potential indicated that m/z was a factor in ejection order and hence the EDB essentially acted as a mass filter for the charged droplets.

As should be apparent from this Example, there are two ways that WaSP could be used to filter droplets. The first would be to trap a population of droplets, anywhere from 1-50 droplets, in the EDB. Then, single droplets can be ejected from the balance onto a target based on their m/z as described above, while the remaining droplets remain trapped. If desired, this stably levitated population could then be exposed to gas-phase reactants to modify their chemistry before their ejection, enabling experimental determination of their environment on the ability to identify them. The second approach would be to set up the EDB to act as a bandpass filter, allowing only a certain range of m/z to pass through it, automatically ejecting the droplets in less than 500 ms. This method would be useful for rapidly sorting of droplets based on their m/z and could be readily automated.

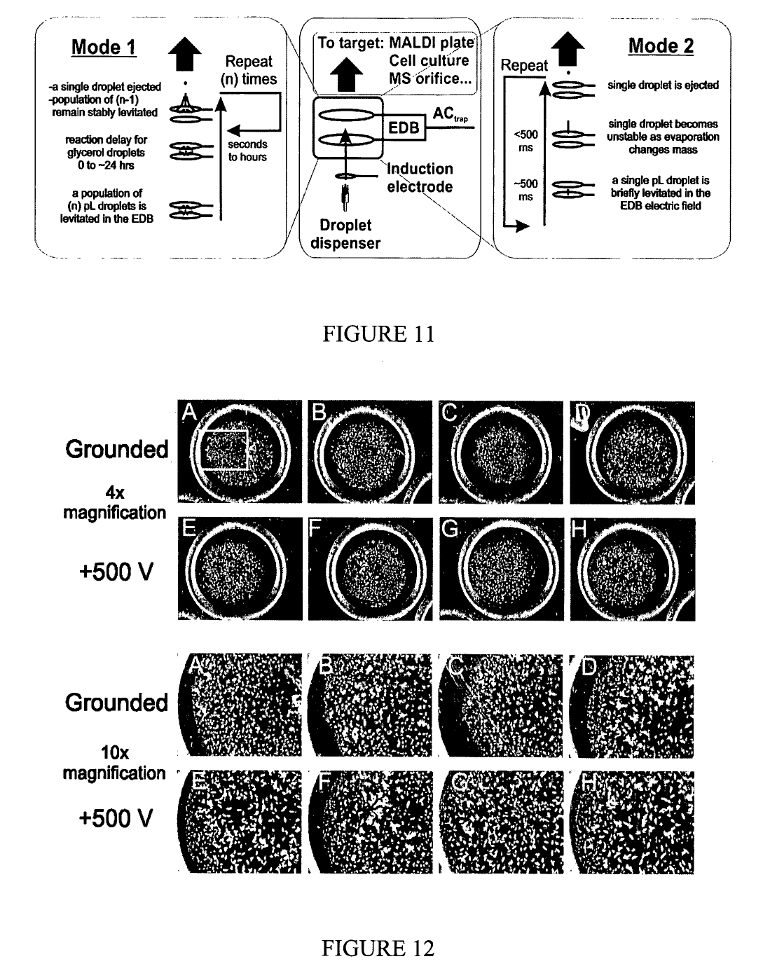

During the development of these modes of operation the inventors used a translatable collection plate to make the demonstration clearer. In the first mode, the delayed ejection mode (FIG. 11, mode 1), one droplet at a time was ejected from a population of droplets trapped in the EDB by using an attractive potential applied to the target plate. The position of the target plate relative to the EDB was changed in between each droplet ejection event, thereby creating an array of deposited droplets. Alternatively, a rapid ejection mode was developed (FIG. 11, mode 2). Single droplets were created (IP_{f}=20 V) at a frequency of 1 Hz while ACₜᵣₐₚ=2700 V with the potential applied to the collection plate set at 200 V. This caused each droplet to be briefly trapped in the EDB, allowing the methanol to evaporate. In <1 s each droplet escaped the electric field of the EDB and was ejected along the z axis at x=y=0. By moving the collection plate between each droplet generation event, an array of deposited droplets was formed. The accuracy of droplet deposition using both methods, partially limited by the manual micrometer translation of the collection plate, has been measured by comparing the final position of the deposited droplets with their expected positions, yielding an average variation of ±5 μm.[124] The flexibility of this basic procedure could be significantly expanded by introducing computerized control of the collection plate translation and the potentials applied to the EDB electrodes, all synchronized to the droplet generation event.

There are several aspects of WaSP charged droplet filtering that make it very flexible with respect to the types of application for which it could be used. First of all, the droplets that are filtered by WaSP are created from a starting solution of choice, therefore putting very little limitation on the potential analytes studied, such as single bacteria or synthetic inorganic particles. Next, the m/z of the droplets does not have to fit within the range that was demonstrated in this Example. By changing the characteristics of the electric field, WaSP can be easily modified to filter alternate m/z's. The source of the charged particles is also not limited to strictly charged droplet dispensers. The WaSP approach is potentially applicable to filter atmospheric particles using alternate charging mechanisms. Lastly, the target for particle collection is not limited to a stainless steel plate. The inventors have also delivered particles onto populations of cells on a glass slide and into the orifice of a mass spectrometer.

Overall, the flexibility of this charged particle filtering methodology based on WaSP has proven valuable in the development of such applications as the preparation of μm-sized sample spots for MALDI-TOF-MS and the study of multi-phase heterogeneous reactions on single aerosol particles. Some other potential applications of an using an EDB as a particle filter include: (1) a delivery module for a bioaerosol mass spectrometer attempting to detect single cells, bacteria, or viruses, (2) isolation of inorganic particles for subsequent studies based on their m/z, (3) an aerosol particle sorting mechanism, (4) a tool for performing studies of aerosol particle reactivity or nucleation capabilities based on their m/z, or (5) the single particle dosing of cell populations for medical research.

### 2.1.5 The Effect of a Charged MALDI Plate on CHCA Crystallization and Ion Production

The rationale for performing the experiments described in this section was to confirm that the potential applied to the plate being used as the target for deposition of a levitated droplet/particle was not influencing test results.

In the above Examples, the nucleation of CHCA was described as being influenced by the mass-to-charge of the reaction vessel (e.g. in a droplet). However, in all of those experiments, the extent of nucleation was not measured in situ (i.e. in the levitated droplet) but rather only after the levitated droplet had been deposited on a target. The target was either a stainless steel plate to which a potential was applied to attract the levitated droplet or a glass cover slip positioned on top of the stainless steel plate to which a potential was applied. To eliminate the possibility that the deposition of the droplet from the EDB onto a substrate was responsible for the observations made above, the inventors proceeded to pipette an aliquot of a stock solution onto a biased stainless steel plate. Note that in these experiments, even though the plate is charged, the droplet deposited onto it remained net neutral because within the droplet a double layer would be established immediately upon contact with the plate. So, even though the actual material deposited onto a plate in this section was a neutral droplet, and thus different from the deposition of a droplet with net charge, the effect of the deposition onto a biased plate on the nucleation of dissolved solids was the objective of this experiment. In other words, the hypothesis was, does the drying of a droplet on a biased plate affect crystal formation and growth, and does that in turn affect signal-to-noise ratio (S/N) of the analytes contained in the droplet?

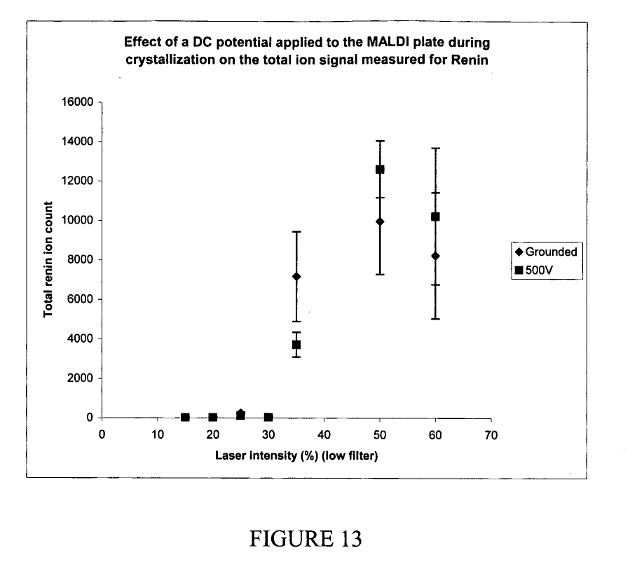

A stock solution of 10 pmol/μl of renin was prepared in distilled deionized water with 0.1% trifluoroacetic acid. The MALDI matrix α-cyano-4-hydroxycinnamic acid (CHCA) was prepared at 10 mg/ml in 50:50 methanol:acetic acid. 10 μl of each solution was mixed in a microcentrifuge tube and vortexed. The MALDI plate was connected to a DC power supply of +500 V. Four 1 μl aliquots were deposited on to the MALDI plate as four discrete sample spots and allowed to air dry for 15 minutes. The MALDI plate was then grounded and four more 1 μl aliquots were deposited onto the MALDI plate and allowed to air dry for 15 minutes. Images of the sample spots were collected through an optical microscope at 4 and 10 times magnification (FIG. 12). Mass spectra were then collected on a MALDI-TOF-MS in reflectron mode (M@LDI-LR, Waters Technologies Inc., Manchester, U.K.). The mass spectrometer was programmed to collect 10 mass spectra, comprised by the average of 10 laser shots, at 10 random positions within each sample spot at a fixed laser intensity. This procedure was repeated for five different laser intensities ranging from below to above the threshold required for ionization of the renin, the results of which are presented in FIG. 13.

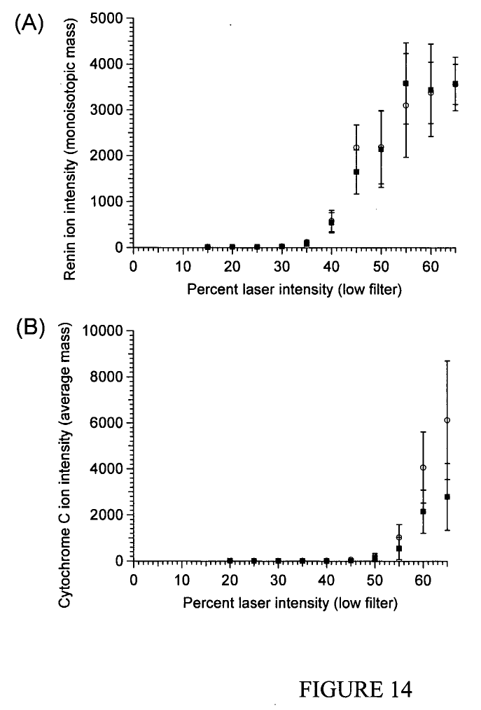

The plate onto which the sample was aliquotted using a micropipette was biased to 1000 V, and the experiment repeated. The analysis of the data acquired using MALDI-TOF-MS of the sample materials prepared on a grounded plate and a plate biased to 1000 V during the period that the droplets dried are presented in FIG. 14.

Examination of the data presented in FIGS. 12-14 indicate that the potential of the plate on which a droplet dries does affect the appearance of the crystals that result. This observation could be of use for crystal growth. However, the S/N of the analyte (target solute) in the droplets is not significantly different from that obtained when a droplet is dried on a grounded plate.

### Example 3.0

### 3.1 MALDI Matrix and Analyte Compound Co-Precipitates

This Example illustrates of how the invention could be applied to the growth of crystals of a MALDI matrix within which an analyte compound co-precipitates. Prior work from other groups has indicated that homogeneous co-crystallization of a solute with a matrix compound is an important factor for obtaining good signal-to-noise (S/N) ratio for the analyte when characterized using MALDI-TOF-MS.[126] Furthermore, the quality of the crystals of a matrix that form on a surface has been characterized as heterogeneous.[127-128] This body of information suggests that there is a need for methodology that enables the formation of good quality crystals within which there is an analyte distributed homogeneously within that crystal in order to realize adequate analytical performance, such as analyte S/N, when characterized by MALDI-TOF-MS.

Several sets of experiments were performed in which droplets were created using different induction potentials. Each experiment was performed with a starting solution prepared daily with the following composition: 40 μL Acetone, 100 μL of a CHCA solution that was originally prepared by mixing 1:1 v:v acetonitrile:0.1% TFA in distilled deionized H₂O, 60 μL of water/glycerol solution that was originally prepared by mixing 1:4 glycerol:distilled deionized H₂O, 50 μL of ACTH solution with a concentration of 10 μM which makes the final concentration in the solution used for levitation 1 μM, and 150 μL Acetonitrile (ACTH=Adrenocorticotropic Hormone, Fragment 18-39). All droplets were levitated for a period of 5 minutes prior to their deposition onto a substrate remote from the EDB. The substrate was a stainless steel MALDI plate in this example.

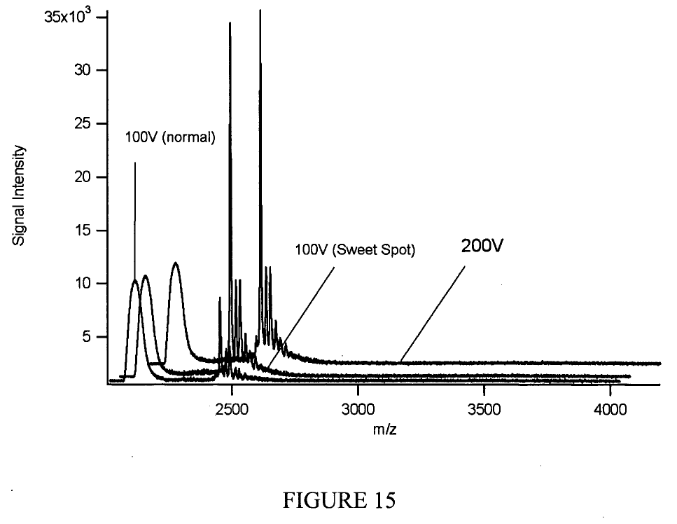

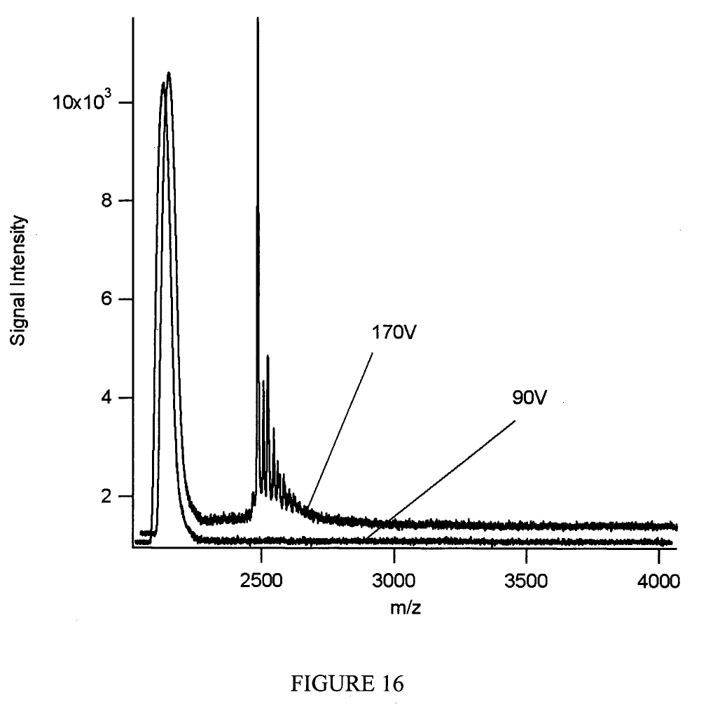

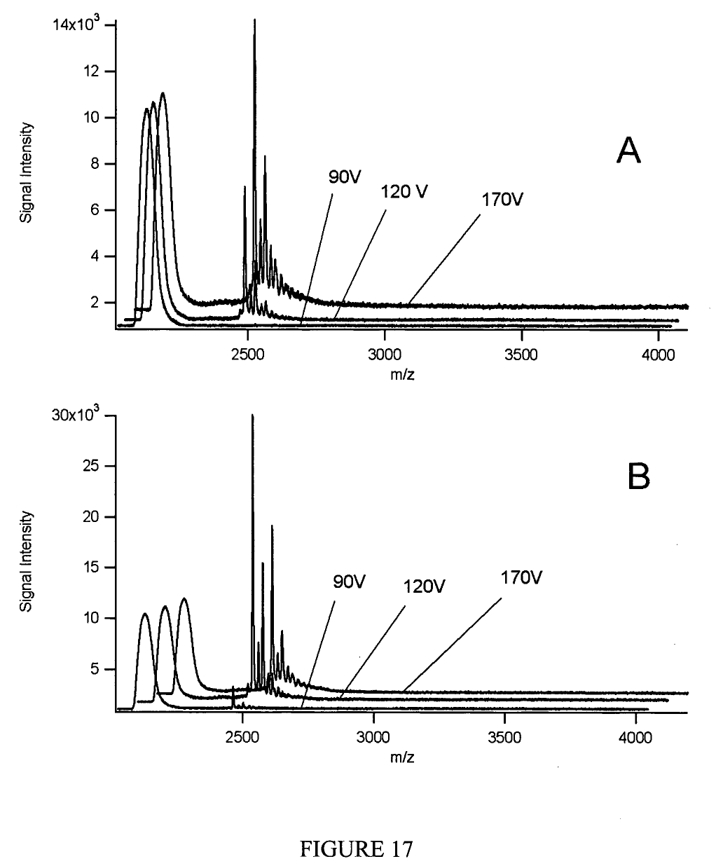

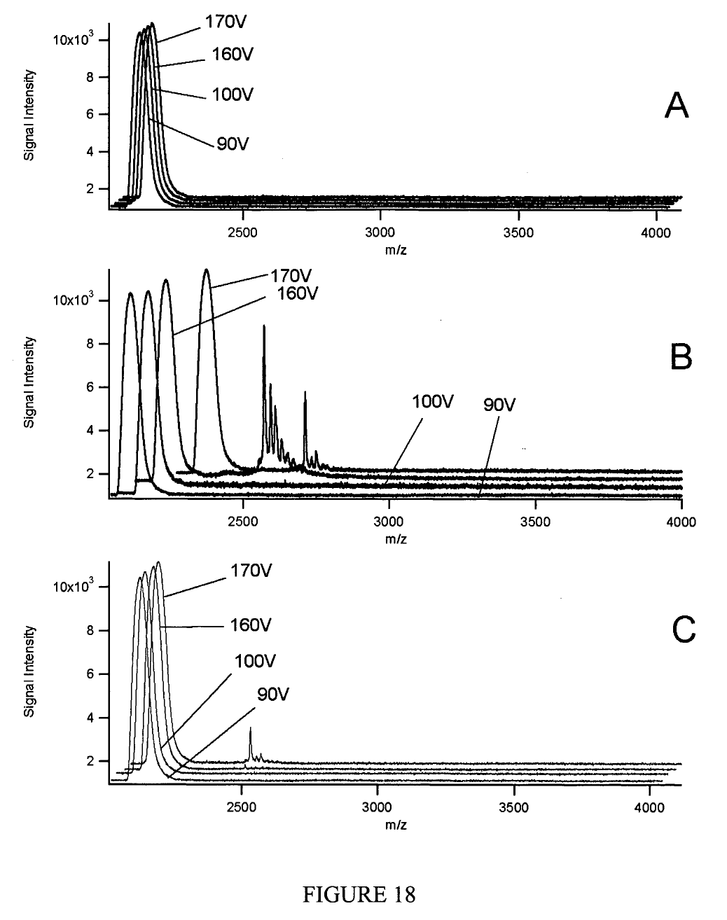

The MALDI plate was then inserted into MALDI-TOF-MS (model Voyager, Perceptive Biosystems, MA) and the levitated droplet residues targeted by the laser and the mass spectra obtained are displayed in FIG. 15-18.

Based on the data presented in these figures, the utility of a high induction potential during the droplet formation results in an improved S/N ratio for an analyte compound in the starting solution.

### Example 4.0

### 4.1 Promotion of CHCA and Peptide Cocrystallization Within Levitated Droplets Having Net Charge

### 4.1.1 Experimental Procedures

Procedures for dispensing droplets with net charge, measurement of initial droplet net charge and volume, droplet levitation, and matrix-assisted desorption/ionization mass spectrometry are described above. In this experiment promotion of MALDI matrix/peptide cocrystallization in levitated droplet residues was demonstrated.

Two starting solutions were used in the cocrystallization studies. A single component peptide solution was prepared by mixing 40 μL of acetone, 100 μL of a saturated solution of CHCA in 50:50 acetonitrile (ACN):0.1% trifluoroacetic acid (TFA) in H₂O, 60 μL 20% glycerol in H₂O, and 50 μL of 10 μM adrenocorticotropic hormone fragment 18-39 (ACTH) in H₂O (0.1% TFA). A multi-component peptide solution composed of 40 fmol/μL angiotension II and bradykinin, 4 fmol/μL angiotension 1, 0.2 mg/ml CHCA, 20% methanol, 20% ACN, 1.5% glycerol, and 0.6% TFA in H₂O was also prepared. An aliquot of the starting solutions was loaded into the approximate 5 μL reservoir of the droplet dispenser using a 10.00 μL automatic pipette.

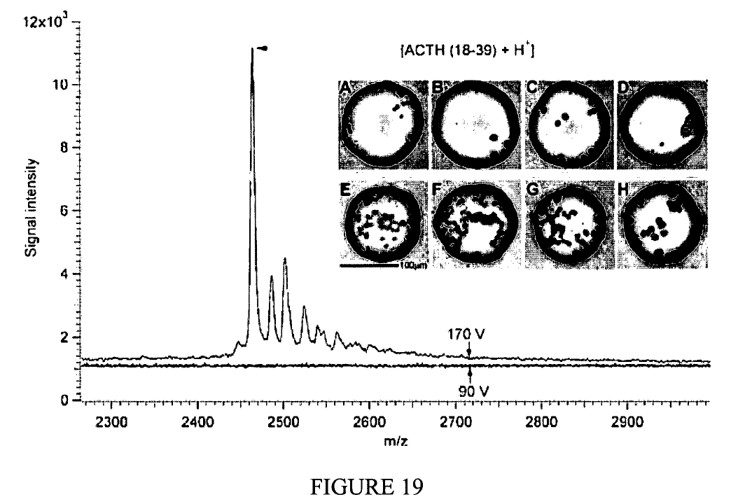

The single component peptide starting solution was used to generate 10 droplets that were each levitated for 2 min prior to depositing all of them at a single location on a glass slide to create a single sample spot. Optical microscopy of the sample spot composed of 10 droplet residues showed that CHCA precipitates were created during levitation. Insets a-d of FIG. 19 show four examples of sample spots formed from a population of 10 droplets created with the induction electrode potential set at 90 V (referred to hereafter as IP_{f,90V} droplets). MALDI-MS spectra were acquired at a fixed laser irradiation setting from sample spots prepared by codepositing the residues of 10 IP_{f,90V} droplets onto a MALDI target plate, yet no ion signals were detected that could be attributed to ACTH or a fragment thereof (FIG. 19). The experiment was performed again with IP_{f,170V} droplets. These sample spots contained substantially more CHCA precipitates (FIG. 19, inset e-h) than the residues of the IP_{f,90V} droplets (FIG. 19, inset a-d). Ion signals for the intact peptide ACTH [ACTH⁺H⁺], m/z=2465.2, were detected by MALDI-MS analysis of sample spots created from the residues of 10 codeposited IP_{f,170V} droplets (FIG. 19). The ion signals detected in the region of m/z=2480-2730 were ACTH containing clusters with cations Na⁺, K⁺, and solvent molecules. The same laser irradiation energy was used throughout in acquiring the spectra plotted in FIG. 19. Precipitate abundance as observed by optical micropscopy, and ion count as detected by MALDI-TOF-MS were similar in sample spots created from levitated droplets with the polarity of the net charge reversed.

The data presented in FIG. 19 indicated that the abundance of precipitates formed during the period of time the residues remained levitated in the EDB was dependent on the magnitude of the net charge imparted onto the original droplets. High ACTH ion abundance from the IP_{f,170 V} droplet residues reflected the increased amount of ACTH cocrystallized with CHCA relative to the precipitates in the IP_{f,90 V} droplet residues. These results were consistent over a series of 20 experiments by two investigators using different solutions, droplet dispensers, and experimental apparatus. Although the increased ion abundance can be explained by the increased MALDI matrix/peptide crystals observed in the residues of droplets with high net charge, the question of why this differential chemical processing was occurring was not answered. Experimental methodology designed to delineate the source of this unexpected phenomenon and to better characterize the properties of the initial dispensed droplets, their residues that were levitated in the EDB, and the precipitates that formed in those residues while levitated are described in the remainder of this Example.

### 4.1.2 Levitation and Ejection of Different m/z Droplets

Inducing varied net charge on dispensed droplets of constant volume changed their respective m/z. To effectively study droplets of differing m/z using the EDB, it was important to delineate whether droplets of different m/z could be differentiated from each other while levitated in the EDB. First, an individual IP_{f,150V} droplet was levitated. After noting its trajectory, it was ejected and then a single IP_{f,150V} droplet was levitated at the same ACₜᵣₐₚ. Different trajectories for these two droplets were discernible to the naked eye. Next, an IP_{f,50V} and an IP_{f,150V} droplet were levitated simultaneously in the EDB. Again, their trajectories were clearly different. This observation enabled repeat experiments to be performed on two different m/z droplets simultaneously levitated in the EDB because it was possible to visually track which droplet was which.

Another way to differentiate between the two droplets of different m/z was developed when the DC potential applied to the target required to eject each droplet was measured. For all repetitions of the experiment, the IP_{f,50V} droplet was ejected from the EDB first and the IP_{f,150 V} droplet remained levitated. This observation was consistent with all combinations of two different m/z droplets levitated in the EDB (i.e., IP_{f,150V} droplet ejected before IP_{f,150V}). With only two droplets that had the same m/z levitated, such as two IP_{f,50V}, IP_{f,100V}, or IP_{f,150V} droplets, the trajectories of the droplets were similar and their deposition potential (DP) values were identical within experimental error [124]. Thus, two separate and complementary procedures were developed to differentiate between two different m/z droplets levitated in the EDB, facilitating further experiments designed to characterize differential chemical processing as a function of droplet net charge.

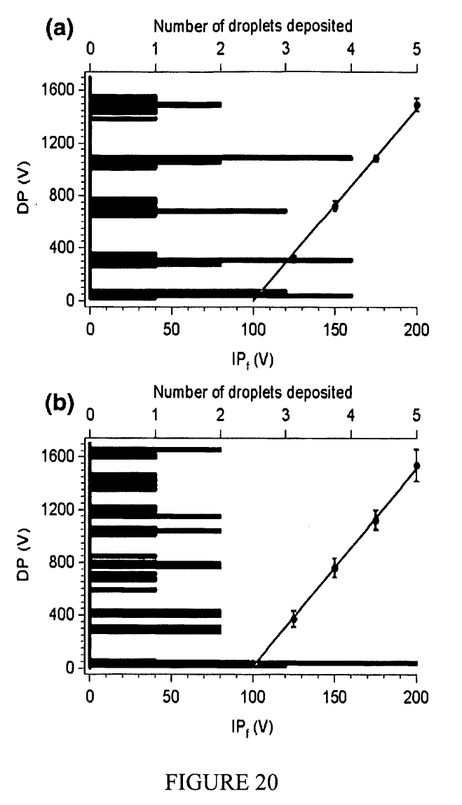

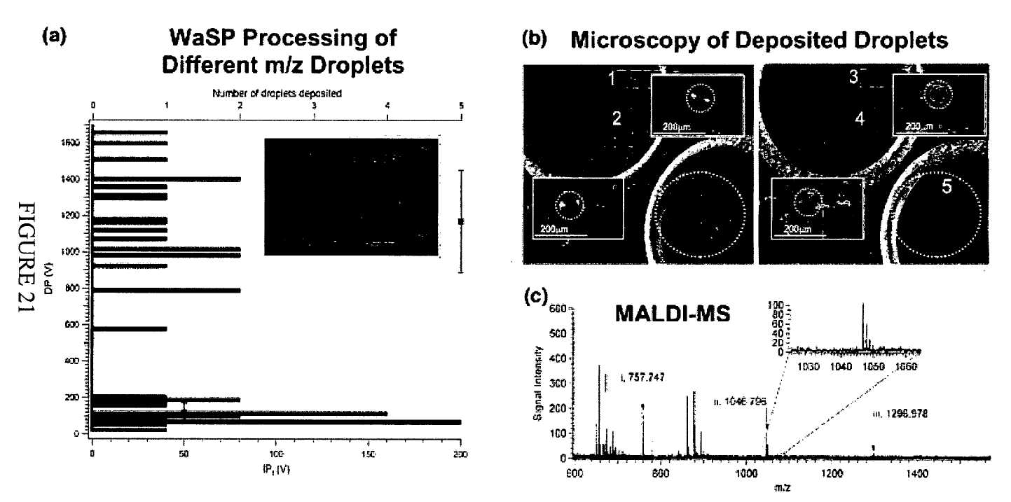

Next, the complexity of the experiment was increased. A population of five droplets was injected into the EDB where each had been created at IP_{f}=100, 125, 150, 175, and 200 V, respectively. The DC potential applied to the induction electrode was adjusted to 0 V after 2 min of levitation time. The voltage applied to the target was then manually ramped to higher values and DP was measured for each of the five levitated droplets. This procedure was repeated for nine more sets of five droplets. A plot of mean DP versus IP_{f} was linear over the range IP_{f}=100 to 200 V, indicating that none of these droplets underwent a Coulomb explosion (FIG. 20a). The mass change between the initial droplet and its levitated residue was therefore able to be determined simply based on evaporation of the volatile solvents contained in the starting solution only.

The histogram shown on the left hand axis in FIG. 20a displays the number of droplets deposited versus the deposition potential. To build the histogram an arbitrary bin size of Δ15 V was used to count droplets that were deposited from the EDB. This presentation format of the data shows five clusters corresponding to the droplets that were created at each of the five IP_{f} values used, and therefore each cluster represents a collection of droplets of unique m/z. The droplets created with IP_{f}=100, 125, 150, 175, 200 V have values of m/z=2.2×10¹⁰, 1.7×10⁹, 1.3×10⁹, 1.1×10⁹, and 9.5×10⁸, respectively, at the time of ejection from the EDB. The histogram of different m/z droplets can be considered analogous to the time-based histogram of ion arrival at the detector of a TOF-MS in specified time periods that is then converted to a m/z axis by calibration using ions of known m/z. Therefore, the histogram represents the raw data for m/z filtering of levitated droplets, which can be construed as a mass spectrum. The resolution for ejection of levitated droplets from the EDB was evaluated as 6 using R_[m/z₁₅₀/(m/z₁₅₀-m/z₁₇₅)] where m/z₁₅₀ and m/z₁₇₅ represent the m/z values of the IP_{f,150V} and IP_{f,175V} droplets, respectively.

Next, the number of droplets in the EDB was increased to 10 by injecting two droplets created at each distinct IP_{f} (FIG. 20b). Droplets with m/z differences in excess of the resolution for ejection from the EDB were trapped simultaneously and deposited onto the substrate according to their m/z, in order from highest to lowest m/z. However, there were two distinct potentials for the deposition of the first versus the second droplet that had been created using identical conditions. The explanation for this effect is Coulomb repulsion between the two droplets that had m/z values that were within the experimental error of being the same, and as a result, both droplets adopted similar trajectories within the EDB. The result of their repulsive interaction is that the m/z resolution for droplet ejection degraded [124]. For comparison, Coulomb repulsion effects impact the resolution in a three-dimensional quadrupole ion trap mass spectrometry when there are ˜1×10⁶ ions trapped with helium bath gas at a total pressure of 1 mTorr [25-27], so Coulomb repulsion in our experiments was not unexpected because each of the levitated droplets carried a net charge of ˜1×10⁶ ions.

### 4.1.3 Methodology for the Study of Chemistry within Droplets with Varied Net Charge

Using an aliquot of the multi-component peptide starting solution to load the droplet generator, 20IP_{f,50V} droplets and 20 IP_{f,200V} droplets were created (and injected) sequentially into the EDB and those two populations levitated simultaneously (FIG. 21a, inset). After the last droplet was injected into the EDB, the potential of the induction electrode was reduced to 0 V. Following a levitation period of 2 min the 40 droplets were filtered according to their m/z upon their ejection from the EDB (FIG. 21a). Two spots of material were created during the deposition of droplets from the EDB. One spot was comprised of the first twenty droplets ejected from the EDB (IP_{f,50V} droplets), and the second spot was formed by depositing the remaining twenty levitated droplets (IP_{f,200V} droplets) onto a different location of the target. These two sample spots are identified as region 1 and 2 in the inset of FIG. 21b, and were comprised of the droplets of low (IP_{f,200V}) and high (IP_{f,50V}) m/z, respectively. Note that because the rate of droplet generation was 1 Hz, the IP_{f,50V} droplets were levitated longer than the IP_{f,200V} droplets. This difference in time to form precipitates was not a limiting factor because there were consistently more precipitates in the IP_{f,200V} droplets that were levitated for a shorter period of time. Attempts to perform this experiment in reverse order of droplet injection were unsuccessful because with IP_{f,200V} droplets levitated, the momentum imparted onto the IP_{f,50V} droplets during their formation, which facilitated their injection into the EDB, resulted in their passing through the EDB because of Coulomb repulsion from the IP_{f,200V} droplets.

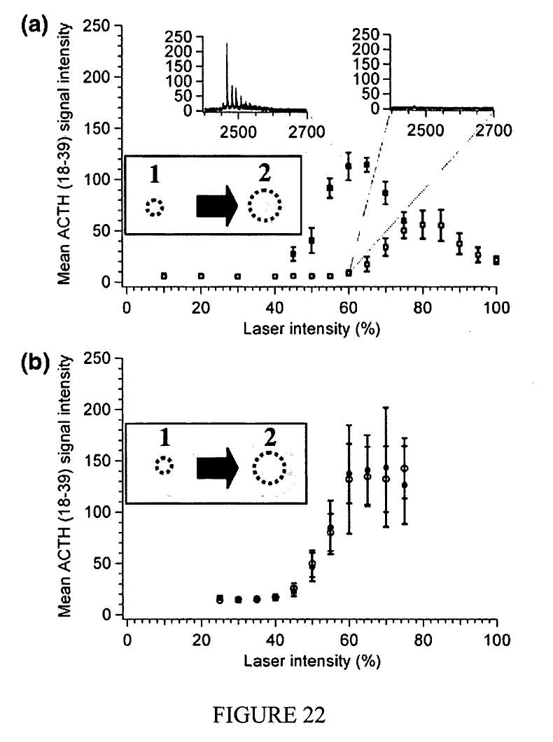

By inserting the MALDI plate into a vacuum chamber (˜3×10⁻⁷ torr), the glycerol from these spots evaporated, facilitating observation of the solids that had formed in the levitated droplets by light microscopy (inset 3 and 4, FIG. 5b). These two spots of sample were targeted with the N₂ laser set at an irradiation threshold of 76% (75 μJ/pulse). Signals corresponding to the three peptides (i: bradykinin, ii: angiotension II, iii: angiotension I) contained in the solution used to make the droplets were detected from the spot comprised of IP_{f,200V} droplets (net charge=290 fC) (FIG. 22c), but not from the IP_{f,50V} droplets (net charge =140 fC). The signal-to-noise ratio (S/N) of the ions detected from the 220 amol of angiotension II in the 81.5 μm diameter sample spot was 11 and only 0.0054 μl of sample was consumed. For comparison, a sample spot created by pipetting 0.25 μL of the multicomponent peptide starting solution (FIG. 5b, region 5, 10 fmol of angiotension II in the aliquot) which resulted in a spot diameter of 1390 μm diameter, yielded a S/N of 37 for angiotension II. Because the mass spectrum from the 0.25 μL sample spot was collected without changing the position of the laser spot (200 μm diameter), only ˜830 amol of angiotension II was irradiated. This accounts for the four times decrease in S/N observed from the 20 droplet sample spot and therefore essentially identical signals were obtained from that sample spot while consuming 50 times less sample solution.

Another replicate of the experiment was performed to prepare fresh sample spots. A sample spot was created from 12 codeposited IP_{f,90V} droplets and another sample spot was created from 12 codeposited IP_{f,170V} droplets. Each of these spots contained 6 fmol ACTH. The laser output was directed at each of the sample spots and the laser was fired 50 times at the lowest irradiation setting. The ions detected from this set of laser shots were accumulated as five mass spectra (ten laser shots each), and from that, the mean peak height of the monoisotopic ion, [ACTH+H⁺] at m/z=2465.2 was calculated and plotted with an error of one standard deviation of the mean. The laser irradiation setting was incremented and the analysis repeated (FIG. 22a). This set of data shows the peptide ACTH was in fact present in all samples, but its detection was dependent on the laser irradiation energy, which was interpreted as being indicative of the relative quantity of ACTH in these two samples that had cocrystallized and/or sorbed onto CHCA crystals [28]. The sample with a larger abundance of CHCA crystals offered a greater crystal volume and/or a larger surface area from which peptide compounds could be desorbed, and ion signals for [ACTH+H⁺] at higher S/N were detected at a lower threshold for laser irradiation.

The importance of net charge in the medium during nucleation versus placing an electrode in a medium to raise its potential above ground was investigated. Aliquots of the single component peptide starting solution (1.00 μL aliquot, 319 fmol/μL of ACTH deposited) were pipetted directly onto a MALDI plate and allowed to dry while the MALDI plate was either grounded or biased with a DC potential. Note that in these experiments each aliquot was overall net neutral, and the aliquot spread over a region circular in shape and had a diameter of 1.5 mm once dry. Within the area irradiated by the N₂ laser that was focused to a spot size of 200 μm in diameter, there would have been approximately 6 fmol of material. When the [ACTH+H⁺] signal intensity was measured from these sample spots that were large relative to the laser spot size, the signal intensity increased and then stabilized at a plateau (FIG. 22b) as the area of sample irradiated increased as the laser fluence increased. This was because fresh sample material was accessed as the laser spot size increased with larger aperture sizes (plotted as percentage laser irradiation setting, x-axis, FIG. 22b, inset). If the laser fluence was increased further, the [ACTH+H⁺] signal intensity would eventually decrease because no new cocrystallized MALDI matrix/peptide material would be irradiated and that being irradiated was being consumed. The lack of a separation between laser threshold detection of ACTH in the MALDI-MS of these samples suggests that there was no detectable difference in the two preparations with respect to the CHCA crystal abundance or surface area.

### Example 5.0

### 5.1 Promotion of NaCl Precipitation in Droplets with Net Charge as Intermediaries in the Production of Gaseous Ions in an Electrospray (ES) Ion Source

### 5.1.1 Experimental Procedures

In this Example the following chemical reagents were used: 3.7 MBq ³²P labelled orthophosphate, NaCl, and 20 nm diameter fluospheres (Molecular Probes, Invitrogen Inc., Burlington, ON, Canada). These fluospheres are polystyrene based spheres (density =1.05 g ml⁻¹) that encapsulate ˜180 fluorescein molecules per fluosphere. The composition of all starting solutions from which single droplets were dispensed consisted of a single solute, either NaCl or the fluospheres, dissolved in either 100% distilled deionized water or in 97:3 distilled deionized water:glycerol. The 3.7 MBq ³²P labelled orthophosphate was used only to determine the volume of starting solution dispensed in discrete droplets.

For droplet dispensing a micropipette was used to load a 3-5 μL aliquot of a starting solution into the reservoir of an ink-jet style droplet dispenser as described above. The nozzle of the droplet dispenser was aligned overtop a 5 mm diameter hole cut in a flat electrode, and positioned with a separation distance of 2 mm between the nozzle and the electrode. A dc potential applied to this electrode established an electric field between it and the nozzle of the droplet dispenser. The electric field influenced ion mobility in the volume of liquid that would become the droplet, but only while that volume remained in contact with the bulk liquid inside the reservoir of the dispenser. The induced charge separation within that volume of liquid caused the resultant droplet to have net excess charge as described above. Each droplet passed through the hole in the induction electrode, and into an EDB where it was then trapped and levitated.

For the purpose of measuring the magnitude of the net charge carried by individual droplets, they were dispensed directly onto a metal target plate connected to an electrometer (model 6517a, Keithley Instruments, Cleveland, Ohio). For these measurements, the droplet dispenser and metal target plate were situated inside a Faraday cage. dc potentials were applied to the induction electrode in the range from 100 to 200 V. For induction potentials of 100, 150, and 200 V dc, the induced net charge per droplet were measured to be −135±11, −235±12, and −325±18 fC, respectively, with a starting solution containing 100 mM NaCl in distilled deionized water loaded into the droplet dispenser.

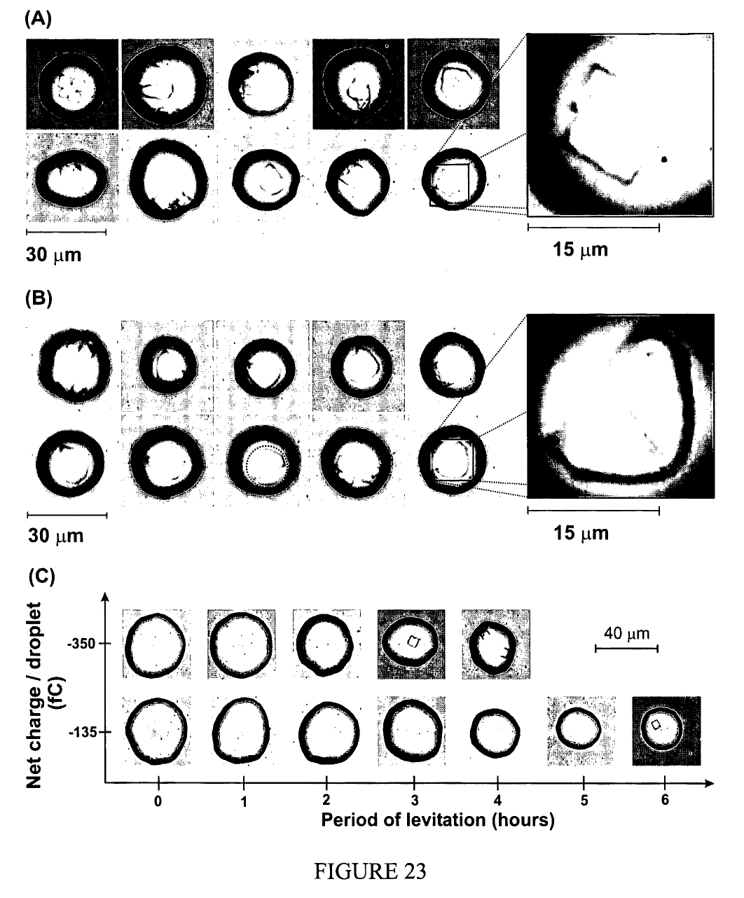

The initial volume of the droplets dispensed was determined by dispensing a starting solution containing 3.7 MBq ³²P labelled orthophosphate directly into a liquid scintillation vial. Radionuclide decay was measured using a liquid scintillation counter (LKB Wallace 1217 RackBeta, Fisher Scientific, Montreal, PQ). Droplets dispensed from the 40 μm diameter orifice had initial volumes of 230±40 μL (average radius 38±2 μm). Droplets dispensed from the 60 μm diameter orifice were measured to have initial volumes of 780±40 μL (average radius 57±2 μm), and this dispenser was used only in the time-lapsed nucleation experiment, the results of which are presented in FIG. 23c. Standard safety procedures for handling radionuclides were implemented during this work.

The EDB used in this Example has been described above. This EDB was assembled using two ring electrodes and two end-cap electrodes. The ring electrodes were fabricated using 1 mm diameter copper wire that was shaped into 2 cm diameter rings. The rings were aligned parallel with respect to themselves and mounted with a separation distance of 6 mm. This pair of ring electrodes was mounted either parallel or tilted at an angle of 15 degrees relative to the end-cap electrodes.

A 60 Hz sine wave, 0-2,500 V_{0-P} was applied to these rings in phase using a Variac-controlled voltage amplifier that had been constructed in-house. The upper end-cap electrode served two purposes in these experiments. It was the induction electrode during droplet dispensing and it was the top end cap for the EDB during droplet levitation. The bottom end-cap of the EDB also served two roles. A dc potential was applied to it to assist in balancing the droplets at the null position of the EDB, and it also served as the target plate onto which the levitated droplets were deposited at the end of each levitation experiment. The null position of the EDB was defined as a point midway between the two ring electrodes when the EDB was viewed from the side, and when the EDB was viewed from the top, that same point was at the center of the ring electrodes. Adjustment of the dc potential applied to the bottom end-cap created an electric field that imparted a force on the droplet causing it to leave the EDB and impact on the target plate. The bottom end-cap was mounted onto a single-axis translation stage to permit precise relocation of the target plate relative to the ring electrodes of the EDB during an experiment.

Each replicate within an experiment commenced with a droplet dispensing event. The droplet flew into the EDB where it was captured and levitated at the null position of the EDB. The volatile solvent used in the starting solution, distilled deionized water, evaporated rapidly from the droplet, typically within 2 seconds of the droplet formation event [129], leaving behind all solutes and solvents of low volatility. Experiments were performed at relative humidity of 38% at which glycerol to water ratio was 85:15 in those droplets that contained glycerol [130]. As soon as the droplet was dispensed, the dc potential applied to the induction electrode was changed to 0 V in all experiments.

For starting solutions that contained 3% glycerol by volume, the droplets that remained levitated following the rapid loss of volatile solvent were stable with respect to Coulomb explosion. For reference, a net charge of −325 fc contained within a 10 μL glycerol-water droplet is only ˜15% of the net charge that such a droplet could retain before undergoing Coulomb explosion. These droplets could be levitated for periods up to 8 hours, and as such, their ejection from the EDB within this period of time necessitated adjustment of the dc potential applied to the target.

The droplets created from starting solutions that did not contain glycerol underwent Coulomb explosion within 2 seconds of the time of their creation. In these experiments, as soon as each droplet was observed inside the EDB, as verified by laser scatter, the amplitude of the ac potential applied to the ring electrodes was reduced from 2500 to 700 V_{0-P}. The reduced ac amplitude caused the droplet to be levitated near the null position of the EDB with an amplitude of motion less than 0.5 mm prior to it undergoing Coulomb explosion. The explosion itself was characterized by a sudden onset of oscillation of the droplet with amplitudes of ±2 mm from the null position of the EDB. [131] Following the Coulomb explosion, the main residue was observed to levitate briefly (<0.5 s) at the null position and then fall to the target plate.

In most experiments, a glass coverslip was positioned on top of the deposition target, and the levitated droplets were caused to deposit onto it. The deposition of droplets onto a glass target greatly facilitated their subsequent characterization by optical microscopy (Motic, B5 Professional, Richmond, BC) and fluorescence microscopy (Zeiss, Axioplan2, Germany).

Initial experiments involving levitated droplets having net charge were designed to not allow the droplets to undergo Coulomb explosion. Individual droplets were created having either −135 or −325 fC of net charge using a starting solution of 285 mM NaCl in 97:3 water:glycerol. Within seconds of the droplet generation event, the droplet volume had decreased to ˜10 μL. The droplets were levitated for a total of 5 minutes prior to being deposited onto a glass coverslip. NaCl precipitation in levitated droplets was selected for the initial studies because of the relative simplicity of the system, the certainty with which the identity of the ions of the droplet's net excess charge (ions_{NEC}) were Cl⁻_{NEC} because of their high abundance relative to other impurity electrolytes and compounds in the starting solution, and because NaCl in biological sample types often suppresses analyte ion signal intensities as measured by mass spectrometry. [132].

Alteration of the magnitude of the net charge imparted onto droplets had a profound effect on the morphology of NaCl precipitates formed during levitation. When droplets contained net charge of −135±11 fC, the resultant precipitate (NaCl₍ₛ₎) in 23 droplet residues were cubic, except one in which the NaCl₍ₛ₎ had a cube-like morphology (FIG. 23A). When the magnitude of the net charge per droplet was increased to −325±18 fC, NaCl₍ₛ₎ observed in 16 out of 19 droplet residues had cube-like morphology (FIG. 23B). Also of note is that the radii of curvature of the dome shaped portion of the precipitates were similar, as denoted by a dotted curve in FIG. 23B. The similarity in the radius of curvature suggested that nucleation was initiated in the droplet-air interface, but certainly the initial growth of the precipitate took place in the diffuse layer at the droplet-air interface which imposed geometrical constraints. Further growth of the precipitate allowed it to branch away from the droplet air-interface into the interior of the levitated droplet. The final size of the precipitate in the droplet residues suggests that the levitated droplets' spherical shape was deformed in the latter phases of growth of the precipitate. The macroscopic appearance of the NaCl₍ₛ₎ obtained when the polarity of the droplet's net charge was positive was similar. Because the crystalline nature of these precipitates have yet to be determined, the NaCl₍ₛ₎ will continue to be referred to here simply as a precipitate.

In 95% of these droplet levitation trials, NaCl₍ₛ₎ was observed to have formed during the period of levitation, and no new NaCl₍ₛ₎ was observed during the post-levitation period when the droplet's residue was in contact with the surface of the glass slide for up to 4 hours after the time of deposition. In the remaining 5% of the trials, no NaCl₍ₛ₎ was observed to have formed in the droplet while it had remained levitated. In these situations, precipitates that did form after deposition were a result of heterogeneous nucleation at the interface between the glass slide and the droplet. The growth of the crystals at the droplet-glass interface resulted in visibly different morphology for those precipitates relative to NaCl₍ₛ₎ that had formed within the levitated droplet, which eliminated ambiguity between precipitates that had formed in the levitated droplets versus in droplets that were in contact with a glass surface.

Comparison of the residues in the images shown in FIGS. 23A and 23B suggest that the net charge from the 2×10⁶ Cl⁻_{NEC} (−325 fC per droplet, FIG. 23B) caused the formation of NaCl precipitates having a region of curved morphology. This suggests that NaCl nucleation in the diffuse layer was promoted to the extent that nucleation in the neutral droplet core, where there was 4.3×10¹³ Na⁺ Cl⁻ ion pairs, was out-competed. Conversely, in the levitated droplet residues that had 1×10⁶ Cl⁻_{NEC} (−135 fC, FIG. 23A), one precipitate in one of the 23 trials was a dome-shaped NaCl solid observed, suggesting that in these droplets, nucleation commenced in the bulk. It is believed that the location of initial nucleation in a levitated droplet can be influenced to occur in either the diffuse layer at the droplet-air interface or in the droplet's interior by control of the magnitude of the net charge induced onto the droplet.

These trials of NaCl₍ₛ₎ formation in levitated droplets were repeated using the 285 mM NaCl in 97:3 water:glycerol starting solution, except that the droplets were levitated in an atmosphere of N₂ by purging the levitation chamber with ultrahigh purity N₂. The purpose of the N₂ atmosphere was to reduce the possibility that the nucleation being observed in these levitated droplets was due to electrostatically charged dust particles suspended in the atmosphere and that had adsorbed onto the levitated droplets.⁵² The morphology of the NaCl precipitates that formed in the levitated droplets in this experiment, were similar to that already described and presented in FIGS. 23A and 23B, indicating that nucleation because of suspended particles in the atmosphere was not a factor. Note that the relative humidity in these trials was reduced to 2%.

The results presented in FIGS. 23A and 23B were obtained using a starting solution with a very high concentration of NaCl (285 mM) in the starting solution. There existed the possibility that the precipitates observed had their nuclei formed as a result of droplet cooling during the period of time (<2 s) that the water evaporated, [133] and for a brief period following that while the droplet temperature re-equilibrated with the temperature of the chamber. To address this question, a starting solution containing 69 mM NaCl in 97:3 water:glycerol was loaded into the droplet dispenser. Droplets dispensed from this solution and levitated for 5 minutes exhibited no observable precipitates. Using this starting solution, each iteration of the experiment commenced with trapping and simultaneous levitation of a population of 40-45 similarly dispensed droplets. 4 droplets were ejected from the EDB at the end of each hour of levitation and deposited onto a glass slide. Representative images of droplet residues obtained from two trials of this experiment are presented in FIG. 23C. The magnitude of the net charge on each droplet in the first population was −135 fC (trial 1), and in the second population, −350 fC (trial 2). For the droplets having −135 fC of net charge, NaCl(s) was first observed 6 hours after the initial droplet dispensing events. Notice the reduction of the size of the droplet residue with time in this series is visual evidence of solvent evaporation from the levitated glycerol-water droplets. In contrast, NaCl₍ₛ₎ was observed after only 3 hours of levitation within the droplets that had −350 fC of net charge, indicating that nucleation had occurred at a lower concentration of Na⁺Cl⁻. These results indicate that the nucleation and growth of a NaCl precipitate was promoted as a result of an increased magnitude of droplet net charge, and hence clusters in droplets generated by an ES could be forming at lower solute concentrations than expected based on homogeneous nucleation. These results also suggest that the initiation of nucleation due to cooling of the droplets is not be a factor, since the temperature of the droplets had enough time to equilibrate with ambient temperature. However, the possibility that the microcrystals were formed in the cooled droplets within the first few seconds of their lifetime, and then persisted for hours prior to reaching conditions in the droplet that allowed their growth, cannot be ruled out.

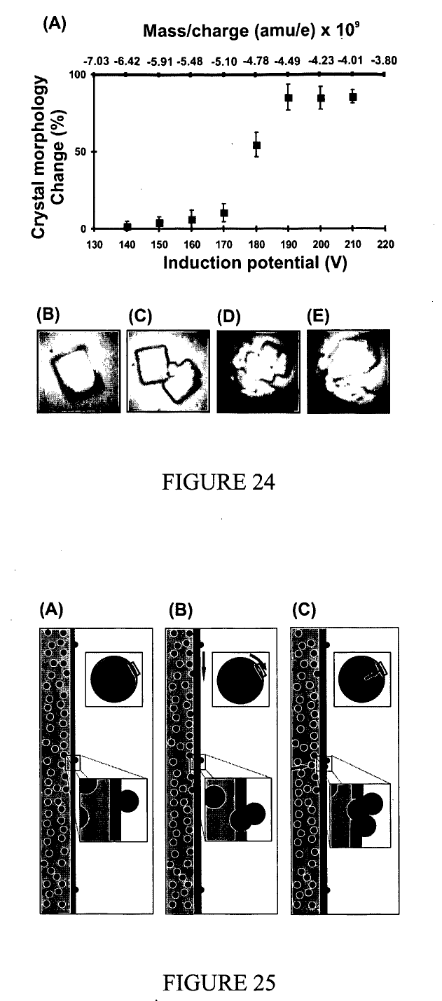

Possible factors responsible for these observations concerning NaCl nucleation and precipitate growth are the magnitude of the droplet's net charge and the relative rates of solvent evaporation from the droplets. [134] These two factors are related because droplets with higher net charge have higher mobility, and differential mobility of droplets levitated in the EDB would lead to proportionally different rates of solvent evaporation. An indication of the relative role that these two factors have in determining the nucleation and growth of the precipitate was learned from the following experiment. The starting solution consisting of 285 mM NaCl in 97:3 water:glycerol was re-loaded into the droplet dispenser and droplets dispensed using different dc potentials applied to the induction electrode. Each data point in FIG. 24A was obtained by dispensing 50 droplets at each of the induction potentials indicated, and then the number of droplets having NaCl precipitates with a region of curved morphology was plotted as a percentage. Note that typically only one precipitate per droplet formed. The insets in FIG. 24 are representative images of a (B) a cubic precipitate and (C,D,E) precipitates having a region of curvature (i.e. deviation from cubic morphology). This interpretation of the NaCl₍ₛ₎ morphology indicates a threshold magnitude of net charge for the droplets, plotted as droplet m/z on the top x-axis in FIG. 24A. The threshold value in m/z was 4.×10⁹. Below this threshold of m/z, was the onset of ion-induced nucleation in these glycerol-water droplets.

In addition to the results plotted in FIG. 24A, another set of trials were performed during which the amplitude of the AC was adjusted so that in any one trial, the levitated droplets had amplitudes of motion ˜2 mm for all trials, and hence their relative velocities in the EDB were similar. This trial was performed because the existence of a threshold for observation of precipitates having curved morphology in FIG. 24A suggested that the net charge was by comparison more important than any differences in the rate of solvent evaporation from the levitated droplets. Within the EDB, a levitated droplet's motion can be described using an a-q space analogy of the Paul trap with the correction for heavy dampening due to droplet-buffer gas collisions at atmospheric pressure. [135] In the absence of large contributions of higher order fields, a droplet's mobility with increasing net charge should have a predictable, and likely linear effect on the rate of solvent evaporation. Hence the reason for adjusting the electric fields used to levitate the droplets so that droplets having different net charge (in different replicates) had similar amplitudes of motion, and therefore velocities in the EDB. Similar results were obtained to that presented in FIG. 24A. Had the rate of solvent evaporation been the dominant factor in the nucleation and growth of the NaCl precipitates, a near linear relationship between the morphology of the NaCl precipitates and the droplet net charge would have been expected.

Two prior independent studies investigated nucleation of electrolytes in electrodynamically levitated aqueous droplets. The authors of these reports concluded that small variations of the magnitude of the net charge did not observably affect nucleation. [136,137] The median m/z for the aqueous droplets used in those prior studies are estimated to have been 3.4×10^{9 52} and 7.4×10¹⁰. [137] Comparison of the Cohen results to that presented in FIG. 2 suggests that properties of the solvent, such as surface tension, viscosity, and dielectric constant (glycerol 63.4 dynes cm⁻¹, 954 cp, 42.5 and water 73.05 dynes cm⁻¹, 0.89 cp, 78.54, respectively), could be important parameters in affecting NaCl nucleation dynamics that in turn affect the threshold droplet m/z value for observation of nuclei having curved morphology in levitated aqueous versus glycerol droplets. For reasons of relevance to the planet's climate, nucleation in droplets suspended in the atmosphere has also been studied extensively.[138,139] While there is consensus regarding the nucleation as being heterogeneous, Duft et al. have argued the process is volume dominated, but others have argued that the process is surface stimulated.[140,141]

Synergistic macroscopic and nanoscopic interactions are likely responsible for the promotion of nucleation (FIG. 25). At high magnitudes of net charge, nucleation commenced at an ion_{NEC}. Ions_{NEC} were calculated to exist in the diffuse layer of droplets with net charge.[142] Possible interactions between one such ion and other neighbouring ions are depicted in FIG. 25. What is driving those interactions? Experimental factors that are most certainly involved in promoting the nucleation of dissolved solids are the magnitude of the levitated droplet's net charge and, not having completely ruled out the effect of the rate of solvent evaporation. Further studies of this phenomenon using varied solutes and solvents, and varied EDB electrode geometries to investigate the possibility of higher order fields contributing to these observations, are underway.

The promotion of nucleation within droplets having net charge was observed to be dependent on the magnitude of that net charge. The inventors term this phenomenological result ion-induced nucleation in solution. Ion-induced nucleation in the gas phase is the promotion of cluster growth by introducing discrete charged entities that act as nuclei for condensation of vapours, and it has been studied extensively since Wilson reported the phenomenon over one hundred years ago.[143,144] Since then, it has been learned that gas phase ion-induced nucleation occurs because there is a reduction in the free energy required for nucleation and growth relative to that in the absence of an ion.[145-147] For instance, the low pressure synthesis of diamond is rendered favourable over graphite because gaseous C⁻ reduces the critical radius for crystal nucleation.[148] and the net charge of the resulting cluster promotes further growth of the diamond nucleus.[149]

Ion-induced nucleation in solution could have implications for natural phenomena such as nucleation in suspended atmospheric particles, particularly those that contain net charge such as sea salt droplets.[150-151] With further experimental characterization and a theoretical description of this phenomenon, it could find utility as a new tool for laboratory studies of crystal nucleation and growth, or as a medium for the production of nuclei that would be used to seed crystal growth in secondary vessels.¹⁹ With these considerations in mind, further development of the methodologies reported herein to enable quantitative characterization of chemical processes that occur in media with net charge are being pursued.

Recall that droplets generated in an ES have an estimated initial m/z of 1×10⁹. We suggest that the m/z of droplets generated by an ES are either at or below the threshold for nucleation at the instant of their formation, and if not, they certainly reach that threshold as suggested by the observation of clusters ES-MS. Furthermore, the promotion of nucleation was observed at magnitudes of net charge less than that required for droplet Coulomb explosion. It is very interesting to point out that in an ES, the pathway that leads to the production of gaseous ions involves the production of droplets having higher relative net charge because of “charge enrichment” in the matter ejected by a droplet Coulomb explosion event.[152,153] Those droplets all existed in a strong electric field, meaning that their increased mobility will also lead to increased rate of solvent evaporation. Hence, this phenomenon of ion-induced nucleation and factors that promote it; magnitude of net charge and possibly also local fluctuations of solute concentrations because of rapid solvent evaporation, all work to promote nucleation and growth of the precipitate. For instance, it could be argued that the recent results from lavarone et al. were in fact a clever example of how to apply ion-induced nucleation in solution to reduce the concentration of the interfering species, while allowing the sought for analyte compounds to remain in the solution phase in the time preceding droplet Coulomb explosion.[154] Interestingly, Julian et al. proposed that magic numbers of serine clusters form by self-assembly in the droplet bulk and later become ionized in the diffuse layer at the droplet-air interface of droplets with net charge.[155] but further studies of this phenomenon by Takats et al. prompted them to speculate that the most abundant serine cluster, the octamer, could itself be formed at droplet-air interface.[156] Furthermore, images of droplet residues reported by Hanton et al. that were obtained by introducing solutions containing organic acids commonly employed as a matrix in MALDI-TOF, such as 2,5-dihydroxybenzoic acid and dithranol, to an ES source for the purpose of preparing films of these compounds.[157] Using scanning electron microscopy to study the resultant films, spherical residues were observed, as expected based on complete evaporation of solvent from the droplets, but, many of those spheres were hollow. That observation by Hanton et al. suggested to us that the dissolved solids were being caused to preferentially crystallize at the droplet-air interface, but not in the core of the droplet, and that subsequent growth of those nuclei in the diffuse layer at the droplet-air interface was favored.

Based on the observations of ion-induced nucleation in solution reported in the previous section, and the clusters that are readily observed in ES-MS, there can exist entities that range in size from macroscopic solids to clusters and nuclei, in the diffuse layer at the droplet air interface of droplets with net charge dependant on the concentration and solubility of the solutes in the starting solution. Do such entities affect the discharge dynamics of Coulomb explosion? To address this question through experiment, we developed a method that allowed the materials separated by Coulomb explosion of a single droplet to be collected at different locations of a target, thereby enabling their differential characterization. In the absence of nuclei or clusters (or at least in the absence of appreciable quantities of nuclei), Cohen et al. measured no difference in the discharge dynamics of droplets generated in an ES when starting solutions consisting of NaCl at 1, 10, and 100 μM were introduced to the ionization source.[152]

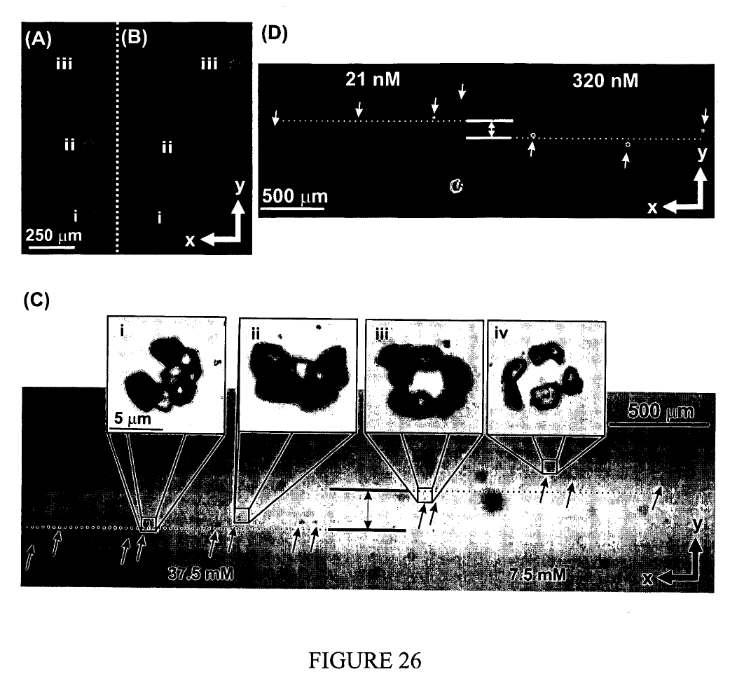

The alignment of the ring electrodes of the EDB were changed from perpendicular relative to the target plate, to an angle of 15 degrees relative to the target plate for the following experiments. The electric fields in this configuration of the EDB introduced a force onto the droplets being ejected from the EDB, which caused droplets of different m/z to land on the target at different locations. As a first demonstration of this, droplets were dispensed with a starting solution containing 320 nM fluospheres in 97:3 distilled water:glycerol. One droplet was dispensed at each of the following induction potentials, identified as (i) 100, (ii) 150, and (iii) 200 V dc in FIGS. 26A and 26B. One of these droplets at a time was levitated in the EDB for 5 minutes. The resultant glycerol-water droplets, which had not undergone Coulomb explosion, were deposited using the same dc potential applied to the target plate. The mass-to-charge of the droplets were estimated as 7.8×10⁹, 4.8×10⁹, and 4.2×10⁹, respectively. When the fluosphere emission from within these droplet residues on the target plate were imaged and the three circular-shape residues were observed in a straight line on the target plate and the center-to-center separation between any two residues was 350 μm (FIG. 26A). The y-axis is defined as being parallel to the line of droplet residues formed on the target. This experiment was repeated but with the target plate being translated in the direction of the x-axis between droplet deposition events to provide further confirmation of the identity of the droplets (FIG. 26B). Similar separation in the y-direction for the location of the droplet residue deposition sites on the target were obtained, and in addition, this provided additional confirmation of a relationship between the location of droplet deposition on the target and the droplet's m/z. The small changes to the electric field that are introduced when the target plate is translated relative to the ring electrodes are known to introduce random error into the site of deposition on the target of no more than ±5 μm.[158]

Having established the directionality to the m/z scale of the relative location for the droplet landing on the target, the following experiment was performed. Two starting solutions containing NaCl at 7.5 mM and 37.5 mM in water were used. A 200 V dc potential was applied to the induction electrode. Within experimental error, these droplets from either solution had the same initial volume and net charge at the moment of dispensing. Individual droplets were trapped and levitated in the EDB until they were observed to undergo a rapid oscillation in amplitude, which is characteristic of a droplet undergoing Coulomb explosion.[131] After the main residue had landed on the target, the target plate was translated relative to the ring electrodes along the x-axis for the purpose of isolating main residues between successive iterations of this experiment. The main residues following Coulomb explosion of individual droplets are denoted by the white arrows in FIG. 26C. The mean y-axis position of the main residues on the target was dependent on the initial concentration of NaCl in the starting solution. When the 37.5 mM NaCl starting solution was used, the main residues had higher m/z than the main residues resultant when the 7.5 mM starting solution was used.

The equation (qe)²=64π² ∈ₒγR³ predicts the condition for onset of Coulomb explosion, where γ is the surface tension of a droplet of radius R and having q net elementary charges.[152] According to this equation, the droplets studied here would have shrunk to a radius of ˜9 μm when the explosion occurred, at which point the concentrations of NaCl would have been approximately one order of magnitude below its solubility limit. Hence, we suggest that NaCl precipitates could have existed in the droplets at the time of Coulomb explosion as a result of ion-induced nucleation. It is however known for certain that there were NaCl precipitates in the main residue following Coulomb explosion, and the number of precipitates (in the absence of glycerol) suggests that nucleation initiated at numerous sites. The difference in solute mass could account for the relative deposition locations of the main residues on the target (FIG. 26C). Based on this information, we cannot conclude that the discharge dynamics of the Coulomb explosion was solely responsible for the difference in m/z of the main residues, although, the residues were not completely dried upon deposition as viewed by optical microscopy. Nevertheless, we note in the two insets iii and iv FIG. 26C, the numerous NaCl precipitates formed a full circle, whereas in insets i and ii in FIG. 26C, the NaCl precipitates were distributed in a semicircle. That leaves the door open for speculations such as the differential m/z of the main residues shown in FIG. 26C was also a function of the explosion discharge dynamics being affected by macroscopic solids existing in the diffuse layer of the levitated droplet.

For further investigation, another experiment involving two starting solutions consisting of 21 or 320 nM fluospheres in water were used. Fluospheres were used because at similar concentrations, the size of the NaCl(S) would not be decipherable on the target, and the fluospheres are themselves large entities as measured on a molecular scale. Hence the fluospheres are near ideal test solutes to investigate the possibility that the presence of 20 nm diameter entities affect the discharge dynamics of a Coulomb explosion. Droplets were dispensed with one of the two starting solutions loaded into the droplet dispenser and a dc potential of 200 V applied to the induction electrode. The initial volume and net charge of these droplets were the same within experimental error. Droplets were levitated individually until they underwent a Coulomb explosion, as verified through visual observation of the levitated droplet. Following the explosion, the main residues were observed to have been levitated briefly in the EDB prior to falling out of the EDB and onto the target. The target was translated relative to the ring electrodes of the EDB along the direction of the x-axis to permit isolation of each main residue prior to dispensing another droplet. Fluorescence emission from the fluospheres retained in seven main residues are observable in FIG. 26D. The deposition location on the target of seven main residues, indicated by the white arrows in FIG. 26D, were dependent on the initial concentration of the fluospheres in the starting solution. The region of bright fluorescence emission in the center of this image was from a droplet that had passed directly through the EDB without having been levitated.

At the time these droplets underwent a Coulomb explosion, the total mass of fluospheres in any of these droplets was estimated as <2 ppm by mass. Hence, the different concentration of fluospheres in these droplets cannot account for the different m/z of the main residues that are depicted in FIG. 26D, and we believe that these residues indicate differential discharge dynamics brought about because of the different concentrations of the 20 nm diameter fluospheres existing in the diffuse layer of the droplet when it underwent a Coulomb explosion. The presence of those entities effected the ejection of comparatively less mass, or more net charge, or a combination of these two factors. Work is in progress to quantitate the fluorescence emission from the main residues to learn which of these processes occurred. If future experimental results indicate that a higher fraction of net charge is ejected as a result, it could be argued that the efficiency of transfer of clusters and solids from within the droplets to the gas phase is proportional to the size.

The promotion of NaCl nucleation in levitated glycerol droplets was found to be dependent on the magnitude of the net charge in the diffuse layer at the droplet-air interface. This phenomenon is being termed ion-induced nucleation in solution. Some of the potential implications of this result on the performance of ES-MS when characterizing solutes in solutions containing high electrolyte concentrations were discussed. It was speculated that the existence of precipitates and nuclei, such as clusters, in the diffuse layer at the droplet-air interface preceding the explosion influences the discharge dynamics. A first measure of the m/z of the main residues resultant from a Coulomb explosion when the droplets contained 20 nm size entities (fluospheres) indicated that there was a fluosphere concentration dependence on the discharge dynamics.

### Example 6.0

### 6.1 Ion-Induced Precipitation of NaCl, CHCA, THAP and Samples of D and L Serine in Levitated Droplets Having Net Charge

Several experimental factors regarding precipitation of NaCl in electrodynamically levitated droplets of water/glycerol have been characterized. These droplets carry net elementary charge which is comprised of a population of ions of a single polarity that are in excess of counterions in the droplet. In this Experiment the onset of ion induced precipitation of NaCl was studied as a function of droplet size, surface net excess charge density, viscosity of the droplet, and levitation time at which nuclei were observed to have formed in the levitated droplets as a function of the magnitude of the net charge. The promotion of precipitation of two organic compounds, α-cyano-4-hydroxycinnamic acid (CHCA) and 2,4,6 trihydroxyacetophenone (THAP) was also demonstrated in levitated glycerol droplets as a function of the droplet net charge magnitude. Lastly, investigation of the precipitation of stereoisomers of serine was performed in bulk media that did not have net charge, and in levitated glycerol droplets that possessed net charge. The onset of precipitation for samples of D versus L serine in the levitated droplets of varied net charge was observed to be different. Collectively, these results are examples of effecting a degree of control over the precipitation of dissolved solids. The inventors repeatedly observed in experimental results that indicated the magnitude of the net charge altered the solubility of a dissolved solid, or solids, contained in a levitated droplet. In all cases, decreased solubility for dissolved solids was observed when the magnitude of the net charge on the levitated droplet was increased, likely because of heterogeneous nucleation on or adjacent to an unpaired ion in the levitated droplet.

### 6.2 Experimental Characterization of Ion-Induced Nucleation Phenomenon in Solution Using Sodium Chloride as a Model Solute

As discussed above, the inventors have demonstrated ion-induced nucleation to the condensed phase in charged glycerol-water droplets levitated in an electrodynamic balance 12 (EDB). In the case of sodium chloride, ion-induced nucleation was manifested in morphological change, from a cubic shaped crystal to one that contains curved morphology. The promotion of nucleation was onset at a mass-to-charge ratio of 4.78×10⁹ amu/e. While this phenomenon has been observed for several organic and inorganic compounds, the inventors have used sodium chloride as a model solute due to the relative simplicity of the system and the certainty with which the identity of ions carrying the net excess charge (ions_{nec}) were either Cl⁻ or Na⁺.

As described above, droplets were dispensed from a starting solution containing the solute of interest and a net charge was imparted on each droplet. The origin of the ions that carried the net charge were either electrolytes added to the starting solution or generated by electrolysis. These ions_{nec} were localised in a diffuse layer at the droplet-air interface, where they collectively formed an electric potential that diminished to null in the centre of the droplet. Each droplet was captured and levitated in the EDB 12. The volatile solvents evaporated leaving behind a glycerol-water droplet of 11±1 pL that retained the solutes and, in some cases, became supersaturated. Subsequently, droplets were simultaneously deposited on a glass slide and the droplet residues were examined with optical microscopy.

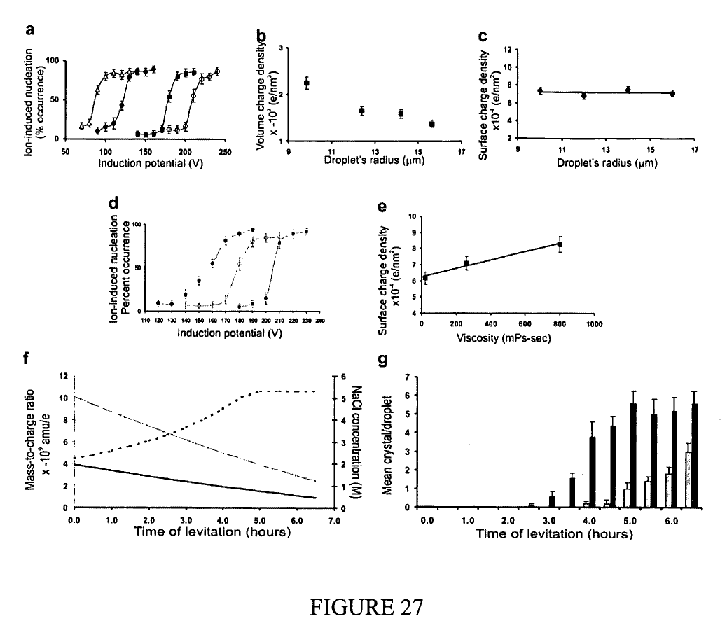

In this Example an experiment was performed to investigate whether the onset of ion-induced nucleation in a droplet is dependent on the surface charge density and hence occurs at the droplet-air interface or on the volume charge density, hence occurring virtually anywhere in the droplet. The inventors varied the volume of the droplets for up to four-folds with corresponding radii 10 μm to 16 μm, while the sodium chloride concentration remained constant and monitored the percent occurrence of ion-induced nucleation as a function of induction potential (FIG. 27a). The surface charge density remained constant at the onset of nucleation regardless of the droplet's size whereas volume charge density was reduced (FIG. 27b,c). In both hypotheses, the inventors assumed homogeneous charge distribution either at the surface or throughout the volume of the droplet, respectively. These results suggest that ions_{nec} reside at the surface of the droplet, as previously reported, and that the promotion of nucleation occurred at a surface charge density of-7.22×10⁻⁴ e/nm². This charge density is 3 to 4 orders of magnitude lower than that previously reported in studies where nucleation was promoted at a charged surface due to and externally applied electric field. That suggests another mechanism by which nucleation is promoted in droplets with net charge, such as the incorporation of a surface ion_{nec} in a nucleus acting as a seed to accelerate the growth.

Next, the inventors investigated the effect of the droplet's physical properties on the surface charge density required for ion-induced nucleation to occur, by changing the glycerol/water composition with sodium chloride concentration remaining constant and monitoring the percent occurrence of ion-induced nucleation as a function of induction potential (FIG. 27d). The surface charge density required linearly increased with an increase in the viscosity of the solvents over the range of 20 to 800 mPa-sec (FIG. 27e). Again, the inventors assumed that all ions_{nec} resided at the droplet's surface. The increase in the required surface charge density could be attributed to unequal charge distribution, between the droplet's core and surface, as a consequence of changing the medium's composition hence the droplet's conductivity. It is interesting to note the consistent decrease in the slope of the curves in FIG. 27k, offering the possibility to use charged droplets as a tool for the promotion of nucleation with more control over the onset of ion-induced nucleation in less viscous mediums such as water.

The inventors then levitated two populations of water-glycerol droplets containing the same concentration of NaCl, which was low enough not to induce crystal formation at least in the first two hours of levitation. The net charge imparted on both populations was −135 fC and −350 fC, respectively. While the droplets were levitated, the glycerol's slow evaporation gradually reduced the mass-to-charge ratio of the droplets and simultaneously increased the NaCl concentration (FIG. 27f). The population of droplets with −350 fC had higher surface charge density than required for the onset of nucleation while the other population did not Observable crystals appeared after −3 hours of levitation in the first population with NaCl concentration −3.4 M, while crystals were only observed after 5.5 hours in the population with initial net charge of-1 35 fC, at a concentration of 5.3 M, which is the solubility limit for NaCl in water (FIG. 27g). These results suggest heterogeneous nucleation of NaCl in the first population and hence the reduced concentration at which nucleation occurred.

The effect of ion-induced nucleation on the morphology of sodium chloride crystals also depended on the droplet's solvent composition. Curved crystals were more predominant when the water content was ≧10% whereas below that the growth of NaCl crystals was hindered by the high viscosity and the crystals assumed cubic or amorphous shaped and rarely exceeded ˜7 μm in dimension. Below the requisite surface charge density, these crystals were predominantly found in the middle of the droplet's residue which indicates their formation in the core of the droplet, while upon the occurrence of ion-induced nucleation, reproducible homogeneous distribution of small crystal was found on the periphery of the deposited droplet residues in the case of low water content in the droplet.

### 6.3 Observation of Ion-Induced Nucleation in Solution of Organic Compounds

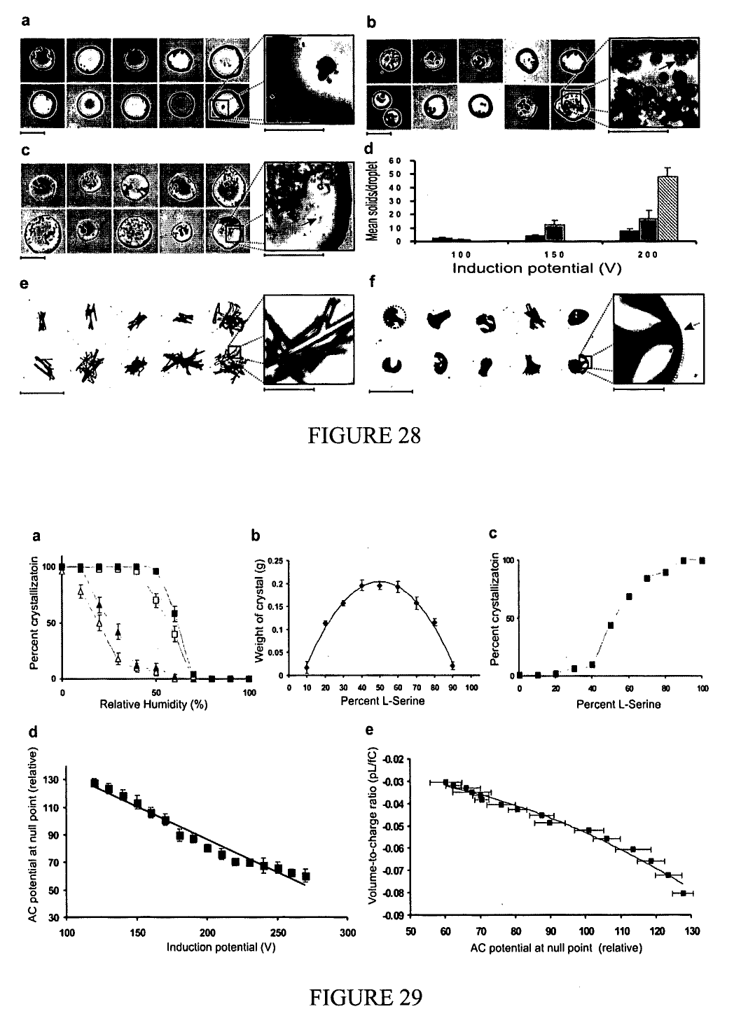

Although the onset of ion-induced nucleation depends mainly on the droplet's properties, this process had different effects on different solutes. In levitated droplets that contained α-cyano-4-hydroxycinnamic acid (CHCA) and that had a net charge of −135±11 fC, only solids >3.5 μm in dimension were observed (FIG. 28a). In addition, CHCA solids that were 1 to 3.5 μm in dimension were observed in droplets in which the net charge had been increased to −235±12 fC (FIG. 28b). An increase in the net charge to −325±18 fC per droplet resulted in the further production of 20-70 CHCA crystals that were smaller than 1 μm in dimension, indicative of a nucleation shower (FIG. 28c). The abundance of CHCA solids >3.5 μm was unaffected by the magnitude of the net charge on the droplets, since they formed due to the solute's supersaturation in the droplet's core (FIG. 28d).

In the case of THAP, the promotion of nucleation at the surface of the droplet manifested in bundled cylindrical crystals with curved morphology (FIG. 28f). It is apparent that after the initial nucleation process, crystals grew either along the curved surface of the droplet or towards its core. On the other hand, when the surface charge density was not high enough for the onset of ion-induced nucleation, randomly organized rod shaped crystals formed sometimes penetrating out of the droplet's surface as indicated by their length (FIG. 28e). The curved portion of the crystals in FIG. 28f points towards the possibility that the charged droplet's surface could act to promote crystal growth.

### 6.4 Preliminary Results Showing the Utilization of Ion-Induced Nucleation in Solution to Differentially Precipitate Chiral Enantiomers of Serine

Starting solutions containing serine in water were levitated. No glycerol was used since serine seems to act as a surfactant that allows the levitated droplet to retain high enough quantity of water in the EDB in equilibrium with the ambient relative humidity. Hence, by controlling the relative humidity, one could control the final volume of the droplet and, consequently, the concentration of serine in the droplet.

Solutions of D- and L-serine were dispensed and, droplets were levitated at various relative humidities. The droplets were subsequently deposited on a glass slide and the percentage of droplets were the precipitation of serine was recorded (FIG. 29a). L-serine had a higher tendency to precipitate in droplets with net charge (i.e. it precipitated out of solution at lower concentrations than D-serine). The greatest difference between the chiral enantiomers was at 40% relative humidity. Shown in FIG. 29a are droplets with D- and L-serine in both low and high net charge (i.e. droplets created at 100 and 200 V induction potential, respectively.) The effect of varying the net charge is not as apparent as the effect of varying the enantiomeric form. However, it is possible (from the above experimental results) that 100 V of induction potential would create sufficient surface charge density for a water droplet in order to promote ion-induced nucleation of serine. Lower net charge has not been tested to determine whether D- and L- would behave differently.

Pure L- and pure D-serine crystallization occurs at >3M concentration. However L- and D-mixtures of serine has been observed to crystallize at ˜1.5 M. This is a known phenomenon where the crystal building block is composed of both L- and D-serine instead of each alone. This mixed solution could have a potential to precipitate at lower concentration than the pure form. Hence, 9 solutions of D- and L-serine mixtures were prepared whereby the percentage of D- increased from 10% to 90%. Crystallization occurred on all 9 solutions at different rates. Samples were centrifuged, and the supernatant decanted and the peletted precipitates were weighed (FIG. 29b). The curve shown in FIG. 3c is clearly symmetric where most precipitation occurred at the 50:50 mixtures of D- and L-.

When the same experiment was repeated in levitated droplets, different patterns of crystallization occurred whereby the symmetry of precipitation of serine enantiomers was broken (FIG. 29c). The starting solutions were at 0.75M mixtures of D- and L- serine as explained above, to prevent premature crystallization in bulk. When droplets were levitated, a certain amount of water evaporated and the percentage of droplets where precipitation of serine occurred was recorded at each solution composition. This experiment was performed at 40% relative humidity, which is the humidity at which there is the greatest difference between the crystallization patterns of D-serine and L-serine. The induction potential used to create all the droplets was 200V.

Finally, in order to calculate the volume of the droplets and hence the concentration of serine in the droplets as a function of humidity, an experiment was performed on the EDB with glycerol droplets as a standard. 3% glycerol solution was dispensed to create droplet of which we know the mass to charge ratios. The charge was varied and the magnitude of the AC field required to center the droplet in the null point of the EDB was recorded as a function of induction potential (FIG. 29d). Then serine droplets could be levitated at a fixed induction potential but different humidities, and the magnitude of the AC field required to center them in the null point of the EDB recorded. Knowing the net charge imparted on the droplets, it would be possible to measure the change in mass hence volume of these droplet from the chart in FIG. 29e.

### 6.5 Materials and Methods

The following materials and methods were used in this Example.

Starting solutions. The preparation of the starting solutions used to create the droplets for these experiments were as follows: i) 1.1, 2.2, 3.3, or 4.4 mg of NaCl, 4, 8, 12, or 16 μL of glycerol, 396, 392, 388, or 384 μL of distilled deionized water, to create droplets with radii of 10, 12, 14 and 16 μm respectively. For the slow evaporation experiment, the starting solution composition was as follows: (1.1 mg) 28 μmoles of NaCl, 12 μL of glycerol, 388 μL of distilled deionized water.

Droplet dispensing. A 5 μL aliquot of a starting solution was delivered to the reservoir of an ink-jet style droplet generator (model MJ-AB-01-60, Microfab, Piano, Tex., USA). The separation between the induction electrode and the nozzle of the droplet generator was 2 mm. The potential applied to this electrode determined the magnitude of the net charge induced onto each droplet. The net charge on individual droplets were measured by dispensing droplets through a 2.5 mm diameter hole in the induction electrode onto a metal plate connected to an electrometer (model 6517a, Keithley Instruments, Cleveland, Ohio). For induction potentials of 100, 150, and 200 V DC, the induced net charge was −135±11, −235±12, and −325±18 fC respectively. The initial volume of the droplets dispensed was determined by dispensing a starting solution containing 3.7 MBq 32P labeled orthophosphate directly into a liquid scintillation vial. Radionuclide decay was measured using a liquid scintillation counter (LKB Wallac 1217 RackBeta, Fisher Scientific, Montreal, PQ). Droplets had an initial volume of 400±20 μL (average radius 45±2 μm).

Droplet levitation. The droplets passed through the hole cut in the induction electrode and into a double-ring electrodynamic balance (EDB). The volatile solvents evaporated within 5 seconds of their formation, and the volume of the levitated droplets shrank to 11±1 μL (average radius 14 μm). The droplets retained the net charge and the solution became supersaturated. Coulomb explosion 27 was not encountered since the net charge increased linearly with the increase of applied induction potential (unpublished results). Droplets were trapped and levitated in the EDB for 5 minutes, and then deposited onto a plate by applying an attractive DC potential to that plate 28. To determine the condition at which NaCl nucleation in levitated glycerol droplets occurred, each experiment involved the generation and levitation of a population of 30 identical droplets, four of which were deposited at 1 hour intervals for a period of 7 hours in total.

Optical microscopy of droplet residues. A t-test was performed on the diameter of 50 droplet residues measured using a calibrated optical microscope (Motic, B5 Professional, Richmond, BC) for three different magnitudes of droplet net charge; −135 fC, −240 fC, and −350 fC. All droplet residue diameters were similar with 99% confidence level. In 95+% of the droplet levitation trials, crystals were observed to have formed during the period of levitation and no new crystals were observed to have formed in the droplet residue while in contact with the surface of the glass slide up to 4 hours after the time of deposition. In the remaining trials, no crystals were observed to have formed in the droplet residue while it had remained levitated. In these situations, crystals that did form after deposition were a result of heterogeneous nucleation at the interface between the glass slide and the droplet, and their morphology was different than the crystals that formed within the levitated droplet. Similar results were obtained for droplets with net positive charge.

Controlling the size and composition of the droplet. The size of the droplet is controlled through the percent glycerol in the starting solution. In this experiment, starting solutions containing 14% glycerol were used. NaCl concentration in the starting solution was changed accordingly to keep the final solution in the water-glycerol droplet constant. The glycerol-water composition of the droplets was controlled via changing the humidity. Droplets were levitated at 10%, 30%, and 60% relative humidity which corresponds to 3, 10, and 31% water by volume. The viscosities of these solutions were 21, 259, and 800 mPa-sec.

### REFERENCES

### 10.0 References

1. Gao, J.; Volkmann, T.; Herlach, D. M. Solidification of levitated Nd—Fe—B alloy droplets at significant bulk undercoolings J. Alloys Compounds 2003, 350, 344-350.
2. Croat, T. K.; Kelton, K. F.; Holland-Moritz, D.; Rathz, T. J.; Robinson, M. B. Containerless solidification studies of the _{—}−1/1 crystal approximant in Ti—Cr—Si—O alloys J. Mater. Res. 1999, 14, 4208-4213.
3. Bertero, G. A.; Hofmeister, W. H.; Robinson, M. B.; Bayuzick, R. J. Containerless processing and rapid solidification of niobium-silicon alloys of hypereutectic composition Metallurgical Transactions A: Physical Metallurgy and Materials Science 1991, 22A, 2723-2732.
4. Nagashio, K.; Li, M.; Kuribayashi, K. Containerless solidification and net shaping by splat quenching of undercooled Nd2Fe14B melts Materials Transactions 2003, 44, 853-860.
5. Hermann, R.; Bacher, I.; Matson, D. M.; Loser, W.; Schultz, L. Growth kinetics in levitated and quenched Nd—Fe—B alloys IEEE Trans. Magn. 2001, 37, 1100-1105.
6. Nagashio, K.; Kuribayashi, K.; Takamura, Y. Direct crystallization of Y3Fe5O12 garnet by containerless solidification processing Materials Transactions 2001, 42, 233-237.
7. Jacob, K. T.; Hajra, J. P. Electromagnetic levitation study of sulfur in liquid iron, nickel, and iron-nickel alloys Trans. Indian Inst. Met. 1986, 39, 62-69.
8. Bogan, M. J.; Agnes, G. R. MALDI-TOF-MS analysis of droplets prepared in an
electrodynamic balance: “Wall-less” sample preparation Anal. Chem. 2002, 74, 489-496.
9. Millikan, R. A. Phys. Rev. 1909, 30, 560.
10. Millikan, R. A. Phys. Rev. 1913, 2, 109-143.
11. Paul, W. Electromagnetic Traps for Charged and neutral particles Reviews of Modern Physics 1990, 62, 531-540.
12. Wuerker, R. F.; Shelton, H.; Langmuir, R. V. Electrodynamic containment of charged particles J. Appl. Phys. 1959, 30, 342.
13. Davis, E. J.; Buehler, M. F.; Ward, T. L. The double-ring electrodynamic balance for microparticle characterization Rev. Sci. Instrum. 1990, 61, 1281-1288.
14. Davis, E. J. A History of Single Aerosol Particle Levitation Aerosol Sci. Technol. 1997, 26, 212-254.
15. Davis, E. J.; Rassat, S. D.; Foss, W. Measurement of aerosol/gas reaction rates by microparticle Raman spectroscopy J. Aeosol Sci. 1992, 23,
16. Haddrell, A. E.; Agnes, G. R. Organic Cation Distributions in the Residues of Levitated Droplets with Net Charge: Validity of the Partition Theory for Droplets Produced by an Electrospray Anal. Chem. 2004, 76, 53-61.
17. Keesee, R. G.; Castleman, A. W. Thermochemical data on gas-phase ion-molecule association and clustering reactions J. Phys. Chem. Ref. Data 1986, 15, 1011-.
18. Wells, J. M.; Chrisman, P. A.; McLuckey, S. A. Formation of protein-protein complexes in vacuo J. Am. Soc. Chem. 2001, 123, 12428-12429.
19. Jang, H. M.; Hwang, N. M. Theory of the charged cluster formation in the low pressure synthesis of diamond: Part 1. Charge-induced nucleation J. Mater. Res. 1998, 13, 3527-3535.
20. Tang, I. N.; Munkelwitz, H. R. J. o. C. a. I. S.; (1984), 430-8. An investigation of solute nucleation in levitated solution droplets J. Colloid Inter. Sci. 1984, 98, 430-438.
21. Vortisch, H.; Kramer, B.; Weidinger, I.; Woste, L.; Leisner, T.; Schwell, M.; Baumgartel, H.; Ruhl, E. Homogeneous freezing nucleation rates and crystallization dynamics of single levitated sulfuric acid solution droplets Phys. Chem. Chem. Phys. 2000, 2, 1407-1413.
22. Weidinger, I.; Klein, J.; Stoeckel, P.; Baumgaertel, H.; Leisner, T. Nucleation Behavior of n-Alkane Microdroplets in an Electrodynamic Balance J. Phys. Chem. B 2003, 107, 3636-3643
23. Krieger, U. K.; Colberg, C. A.; Weers, U.; Koop, T.; Peter, T. H. Supercooling of single H2SO4/H2O aerosols to 158 K: No evidence for the occurrence of the octrahydrate Geophys. Res. Lett. 2000, 27, 2097-2100.
24. Musick, J.; Popp, J. Investigations of chemical reactions between single levitated magnesium chloride microdroplets with SO2 and NOx by means of Raman spectroscopy and elastic light scattering Phys. Chem. Chem. Phys. 1999, 1, 5497-5502.
25. Musick, J.; Kiefer, W.; Popp, J. Chemical reactions of single levitated inorganic salt particles with ammonia gas Appl. Spectrosc. 2000, 54, 1136-1141.
26. Aardahl, C. L.; Davis, E. J. Gas/aerosol chemical reactions in the NaOH—SO2-H2O system Appl. Spectrosc. 1996, 50, 71-77.
27. Anders, K.; Roth, N.; Frohn, A. New technique for investigating phase transition processes of optically levitated droplets consisting of water and sulfuric acid J. Geophys. Res., [Atmos.] 1996, 101, 19223-19229.
28. Widmann, J. F.; Aardahl, C. L.; Davis, E. J. Microparticle Raman spectroscopy Trends Anal. Chem. 1998, 17, 339-345.
29. Jacob, P.; Stockhaus, A.; Hergenroder, R.; Klockow, D. Phase transfer and freezing processes investigated on acoustically levitated aqueous droplets Fresnius' J. Anal. Chem. 2001, 371, 726-733.
30. Trunk, M.; Popp, J.; Lankers, M.; Kiefer, W. Microchemistry: Time dependence of an acid-base reaction in a single optically levitated microdroplet Chem. Phys. Lett. 1997, 264, 233-237.
31. Esen, C.; Kaiser, T. Raman investigation of photopolymerization reactions of single optically levitated microparticles Appl. Spectrosc. 1996, 50, 823-828.
32. Musick, J.; Popp, J.; Trunk, M.; Kiefer, W. Investigations of radical polymerization and copolymerization reactions in optically levitated microdroplets by simultaneous Raman spectroscopy, Mie scattering, and radiation pressure measurements Appl. Spectrosc. 1998, 52, 592-701.
33. Ward, T. L.; Zhang, S. H.; Allen, T.; Davis, E. J. Photochemical polymerization of acrylamide aerosol particles J. Colloid Interface Sci. 1987, 118, 343-355.
34. Widmann, J. F.; Davis, E. J. Photochemical initiated polymerization of single microdroplets Colloid Polym. Sci. 1996, 274, 525-531.
35. Widmann, J. F.; Aardahl, C. L.; Johnson, T. J.; Davis, E. J. Encapsulation of levitated microparticles J. Colloid Interface Sci. 1998, 199, 197-205.
36. Cederfelt, S.-I.; Martinsson, B. G.; Hansson, H.-C. On the charge limit for crystallizing particles A. Aerosol Sci. 1990, 21, S127-S130.
37. Rayleigh, L. Philos. Mag. 1882, 14, 184-186.
38. Taflin, D.C.; Ward, T. L.; Davis, E. J. Electrified droplet fission and the Rayleigh limit Langmuir 1989, 5, 376-384.
39. Smith, J. N.; Flagan, R. C.; Beauchamp, J. L. Droplet evaporation and discharge dynamics in electrospray ionization J. Phys. Chem. A 2002, 106, 9957-9967.
40. Dudek, D. R.; Wright, D. A.; Longwell, J. P.; Sarofim, A. F.; Yeheskel, J. Charge loss from heated particles levitated in an electrodynamic balance Combust. Sci. Technol. 1990, 73, 447-461.
41. Duft, D.; Achtzehn, T.; Muller, R.; Huber, B. A.; Leisner, T. Rayleigh jets from levitated microdroplets Nature 2003, 421, 128.
42. Manil, B.; Ntamack, G. E.; Lebuis, H.; Huber, B. A.; Duft, D.; Leisner, T.; Chandezon, F.; Guet, C. Charge emission and decay dynamics of highly charged clusters and micro-droplets Nucl. Instrum. Methods Phys. Res. B 2003, 205, 684-689.
43. Duft, D.; Lebuis, H.; Huber, B. A.; Guet, C.; Leisner, T. Shape oscillations and stability of charged microdroplets Phys. Rev. Lett. 2002, 89, 84503.
44. Feng, X.; Bogan, M.; Agnes, G. R. Coulomb fission event resolved progeny droplet production from isolated evaporating methanol droplets Anal. Chem. 2001, 73, 4499-4507.
45. Grimm, R. L.; Beauchamp, J. L. Evaporation and discharge dynamics of highly charged droplets of heptane, octane, and p-xylene generated by electrospray ionization Anal. Chem. 2002, 74, 6291-6297.
46. Fenn, J. B.; Mann, M.; Meng, C. K.; Wong, S. F.; Whitehouse, C. M. Electrospray ionization for mass spectrometry of large biomolecules Science 1989, 246, 64-71.
47. Kebarle, P.; Tang, L. From ions in solution to ions in the gas phase—the mechanism of electrospray mass spectrometry Anal. Chem. 1993, 65, 972A-986A.
48. Constantopoulos, T. L.; Jackson, G. S.; Enke, C. G. Challenges in achieving a fundamental model for ESI Anal. Chim. Acta 2000, 406, 37-52.
49. Gamero-Castano, M.; Fernandez de la Mora, J. Mechanisms of electrospray ionization of singly and multiply charged salt clusters Anal. Chim. Acta 2000, 406, 67-91.
50. Gamero-Castano, M.; Femadez de la Mora, J. Modulations in the abundance of salt clusters in electrosprays Anal. Chem. 2000, 72, 1426-1429.
51. Wang, G.; Cole, R. B. Charged residue versus ion evaporation for formation of alkali metal halide cluster ions in ESI (Electrospray ionization) Anal. Chim. Acta 2000, 406, 53-65.
52. Kojima, T.; Kudaka, I.; Asakawa, T.; Akiyama, R.; Kawashima, Y.; Hiraoka, K. Observation of triply charged metal ion clusters by electrospray and laser spray Rapid Commun. Mass Spectrom. 1999, 13, 2090-2097.
53. Wang, G.; Cole, R. B. Solvation Energy and Gas-Phase Stability Influences on Alkali Metal Cluster Ion Formation in Electrospray Ionization Mass Spectrometry Anal. Chem. 1998, 70, 873-881.
54. Zhou, S.; Hamurger, M. Formation of sodium cluster ions in electrospray mass spectrometry Rapid Commun. Mass Spectrom. 1996, 10, 797-800.
55. Schalley, C. A.; Weis, P. Unusually stable magic number clusters of serine with a surprising preference for homochirality. Int. J. Mass Spectrom. 2002, 221, 9-19.
56. Koch, K. J.; Aggerholm, T.; Nanita, S.C.; Cooks, R. G. Clustering of nucleobases with alkali metals studied by electrospray ionization tandem mass spectrometry: implications for mechanisms of multistrand DNA stabilization J. Mass Spectrom. 2002, 37, 676-686.
57. Vrkic, A. K.; O'Hair, R. A. J. Gas phase ion chemistry of para substituted benzene diazonium ions, their salt clusters and their related phenyl cations Int. J. Mass Spectrom. 2002, 218, 131-160.
58. Tao, W. A.; Cooks, R. G. Chiral preferences in the dissociation of homogeneous amino acid/metal ion clusters Europ. J. Mass Spectrom. 2002, 8, 107-115.
59. Carlesso, V.; Fournier, F.; Afonso, C.; Tabet, J. C. Halogen counter-ion effect on the dissociation of monosaccharide-iron complexes generated by electrospray ionization combined with an ion trap mass spectrometer Eur. J. Mass Spectrom. 2001, 7, 331-341.
60. Gaumet, J. J.; Strouse, G. Nanospray mass spectrometry technique for analysing nanomaterials from molecular precursors up to 1.5 nm in diameter cluster Materials Science & Engineering C: Biomimetic and Supramolecular Systems 2002, C19, 299-304.
61. Counterman, A. E.; Clemmer, D. E. Magic number clusters of serine in the gas phase J. Phys. Chem. B 2001, 105, 8092-8096.
62. Hernandez, H.; Hewitson, K. S.; Roach, P.; Shaw, N. M.; Baldwin, J. E.; Robinson, C. V. Observation of the Iron-Sulfur Cluster in Escherichia coli Biotin Synthase by Nanoflow Electrospray Mass Spectrometry Anal. Chem. 2001, 73, 4154-4161.
63. Hao, C.; March, R. E.; Croley, T. R.; Smith, J. C.; Rafferty, S. P. Electrospray ionization tandem mass spectrometry study of salt cluster ions. Part 1- investigations of alkali metal chloride and sodium salt cluster ions J. Mass Spectrom. 2001, 36, 79-96.
64. Hao, C.; March, R. E. Electrospray ionization tandem mass spectrometric study of salt cluster ions: part 2- Salts of polyatomic acid groups and of multivalent metals J. Mass Spectrom. 2001, 36, 509-521.
65. Lee, Y.; Jo, S.-C.; Tao, W. A.; Cooks, R. G. Metal-assisted esterification: glutaric acid-iron(II) complexes in the gas phase Rapid Commun. Mass Spectrom. 2001, 15, 484-488.
66. Lee, S.-W.; Cox, H.; Goddard, W. A., III; Beauchamp, J. L. Chemistry in Nanodroplets: Studies of Protonation Sites of Substituted Anilines in Water Clusters Using FT-ICR J. Am. Chem. Soc. 2000, 122, 9201-9205.
67. Gamero-Castano, M.; De la Mora, J. F. Kinetics of small ion evaporation from the charge and mass distribution of multiply charged clusters in electrosprays J. Mass Spectrom. 2000, 35, 790-803.
68. Zhang, D.; Wu, L.; Koch, K. J.; Cooks, R. G. Arginine clusters generated by electrospray ionization and identified by tandem mass spectrometry Eur. Mass Spectrom. 1999, 5, 353-361.
69. Zhang, D.; Cooks, R. G. Doubly charged cluster ions [(NaCl)m(Na)2]2+: magic numbers, dissociation, and structure Int. J. Mass Spectrom. 2000, 195/196, 667-684.
70. Charles, L.; Pepin, D.; Gonnet, F.; Tabet, J.-C. Effects of liquid phase composition on salt cluster formation in positive ion mode electrospray mass spectrometry: implications for clustering mechanism in electrospray J. Am. Soc. Mass Spectrom. 2001, 12, 1077-1084.
71. Gamero-Castano, M.; de la Mora, J. F. Ion-induced nucleation: Measurement of the effect of embryo's size and charge state on the critical supersaturation J. Chem. Phys. 2002, 117, 3345-3353.
72. Festag, R.; Alexandratos, S. D.; Cook, K. D.; Joy, D.C.; Annis, B.; Wunderlich, B. Single- and few-chain polystyrene particles by electrospray Macromolecules 1997, 30, 6238-6242.
73. Green, B. N.; Gotoh, T.; Suzuki, T.; Zal, F.; Lallier, F. H.; Toulmond, A.; Vinogradov, S. N. Observation of large, non-covalent globin subassemblies in the .apprx.3600 kDa hexagonal bilayer hemoglobins by electrospray ionization time-of-flight mass spectrometry J. Mol. Biol. 2001, 309, 553-560.
74. Nettleton, E. J.; Tito, P.; Sunde, M.; Bouchard, M.; Dobson, C. M.; Robinson, C. V. Characterization of the oligomeric states of insulin in self-assembly and amyloid fibril formation by mass spectrometry Biophys. J. 2000, 79, 1053-1065.
75. Lee, S.-W.; Beauchamp, J. L. Fourier transform ion cyclotron resonance study of multiply charged aggregates of small singly charged peptides formed by electrospray ionization J. Am. Soc. Mass Spectrom. 1999, 10, 347-351.
76. Thomson, B. A. Declustering and Fragmentation of Protein Ions from an Electrospray Ion Source J. Am. Soc. Mass Spectrom. 1997, 8, 1053-1058.
77. Morozov, V. N.; Morozova, T. Y.; Kallenbach, N. R. Atomic force microscopy of structures produced by electrospraying polymer solutions Int. J. Mass Spectrom. 1998, 178, 143-159.
78. Schmittberger, H.; Lierke Ernst, G.; Battelle Institute E V: USA, 1989.
79. Xie, W. J.; Cao, C. D.; Lu, Y. J.; Wei, B. Eutectic growth under acoustic levitation conditions Physical Review E: Statistical, Nonlinear, and Soft Matter Physics 2002, 66,
80. Brooks, J. S.; Reavis, J. A.; Medwood, R. A.; Stalcup, T. F.; Meisel, M. W.; Steinberg, E.; Arnowitz, L.; Stover, C. C.; Perenboom, J. A. A. J. New opportunities in science, materials, and biological systems in the low-gravity (magnetic levitation) environment (invited) J. Appl. Phys. 2000, 87, 6194-6199.
81. System and method for screening of nucleation tendency of a molecule in a levitated droplet, WO02057520, Cedergren, E.; Nilsson, et al., 2002.
82. System for performing assays on a levitated droplet, WO9944746, Nilsson, J. et al Ortho McNeil Pharm. Inc. (US), 1999.
83. Santesson, S.; Cedergren-Zeppezauer, E. S.; Johansson, T.; Laurell, T.; Nilsson, J.; Nilsson, S. Screening of nucleation conditions using levitated drops for protein crystallization Anal. Chem. 2003, 75, 1733-1740.
84. Santarsiero, B. D.; Yegian, D. T.; Lee, C. C.; Spraggon, G.; Gu, J.; Scheibe, D.; Uber, D.C.; Cornell, E. W.; Nordmeyer, R. A.; Kolbe, W. F.; Jin, J.; Jones, A. L.; Jaflevic, J. M.; Schultz, P. G.; Stevens, R. C. An approach to rapid protein crystallization using nanodroplets J. Appl. Cryst. 2002, 35, 278-281.
85. Ishikawa, Y.; Komada, S. Development of acoustic and electrostatic levitators for containerless protein crystallization Fujitsu Sci. Tech. J. 1993, 29, 330-338.
86. Chung, S. K.; Trinh, E. H. Applications of hybrid ultrasonic-electrostatic levitation to crystal growth and drop dynamics. FED (Am. Soc. Mech. Eng.) 1998, 245, 18/91-18/97.
87. Chung, S. K.; Trinh, E. H. Containerless protein crystal growth in rotating levitated drops J. Cryst. Growth 1998, 194, 384-397.
88. Chung, S. K.; Trinh, E. H. Applications of hybrid ultrasonic-electrostatic levitation to crystal growth and drop dynamics FED (American Society of Mechanical Engineers) 1998, 245, 18/91-18-97.
89. Rhim, W. K.; Chung, S. K. Containerless protein crystal growth method J. Cryst. Growth 1991, 110, 293-301.
90. Rhim, W.-K.; K. Chung, S.; Barber, D.; Man, K. F.; Gutt, G.; Rulison, A.; Spjut, R. E. An electrostatic levitator for high-temperature containerless materials processing in 1-g Rev. Sci. Instrum. 1993, 64, 2961-2970.
91. Rey, C. A.; Sisler, R.; Merkley, D. R.; Danley, T. J. Acoustic levitation for high-temperature containerless processing in space Prog. Astronaut. Aeronaut. 1990, 127 (Space Commer.: Platforms Process.), 270-285.
92. Bancel, P. A.; Cajipe, V. B.; Rodier, F. Manipulating crystals with light J. Cryst. Growth 1999, 196, 685-690.
93. Controlled nucleation of protein crystals, 2003024470 A120030206 CAN 138:145419 AN 2003:97660 CAPLUS, Myerson, A. S., 2003.
94. Zaccaro, J.; Matic, J.; Myerson, A. S.; Garetz, B. A. Nonphotochemical, laser-induced nucleation of supersaturated aqueous glycine produces unexpected g-polymorph Crystal Growth & Design 2001, 1, 5-8.
95. Park, H. K.; Grigoropoulos, C. P.; Yavas, O.; Poon, C. C.; Tam, A. C. Laser-induced nucleation and cavitation at a liquid-solid interface HTD (American Society of Mechanical Engineers) 1994, 291,
96. Binder, K. Is laser-induced nucleation due to a bulk long-wavelength instability? Journal de Plysique, Colloque 1980, C4, 75-78.
97. Wang, W.; Liao, K.; Wang, B.; Xiao, J. In Proceedings of SPIE-The International Society for Optical Engineering (1999), 1999; Vol. 3862 (Industrial Lasers (IL '99)), pp 479-483.
98. Gadomski, A.; Siodmiak, J. A novel model of protein crystal growth: Kinetic limits, length scales and the role of the double layer Croatia Chemica Acta 2003, 76, 129-136.
99. Sear, R. P. Protein crystals and charged surfaces: Interactions and heterogeneous nucleation Phys. Rev. E 2003, 67, 061907/1-011907/7.
100. Sear, R. P. Distribution of the second virial coefficients of globular proteins Europhys. Let. 2002, 60, 938-944.
101. Sear, R. P.; Warren, P. B. On the electrical double layer contribution to the interfacial tension of protein crystals J. Chem. Phys. 2002, 117, 8074-8079.
102. Avassoli, Z.; Sear, R. P. Homogeneous nucleation near a second phase transition and Ostwald's step rule J. Chem. Phys. 2002, 116, 5066-5072.
103. Bechtold, I. H.; De Santo, M. P.; Bonvent, J. J.; Oliveira, E. A.; Barberi, R.; Rasing, T. Rubbing-induced charge domains observed by electrostatic force microscopy: effect on liquid crystal alignment Liquid Crystals 2003, 30, 591-598.
104. Schmitz, K. S. Surface Charge Induced Reentrant Crystalline-Liquid Transition in Colloidal Systems: The Role of the Microion Disposition Langmuir 2001, 17, 8028-8039.
105. Thomas, N. E.; Coakley, W. T. Localized contact formation by erythrocyte membranes: electrostatic effects Biophys. J. 1995, 69, 1387-1401.
106. Coakley, W. T.; Gallez, D.; Thomas, N. E.; Baker, A. J. An interfacial instability approach to erythrocyte adhesion by macromolecules Colloids Surf., B 1994, 2, 281-290.
107. Klotz, S. A. The contribution of electrostatic forces to the process of adherence of Candida albicans yeast cells to substrates FEMS Microbiol. Lett. 1994, 120, 257-262.
108. Bunt, C. R.; Jones, D. S.; Tucker, I. G. The effects of pH, ionic strength and organic phase on the bacterial adhesion to hydrocarbons (BATH) test Int. J. Pharm. 1993, 99, 93-98.
109. Vernhet, A.; Leveau, J. Y.; Cerf, O.; Bellon-Fontaine, M. N. Role of electrostatic interactions in Saccharomyces cerevisiae adhesion to the inner surface of champagne bottles. Biofouling 1992, 5, 323-334.
110. Chang, Y. I. The effect of cationic electrolytes on the electrostatic behavior of cellular surfaces with ionizable groups J. Theor. Biol. 1989, 139, 561-571.
111. Bengtsson, G.; Lindqvist, R.; Piwoni, M. D. Sorption of trace organics to colloidal clays, polymers, and bacteria Soil Sci. Soc. Am. J. 1993, 57, 12961-12970.
112. Clegg, S.; Forster, C. F.; Crabtree, R. W. An examination into the attachment of bio-organic material to the mineral particles in sewer sediments Environ. Tech. 1993, 14, 463-470.
113. Hanczyc, M. M.; Fujikawa, S. M.; Szotstal, J. W. Experimental models of primitive cellular compartments: Encapsulation, growth, and division Science 2003, 302, 618-622.
114. Ertem, G.; Ferris, J. P. Template-directed synthesis using the heterogeneous templates produced by montmorillonite catalysis: A possible bridge between the prebiotic and RNA worlds J Am. Chem. Soc. 1997, 119, 7197-7201.
115. Ferris, J. P.; Hill, A. R.; Liu, R.; Orgel, L. E. Synthesis of long prebiotic oligomers on mineral surfaces Nature 1996, 381, 59-61.
116. Prakash, J.; Ferris, J. P.; Pitsch, S. Homochiral selection in the montmorillonite-catalyzed and uncatalyzed prebiotic synthesis of RNA Chem. Comm. 2000, 24, 2497-2498.
117. Sowerby, S. J.; Edelwirth, M.; Heckl, W. M. Self-assembly at the prebiotic solid-liquid interface: Structures of self-assembled monolayers of adenine and guanine bases formed on inorganic surfaces J. Phys. Chem. B 1998, 102, 5914-5922.
118. Huber, C.; Wachtershauser, G. Peptides by activation of amino acids with CO on (Ni,Fe)S surfaces: Implications for the origin of life Science 1998, 281, 670-672.
119. Huber, C.; Wachtershauser, G. Activated acetic acid by carbon fixation on (Fe,Ni)S under primordial conditions Science 1997, 276, 245-247.
120. Cody, G. D.; Boctor, N. Z.; Filley, T. R.; Hazen, R. M.; Scott, J. H.; Sharma, A.; Yoder, H. S. Primordial carbonylated iron-sulfur compounds and the synthesis of pyruvate Science 2000, 289, 1337-1340.
121. Smith, J. V.; Arnold, F. P.; Parsons, I.; Lee, M. R. Biochemical evolution III: Polymerization on organophilic silica-rich surfaces, crystal-chemical modeling, formation of first cells, and geological clues Proc. Natl. Acad. Sci. USA 1999, 96, 3479-3485.
122. Smith, J. V. Biochemical evolution. I. Polymerization on internal, organophilic silica surfaces of dealuminated zeolites and feldspars Proc. Natl. Acad. Sci. USA 1998, 95, 3370-3375.
123. Williams, R. J. P. The fundamental nature of life as a chemical system: the part played by inorganic elements J. Inorg. Biochem. 2002, 88, 241-250.
124. Bogan, M. J.; Agnes, G. R. Wall-less sample preparation of micrometer-sized sample spots for femtomole detection limits of proteins from liquid based UV-MALDI matrices 2005, 16 J. Am. Soc. Mass Spectrom., 254-262.
125 Agnes, G. R.; Horlick, G. Appl. Spectrosc. 1994, 48, 649-654.
126. Xu, Y.; Bruening, M. L.; Watson, J. T. Non-specific, on-probe cleanup methods for MALDI-MS samples Mass Spectrom. Rev. 2003, 22, 429-440.
127. Luxembourg, S. L.; McDonnell, L. A.; Duursma, M. C.; Guo, X.; Heeren, R. M. A. Effect of local matrix crystal variations in matrix-assisted ionization techniques for mass spectrometry Anal. Chem. 2003, 75, 2333-2341.
128. McDonnell, L. A.; Mize, T. H.; Luxembourg, S. L.; Koster, S.; Eijkel, G. B.; Verpoorte, E.; de Rooij, N. F.; Heeren, R. M. A. Using matrix peaks to map topography: Increased mass resolution and enhanced sensitivity in chemical imaging Anal. Chem. 2003, 75, 4373-4381.
129. Michelle L. Shulman, Robert J. Charlson, E. James Davis, The effects of atmospheric organics on aqueous droplet formation, J. Aerosol Sci., 1997, 28, 737-752.
130. Ray, A. K.; Johnson, R. D.; Souyri, A. Dynamic behaviour of single glycerol droplets in humid air streams, Langmuir, 1989, 5, 133-140.
131. Duft, D.; Lebuis, H.; Huber, B. A.; Guet, C.; Leisner, T. Phys. Rev. Lett. 2002, 89, 84503.
132. Ivarone, A. T.; Udekwu, O. A.; Willimams E. R., Buffer loading for counteracting metal salt-induced signal suppression in electrospray ionization, Anal. Chem., 2004, 76, 3944-3950.
133. Weidinger, I.; Klein, J.; Stockel, P.; Baumgartel, H.; Leisner, T. Nucleation behaviour of n-alkane microdroplets in an electrodynamic balance, J. Phys. Chem. B 2003, 107, 3636-3643.
134. Zhu, J.; Zheng, F.; Laucks, M. L.; Davis, E. J. Mass Transfer from an oscillating microsphere, J. Colloid Interface Sci., 2002, 249, 351-358.
135. R. E. March, Ion Trap Mass Spectrometry, Int. J. Mass Spectrom. Ion Processes, 1992, 118/119, 71-135.
136. Cohen, M.D.; Flagan, R. C.; Seinfeld, J. C. J. Phys. Chem. 1987, 91, 4583-4590.
137. Krämer, B.; Hübner, O.; Vortisch, H.; Wöste, L.; Leisner, T.; Schwell, M.; Rühl, E.; Baumgärtel, H. J. Chem. Phys. 1999, 111, 6521-6527.
138. Weidinger, I.; Klein, J.; Stockel, P.; Baumgartel, H.; Leisner, T. J. Phys. Chem. B 2003, 107, 3636-3643.
139. Duft, D.; Leisner, T. Atmos. Chem. Phys. Discuss. 2004, 4, 3077-3088.
140. Djikaev, Y. S.; Tabazadeh, A.; Reiss, H. J. Chem. Phys. 2003, 118, 6572-6581.
141. Tabazadeh, A.; Djikaev, Y. S.; Reiss, H. Proc. Natl. Acad. Sci. 2002, 99, 15873-15878.
142. Myland, J. C.; Oldham, K. B. J. Electroanal. Chem. 2002, 522, 115-123.
143. Wilson, C. T. R. Phil. Trans. Roy. Soc. Lon. Ser. A 1899, 192, 403-453.
144. Rabeony, H.; Mirabel, P. J. Phys. Chem. 1987, 91, 1815-1818.
145. Castleman, A. W.; Holland, P. M.; Keesee, R. G. J. Chem. Phys. 1978, 68, 1760-1767.
146 Castleman, A. W.; Tang, I. N. J. Chem. Phys. 1972, 57, 3629-2638.
147 Banic, C. M.; Iribarne, J. V. J. Chem. Phys. 1985, 83, 6432-6448.
148 Jang, H. M.; Hwang, N. M. J. Mater. Res. 1998, 13, 3527-3535.
149 Jang, H. M.; Hwang, N. M. J. Mater. Res. 1998, 13, 3536-3549.
150 Blanchard, D.C. J. Meteor. 1958, 15, 383-396.
151 Reiter, R. J. Geophys. Res. 1994, 99, 10807-10812.
152 Smith, J. N.; Flagan, R. C.; Beauchamp, J. L. J. Phys. Chem. A 2002, 106, 9957-9967.
153 Kebarle, P.; Tang, L. Anal. Chem. 1993, 65, 972A-986A.
154 Ivarone, A. T.; Udekwu, O. A.; Willaims, E. R. Anal Chem 2004, 76, 3944-3950
155 Julian, R. R.; Hodyss, R.; Kinnear, B.; Jarrold, M. F.; Beauchamp, J. L. J. Phys. Chem. B. 2002, 106, 1219-1228.
156 Takats, Z.; Cooks, R. G. Chem. Comm. 2004, 444-445
157 Hanton, S. A.; Hyder, I. Z.; Stets, J. R.; Owens, K. G.; Blair, W. R.; Guttman, C. M.; Giuseppetti, A. A. J. Am. Soc. Mass Spectrom. 2004, 15, 168-179.
158 Bogan, M. J.; Agnes, G. R. J. Am. Soc. Mass Spectrom. 2004, 15, 486-495

1. A method of controllably inducing nucleation of a first solute dissolved in a solution comprising
(a) providing a primary vessel for containing said solution;
(b) applying an induction potential to said primary vessel such that said solution acquires a net charge; and
(c) causing ion-induced nucleation of at least some of said first solute in a condensed phase.

2. The method as defined in claim 1, wherein said step of causing ion-induced nucleation comprises maintaining the surface charge density of said primary vessel above a threshold value.

3. The method as defined in claim 1, wherein causing ion-induced nucleation comprises adjusting the surface charge density of said primary vessel.

4. The method as defined in claim 1, wherein ions in said vessel in excess of any counterions induce heterogeneous nucleation of said solute.

5. The method as defined in claim 1, wherein said primary vessel is wall-less.

6. The method as defined in claim 1, wherein said primary vessel is a droplet.

7. The method as define in claim 6, wherein said droplet is levitated after application of said induction potential.

8. The method as defined in claim 7, wherein said droplet is levitated in a levitation device selected from the group consisting of an electrodynamic balance, an acoustic balance, an electrostatic balance, a magnetic balance, a radiation pressure device or any combination thereof.

9. The method as defined in claim 1, wherein said ion-induced nucleation causes formation of one or more nuclei and wherein said method further comprises delivering said nuclei to a target location.

10. The method as defined in claim 9, wherein said target location is a substrate.

11. The method as defined in claim 10, wherein said primary vessel is a droplet levitated in a levitation device and wherein said target location is located at a position remote from said levitation device.

12. The method as defined in claim 10, wherein at least a portion of said solution comprising said nuclei is deposited on said substrate.

13. The method as defined in claim 9, wherein at least some of said nuclei are delivered from said primary vessel to a secondary vessel.

14. The method as defined in claim 13, wherein said at least some of said nuclei seed crystal growth in said secondary vessel.

15. The method as defined in claim 1, wherein said solution initially comprises at least one volatile solvent, and wherein said solvent evaporates from said vessel causing an increase in concentration of said first solute.

16. The method as defined in claim 1, herein volatile solvents in said solution are allowed to evaporate to yield a residue comprising said one or more nuclei.

17. The method as defined in claim 4, wherein said ions causing said heterogeneous nucleation are located in an outer layer of said vessel.

18. The method as defined in claim 6, wherein said solution comprises a surface tension modifier to inhibit Coulomb explosion of said droplet.

19. The method as defined in claim 9, wherein at least some of said nuclei are used to promote crystallization of said first solute.

20. The method as defined in claim 1, wherein said first solute is a solid.

21. The method as defined in claim 1, wherein said first solute is selected from the group consisting of inorganic compounds and organic compounds.

22. The method as defined in claim 1, wherein said first solute is a melt.

23. The method as defined in claim 22, wherein said first solute is a biomolecule.

24. The method as defined in claim 23, wherein said biomolecule is a protein.

25. The method as defined in claim 22, wherein said first solute is an organic acid.

26. The method as defined in claim 22, wherein said organic compound is selected from the group consisting of CHCA and THAP.

27. The method as defined in claim 1, wherein a second solute is dissolved in said solution in addition to said first solute.

28. The method as defined in claim 26, wherein said first solute and said second solute are selected from the group consisting of organic compounds and inorganic compounds.

29. The method as defined in claim 27, comprising selectively precipitating at least one said first and second solutes.

30. The method as defined in claim 29, comprising separating one said first and second solutes from the other of said first and second solutes by selectively causing crystallization of one of said solutes.

31. The method as defined in claim 28, wherein said first and second solutes are stereoisomers.

32. The method as defined in claim 28, wherein said first and second solutes are enantiomers.

33. The method as defined in claim 28, wherein said method results in co-crystallization of said first and second solutes.

34. The method as defined in claim 33, wherein said second solute is a MALDI matrix.

35. The method as defined in claim 1, wherein said method is adapted for selectively separating polymorphic forms of said first solute.

36. The method as defined in claim 1, wherein said primary vessel comprises a portion of a conduit holding said solution.

37. The method as defined in claim 36, wherein said conduit is a capillary.

38. The method as defined in claim 27, wherein said method causes differential precipitation of said first and second solutes.

39. The method as defined in claim 1, further comprising adding a solid to said solution to further induce nucleation of said first solute.

40. The method as defined in claim 1, optimizing the ionic make-up of said solution prior to applying said induction potential.

41. A precipate or co-precipitate produced by the method of claim 1.

42. A method of controllably inducing precipitation of selected solutes dissolved in a solution comprising
(a) providing a primary vessel for containing said solution;
(b) applying an induction potential to said primary vessel such that said solution acquires a net charge; and
(c) selectively causing ion-induced precipitation of at least one of said solutes in a condensed phase.

43. The method as defined in claim 42, wherein said step of selectively causing ion-induced precipitation comprises adjusting the mass-to-charge ratio of said primary vessel.

44. The method as defined in claim 42, wherein said step of selectively causing ion-induced precipitation comprises increasing the surface charge density of said vessel.

45. The method as defined in claim 44, wherein said primary vessel is a droplet and said surface charge density is increased at an outer air-droplet interface thereof.

46. A method of controllably inducing crystallization of at least one solute dissolved in a solution, said method comprising
(a) providing a primary vessel comprising said solution;
(b) controllably imparting a net charge on said solution in a condensed phase to selectively cause ion-induced nucleation of said at least one solute; and
(c) depositing crystals derived from said nucleation on a substrate.

47. The method as defined in claim 46, wherein said crystals are deposited on said substrate at a predetermined target location.

48. The method as defined in claim 46, further comprising identifying the structure of said crystals on said substrate.

49. The method as defined in claim 46, further comprising producing said solute on said substrate in a purified solid form.

50. The method as defined in claim 46, wherein said depositing and said identifying are automated.

51. The method as defined in claim 46, wherein said solution comprises a mixture of solutes dissolved in said solution and wherein steps (a)- (c) are performed in respect of each of a plurality of said solutes to thereby selectively separate and characterize said solutes.

52. The method as defined in claim 51, wherein said solutes comprise biomolecules.

53. The method as defined in claim 51, wherein said solutes comprise stereoisomers.

54. The method as defined in claim 51, wherein said solutes comprise a mixture of organic and inorganic compounds.

55. The method as defined in claim 51, wherein said solutes are selected from the group consisting of metals, alloys and melts

56. The method as defined in claim 51, wherein at least one of said solutes is a MALDI matrix.

57. The method as defined in claim 51, wherein as least one of said solutes is a polymorph.1.
